(19) United States
(12) Patent Application Publication
(10) Pub. No.: US 2019/0269716 A1
(43) Pub. Date: Sep. 5, 2019

(54) COMPOSITIONS AND METHODS FOR PROGNOSING AND TREATING COLORECTAL CANCER

(71) Applicants: VENTANA MEDICAL SYSTEMS, INC., Tucson, AZ (US); ROCHE SEQUENCING SOLUTIONS INC., Pleasanton, CA (US); Charité - Universitätsmedizin Berlin Gliedkörperschaft der Freien Universität Berlin Und der Humbold, Berlin (DE)

(72) Inventors: Pia Herrmann, Berlin (DE); Katharina Ilm, Berlin (DE); Katherine F. Leith, Tucson, AZ (US); Andrea Muranyi, Tucson, AZ (US); Ulrich-Peter Rohr, Pleasanton, CA (US); Kandavel Shanmugam, Tucson, AZ (US); Shalini Singh, Tucson, AZ (US); Ulrike Stein, Berlin (DE)

(21) Appl. No.: 16/412,057

(22) Filed: May 14, 2019

Related U.S. Application Data
(63) Continuation of application No. PCT/EP2017/079084, filed on Nov. 13, 2017.
(60) Provisional application No. 62/422,573, filed on Nov. 15, 2016.

(51) Int. Cl. A61K 31/7072; A61P 35/04; C12Q 1/686; G01N 1/30; A61K 31/52; A61K 31/513; C12Q 1/6886; G01N 21/88

(52) U.S. Cl. CPC A61K 31/7072; A61P 35/04; C12Q 1/686; G01N 1/30; A61K 31/52; A61K 31/513; C12Q 1/6818; G01N 21/8806; C12Q 2561/113; C12Q 2600/118; C12Q 2600/16; C12Q 2563/179; C12Q 2563/107; C12Q 1/6886

(57) ABSTRACT

A combination of mismatch repair (MMR) and Metastasis Associated in Colon Cancer 1 (MACC1) gene expression status of the patient serve as a basis for risk stratification of early stage colon cancer patients. Patients with defective MMR (dMMR) status have improved survival and do not benefit from 5-fluorouracil (5-FU) therapies. In contrast, patients with a proficient MMR (pMMR) status have a higher risk of recurrence and worse survival. The pMMR patients are then further stratified on the basis of MACC1 gene expression. Patients with a pMMR status and a low MACC1 expression have a favorable prognosis similar to patients having a dMMR status, whereas patients having a pMMR status and high MACC1 expression have a less favorable prognosis.

### CROSS-REFERENCE TO RELATED APPLICATIONS

This application is a by-pass continuation of International Patent Application No. PCT/EP2017/079084, filed 13 Nov. 2017, which claims priority to U.S. Provisional Patent Application No. 62/422,573, filed 15 Nov. 2016. The disclosure of the priority applications are incorporated in their entirety herein by reference.

### REFERENCE TO SEQUENCE LISTING SUBMITTED AS A COMPLIANT ASCII TEXT FILE (.txt)

Pursuant to the EFS-Web legal framework and 37 CFR §§ 1.821-825 (see MPEP § 2442.03(a)), a Sequence Listing in the form of an ASCII-compliant text file (entitled “Sequence_Listing_3000022-008000_ST25.txt” created on 6 May 2019, and 67,719 bytes in size) is submitted concurrently with the instant application, and the entire contents of the Sequence Listing are incorporated herein by reference.

### BACKGROUND OF THE INVENTION

Early-stage colon cancers (stage I-III), account for >70% of all patients with this cancer. (Siegel et al 2014). Even if we only focus on patients with stage II colon cancer, who represent 25% of this patient group (Edge et al. 2010), it is still demanding for physicians to discuss the risk/benefit profile of adjuvant chemotherapy with their patients, particularly if they are elderly. When deciding on a treatment course the oncologist must use their clinical expertise to look holistically at the individual clinical situation of their patient, including age, comorbidities, performance status, and most importantly the patient's wishes, while also bearing in mind any relevant clinical guidelines. The US National Comprehensive Cancer Network (NCCN) guidelines (National Comprehensive Cancer Network, 2016) suggest multiple treatment options for patients with stage II disease, ranging from observation to a variety of chemotherapy modalities. These options are based on the observation derived from meta-analyses and large randomized trials, such as the Quasar Study (Figueredo et al. 2004; Mamounas et al. 1999; Quasar Collaborative Group et al. 2007), that there is no—or only marginal—gain with fluoropyrimidine-based adjuvant chemotherapy if all patients with colon cancer are treated at stage II, and thus it cannot be recommended as a standard of care. Unfortunately, approximately 15-20% of patients with stage II disease experience a relapse after curative surgery (Shi et al. 2013).

Diagnostic assays may provide meaningful additional information regarding which patients have a high likelihood of relapse and hence would gain potential benefit from additional adjuvant chemotherapy (Shi et al. 2013). Despite the identification of high-risk clinicopathological features, such as poor histology, bowel perforation, inadequate lymph node sampling, lymphovascular or perineural invasion, and invasion of adjacent organs or structures (T4 extension), there is no evidence for an adequate predictor of either relapse or chemotherapeutic benefit in these high-risk patients. Some gene scores and classifiers have been developed (Kopetz et al. 2015; Sveen et al. 2013) and have been validated in patient cohorts. Despite the modest association with prognosis, these tests come at high costs and have not been recommended for routine use so far.

Therefore, there is an unmet need for molecular markers that can distinguish patients with colon and colorectal cancers who are at high-risk for relapse.

### SUMMARY OF THE INVENTION

Provided herein are kits, assays, and methods for evaluating MMR status and for detecting expression of MACC1 gene products, and methods of treatment for individuals with cancer, e.g., colon and colorectal cancers.

In an embodiment, a method of prognosing a subject having a mismatch repair-proficient (pMMR) colorectal cancer is provided, said method comprising detecting an expression level of a Metastasis Associated in Colon Cancer 1 (MACC1) gene product in a sample of the colorectal cancer and comparing the expression level to a reference expression level, wherein the subject has a unfavorable prognosis if the expression level of the MACC1 gene product exceeds the reference expression level, and wherein the subject has a favorable prognosis if the expression level of the MACC1 gene product falls below the reference expression level.

In an embodiment, a method of identifying a subject having a mismatch repair-proficient (pMMR) colorectal cancer likely to benefit from a chemotherapy is provided, said method comprising detecting an expression level of a Metastasis Associated in Colon Cancer 1 (MACC1) gene product in a sample of the colorectal cancer and comparing the expression level to a reference expression level, wherein the subject is likely to benefit from chemotherapy if the expression level of the MACC1 gene product exceeds the reference expression level, and wherein the subject wherein the subject is less likely to benefit from chemotherapy based on a treatment independent favorable prognosis if the expression level of the MACC1 gene product falls below the reference expression level. In embodiments, the chemotherapy is a fluoropyrimidine-based chemotherapy, such as capecitabine, floxuridine, or 5-fluorouracil (5-FU) with or without leucovorin.

In an embodiment, a method of treating a subject suffering from a mismatch repair-proficient (pMMR) colorectal cancer is provided, said method comprising:
detecting an expression level of a Metastasis Associated in Colon Cancer 1 (MACC1) gene product in a sample of the colorectal cancer;
comparing the expression level of the MACC1 gene product to a reference expression level,
administering a chemotherapy to the subject if the expression level of the MACC1 gene product exceeds the reference expression level,
administering a treatment course that does not include the chemotherapy or no treatment if the expression level of the MACC1 gene product falls below the reference expression level.

In an embodiment, a chemotherapeutic for use in a method of treating a subject suffering from a mismatch repair-proficient (pMMR) colorectal cancer is provided, comprising:
administering the chemotherapeutic to the subject detected to have an expression level of a Metastasis Associated in Colon Cancer 1 (MACC1) gene product in a sample of the colorectal cancer that exceeds a reference expression level of the MACC1 gene product.

In an embodiment, a chemotherapeutic for use in a method of treating a subject suffering from a mismatch repair-proficient (pMMR) colorectal cancer is provided, comprising:
detecting an expression level of a Metastasis Associated in Colon Cancer 1 (MACC1) gene product in a sample of the colorectal cancer;
comparing the expression level of the MACC1 gene product to a reference expression level,
administering the chemotherapeutic to the subject if the expression level of the MACC1 gene product exceeds the reference expression level.

In an embodiment, the use of a chemotherapeutic for the manufacture of a composition or pharmaceutical composition for treating a subject suffering from a mismatch repair-proficient (pMMR) colorectal cancer is provided, whereby the subject has been detected to have an expression level of a Metastasis Associated in Colon Cancer 1 (MACC1) gene product in a sample of the colorectal cancer that exceeds a reference expression level of the MACC1 gene product

In embodiments, the chemotherapy is a fluoropyrimidine-based chemotherapy or the chemotherapeutic is fluoropyrimidine, such as capecitabine, floxuridine, or fluorouracil (5-FU).

In an embodiment, a non-chemotherapeutic for use in a method of treating a subject suffering from a mismatch repair-proficient (pMMR) colorectal cancer is provided, comprising:
administering the non-chemotherapeutic to the subject detected to have an expression level of a Metastasis Associated in Colon Cancer 1 (MACC1) gene product in a sample of the colorectal cancer that falls below a reference expression level of the MACC1 gene product.

In an embodiment, a non-chemotherapeutic for use in a method of treating a subject suffering from a mismatch repair-proficient (pMMR) colorectal cancer is provided, comprising:
detecting an expression level of a Metastasis Associated in Colon Cancer 1 (MACC1) gene product in a sample of the colorectal cancer;
comparing the expression level of the MACC1 gene product to a reference expression level,
administering the non-chemotherapeutic to the subject if the expression level of the MACC1 gene product falls below the reference expression level.

In an embodiment, the use of a non-chemotherapeutic for the manufacture of a composition or pharmaceutical composition for treating a subject suffering from a mismatch repair-proficient (pMMR) colorectal cancer is provided, whereby the subject has been detected to have an expression level of a Metastasis Associated in Colon Cancer 1 (MACC1) gene product in a sample of the colorectal cancer that falls below a reference expression level of the MACC1 gene product.

In an embodiment, a kit is provided for detection of a mismatch repair-proficient (pMMR) colorectal cancer likely to progress and/or respond to chemotherapy, said kit comprising:
a set of MMR-associated biomarker-specific agents, and a set of detection reagents suitable for detecting binding of the MMR-associated biomarker-specific agents to a sample of the colorectal cancer; and
one or more MACC1 gene product-biomarker-specific agents, and a set of detection reagents for detecting binding of the MACC1 gene product-biomarker-specific agent to a sample of the colorectal cancer.

In a specific embodiment, a method is provided comprising:
(a) obtaining a sample of a colorectal cancer from a subject;
(b) contacting the sample with:
   (b1) an antibody specific for MLH1;
   (b2) an antibody specific for MSH2;
   (b3) an antibody specific for MSH6;
   (b4) an antibody specific for PMS2;
   (b5) an antibody specific for MACC1; and
   (b6) detection reagents sufficient for visualizing binding of each of (b1)-(b6) via brightfield or darkfield microscopy
(c) detecting via brightfield or darkfield microscopy the presence or absence of binding of each of (b1)-(b4) to the sample, and the quantity of binding of (b5) to the sample, wherein:
   (c1) the subject has a favorable prognosis if:
      (c1a) binding of at least one of (b1)-(b4) is absent, or
      (c1b) binding of each of (b1)-(b4) is present and the number of viable tumor cells having (b5) bound thereto is below a threshold level; and
   (c2) the subject has a unfavorable prognosis if binding of each of (b1)-(b4) is present and the number of viable tumor cells having (b5) bound thereto is above a threshold level.

In another specific embodiment, a method is provided comprising:
(a) obtaining a sample of a colorectal cancer from a subject;
(b) generate a composition comprising cDNA generated from mRNA of the sample by contacting a first portion of the sample with reagents sufficient for performing reverse transcription;
(c) contacting the composition comprising cDNA with:
   (c1) a primer pair specific for MLH1 cDNA;
   (c2) a primer pair specific for MSH2 cDNA;
   (c3) a primer pair specific for MSH6 cDNA;
   (c4) a primer pair specific for PMS2 cDNA;
   (c5) a primer pair specific for MACC1 cDNA; and
   (c6) reagents sufficient for amplifying the MLH1 cDNA, MSH2 cDNA, MSH6 cDNA, PMS2 cDNA, and MACC1 cDNA if present;
(d) detecting the presence or absence of MLH1 cDNA, MSH2 cDNA, MSH6 cDNA, and PMS2 cDNA, and the quantity of MACC1 cDNA, wherein:
   (d1) the subject has a favorable prognosis if:
      (d1a) at least one of MLH1 cDNA, MSH2 cDNA, MSH6 cDNA, PMS2 cDNA is absent, or
      (d1b) each of MLH1 cDNA, MSH2 cDNA, MSH6 cDNA, PMS2 cDNA is present and the quantity of MACC1 cDNA is below a threshold level; and
   (d2) the subject has a unfavorable prognosis if each of MLH1 cDNA, MSH2 cDNA, MSH6 cDNA, PMS2 cDNA is present and the quantity of MACC1 cDNA is above a threshold level.

In a specific embodiment, a method is provided comprising:
(a) obtaining a sample of a colorectal cancer from a subject;
(b) generating a composition comprising cDNA generated from mRNA of the sample by contacting a first portion of the sample with reagents sufficient for performing reverse transcription;
(c) contacting the composition comprising cDNA with:
   (c1) a primer pair specific for MLH1 cDNA;
   (c2) a primer pair specific for MSH2 cDNA;
   (c3) a primer pair specific for MSH6 cDNA;
   (c4) a primer pair specific for PMS2 cDNA; and
   (c5) reagents sufficient for amplifying the MLH1 cDNA, MSH2 cDNA, MSH6 cDNA, and PMS2 cDNA if present;
(d) contacting a second portion of the sample with:
   (e1) an antibody specific for MACC1; and
   (e2) detection reagents sufficient for visualizing binding of the antibody specific for MACC1 via brightfield or darkfield microscopy;
(f) detecting:
   (f1) the presence or absence of MLH1 cDNA, MSH2 cDNA, MSH6 cDNA, and PMS2 cDNA; and
   (f2) the quantity of binding of the antibody specific for MACC1 to the second portion of the sample via brightfield or darkfield microscopy; wherein:
      (f1) the subject has a favorable prognosis if:
         (f1a) at least one of MLH1 cDNA, MSH2 cDNA, MSH6 cDNA, PMS2 cDNA is absent, or
         (f1b) each of MLH1 cDNA, MSH2 cDNA, MSH6 cDNA, PMS2 cDNA is present and the quantity of binding of MACC1 to the second portion of the sample is below a threshold level; and
   (f2) the subject has a unfavorable prognosis if each of MLH1 cDNA, MSH2 cDNA, MSH6 cDNA, PMS2 cDNA is present and the quantity of binding of MACC1 antibody to the second portion of the sample is above a threshold level.

In a specific embodiment, a method is provided comprising:
(a) obtaining a sample of a colorectal cancer from a subject;
(b) contacting a first portion of the sample with:
   (b1) an antibody specific for MLH1;
   (b2) an antibody specific for MSH2;
   (b3) an antibody specific for MSH6;
   (b4) an antibody specific for PMS2; and
   (b5) detection reagents sufficient for visualizing binding of each of (b1)-(b5) via brightfield or darkfield microscopy
(c) generating a composition comprising cDNA generated from mRNA of the sample by contacting a second portion of the sample with reagents sufficient for performing reverse transcription;
(d) contacting the composition comprising cDNA with:
   (d1) a primer pair specific for MACC1 cDNA; and
   (d2) reagents sufficient for amplifying the MLH1 cDNA, MSH2 cDNA, MSH6 cDNA, and PMS2 cDNA if present;
(e) detecting:
   (e1) the presence or absence of binding of each of (b1)-(b4) to the first portion of the sample via brightfield or darkfield microscopy, and
   (e2) the quantity of MACC1 cDNA in the second portion of the sample, wherein:
      (e1) the subject has a favorable prognosis if:
         (e1a) binding of at least one of (b1)-(b4) is absent, or
         (e1b) binding of each of (b1)-(b4) is present and the quantity of MACC1 cDNA is below a threshold level; and
      (e2) the subject has a unfavorable prognosis if binding of each of (b1)-(b4) is present and the quantity of MACC1 cDNA is above a threshold level.

The foregoing methods may further comprise treating the subject, wherein:
the treatment does not comprise a chemotherapy if:
   at least one of a MLH1, MSH2, MSH6, PMS2 gene product is absent, or
   each of MLH1, MSH2, MSH6, PMS2 is present and the quantity of MACC1 is below a threshold level; and
the treatment comprises a chemotherapy if each of MLH1, MSH2, MSH6, PMS2 is present and the quantity of MACC1 cDNA is above a threshold level.

In an embodiment, a chemotherapeutic for use in a method for treating a subject suffering from a mismatch repair-proficient (pMMR) colorectal cancer is provided, comprising:

each of the steps (a), (b), (c), (d), (e), and (f), as present, of the forgoing specific embodiments of the methods; and

administering the chemotherapeutic to the subject detected that each of MLH1, MSH2, MSH6, PMS2 is present in a sample of the colorectal cancer and the quantity of MACC1 cDNA in a sample of the colorectal cancer is above a threshold level.

In an embodiment, a chemotherapeutic for use in a method for treating a subject suffering from a mismatch repair-proficient (pMMR) colorectal cancer is provided, comprising:

administering the chemotherapeutic to the subject detected to have each of MLH1, MSH2, MSH6, PMS2 present in a sample of the colorectal cancer and the quantity of MACC1 cDNA in a sample of the colorectal cancer is above a threshold level.

In an embodiment, the use of a chemotherapeutic for the manufacture of a composition or pharmaceutical composition for treating a subject suffering from a mismatch repair-proficient (pMMR) colorectal cancer is provided, whereby the subject has been detected to have each of MLH1, MSH2, MSH6, PMS2 present in a sample of the colorectal cancer and the quantity of MACC1 cDNA in a sample of the colorectal cancer is above a threshold level.

In an embodiment, a non-chemotherapeutic for use in a method for treating a subject suffering from a mismatch repair-proficient (pMMR) colorectal cancer is provided, comprising:

each of the steps (a), (b), (c), (d), (e), and (f), as present, of the forgoing specific embodiments of the methods; and

administering the non-chemotherapeutic to the subject detected to have at least one of a MLH1, MSH2, MSH6, PMS2 gene product absent in a sample of the colorectal cancer, or each of MLH1, MSH2, MSH6, PMS2 is present in a sample of the colorectal cancer and the quantity of MACC1 in a sample of the colorectal cancer is below a threshold level.

In an embodiment, a non-chemotherapeutic for use in a method for treating a subject suffering from a mismatch repair-proficient (pMMR) colorectal cancer is provided, comprising:

administering the non-chemotherapeutic to the subject detected to have at least one of a MLH1, MSH2, MSH6, PMS2 gene product absent in a sample of the colorectal cancer, or each of MLH1, MSH2, MSH6, PMS2 is present in a sample of the colorectal cancer and the quantity of MACC1 in a sample of the colorectal cancer is below a threshold level.

In an embodiment, the use of a non-chemotherapeutic for the manufacture of a composition or pharmaceutical composition for treating a subject suffering from a mismatch repair-proficient (pMMR) colorectal cancer is provided, whereby the subject has been detected to have at least one of a MLH1, MSH2, MSH6, PMS2 gene product absent in a sample of the colorectal cancer, or each of MLH1, MSH2, MSH6, PMS2 is present in a sample of the colorectal cancer and the quantity of MACC1 in a sample of the colorectal cancer is below a threshold level.

In any of the foregoing methods or chemotherapeutics or non-chemotherapeutics for use in a method for treating a subject suffering from a mismatch repair-proficient (pMMR) colorectal cancer, the colorectal cancer may be a stage 0 colon cancer, a stage I colon cancer, a stage II colon cancer, a stage III colon cancer, or a stage IV colon cancer.

Other embodiments will be apparent from the following specification.

### BRIEF DESCRIPTION OF THE DRAWINGS

The application file contains at least one drawing executed in color. Copies of this patent or patent application with color drawings will be provided by the Office upon request and payment of the necessary fee.

FIGS. 1A and 1B are CONSORT diagrams showing patient and sample flow for (A) BIOGRID 1 and (B) BIOGRID 2. Abbreviations are as follows: CT, chemotherapy; dMMR, defective mismatch repair; IHC, immunohistochemistry; MACC1, Metastasis Associated in Colon Cancer 1; pMMR, proficient mismatch repair; PCR, quantitative real-time polymerase chain reaction.

FIGS. 2A-2C are Kaplan-Meier curves of recurrence-free survival (RFS) of patients in the Charité 1 cohort based on analysis of cryo-conserved tissues for microsatellite instability (MSI) status and MACC1 expression level. FIG. 2A includes chemotherapy-naïve patients stratified on the basis of MSI status. FIG. 2B includes chemotherapy-naïve patients stratified on the basis of tumor MACC1 expression level by qRT-PCR. FIG. 2C includes chemotherapy-naïve patients stratified on the basis of tumor MSI status and MACC1 expression level by qRT-PCR.

FIGS. 3A-3H are Kaplan-Meier curves of relapse-free survival of the Charité 2 cohort using either cryo-preserved- or FFPE-tissue to stratify on the basis of MACC1 expression in combination with one or both of the following housekeeping genes for normalization: glucose-6-phosphate-dehydrogenase (G6PD), or hypoxanthine phosphoribosyltransferase1 (HPRT1).

FIGS. 4A-4G are Kaplan-Meier curves of recurrence-free survival (RFS) of patients in the BIOGRID 1 training cohort based on analysis of FFPE tissues for MMR status and MACC1 expression level in chemotherapy-naïve and chemotherapy-treated patients with stage II T3/T4 colon cancer. FIG. 4A includes chemotherapy-naïve patients stratified on the basis of Tumor MMR status. FIG. 4B includes chemotherapy-naïve patients stratified on the basis of tumor MACC1 expression level by qRT-PCR. FIG. 4C includes chemotherapy-naïve patients stratified on the basis of tumor MACC1 expression level by IHC. FIG. 4D includes chemotherapy-naïve patients stratified on the basis of MMR status and MACC1 mRNA expression. FIG. 4E includes chemotherapy-naïve patients stratified on the basis of MMR status and MACC1 protein expression. FIG. 4F includes chemotherapy-treated (5-fluorouracil) patients stratified on the basis of MMR status and MACC1 mRNA expression. FIG. 4G includes chemotherapy-treated (5-fluorouracil) patients stratified on the basis of MMR status and MACC1 protein expression.

FIGS. 5A-5G are Kaplan-Meier curves of recurrence-free survival (RFS) of patients in the BIOGRID 1 training cohort based on analysis of FFPE tissues for MMR status and MACC1 expression level in chemotherapy-naïve and chemotherapy-treated patients with stage II T3 colon cancer. FIG. 5A includes chemotherapy-naïve patients stratified on the basis of Tumor MMR status. FIG. 5B includes chemotherapy-naïve patients stratified on the basis of tumor MACC1 expression level by qRT-PCR. FIG. 5C includes chemotherapy-naïve patients stratified on the basis of tumor MACC1 expression level by IHC. FIG. 5D includes chemotherapy-naïve patients stratified on the basis of MMR status and MACC1 mRNA expression. FIG. 5E includes chemotherapy-naïve patients stratified on the basis of MMR status and MACC1 protein expression. FIG. 5F includes chemotherapy-treated (5-fluorouracil) patients stratified on the basis of MMR status and MACC1 mRNA expression. FIG. 5G includes chemotherapy-treated (5-fluorouracil) patients stratified on the basis of MMR status and MACC1 protein expression.

FIGS. 6A-6C are Kaplan-Meier curves of patients in the BIOGRID 2 validation cohort analysis based on analysis of FFPE tissues for the combination of MMR status and MACC1 protein expression in chemotherapy-naïve and -treated patients with stage II T3/T4 colorectal cancer. FIG. 6A illustrates RFS discriminated by tumor MMR status in chemotherapy-naïve patients. FIG. 6B illustrates RFS discriminated by MACC1 protein expression levels quantified by IHC scoring in chemotherapy-naïve patients. FIG. 6C illustrates RFS discriminated by a combination of MMR status and MACC1 protein expression in chemotherapy-naïve patients.

FIGS. 7A-7C are Kaplan-Meier curves of patients in the BIOGRID 2 validation cohort analysis based on analysis of FFPE tissues for the combination of MMR status and MACC1 protein expression in chemotherapy-naïve and -treated patients with stage II T3 colorectal cancer. FIG. 7A illustrates RFS discriminated by tumor MMR status in chemotherapy-naïve patients. FIG. 7B illustrates RFS discriminated by MACC1 protein expression levels quantified by IHC scoring in chemotherapy-naïve patients. FIG. 7C illustrates RFS discriminated by a combination of MMR status and MACC1 protein expression in chemotherapy-naïve patients.

FIG. 8 is a Kaplan-Meier curve of patients in the pooled BIOGRID 1 and BIOGRID 2 cohorts.

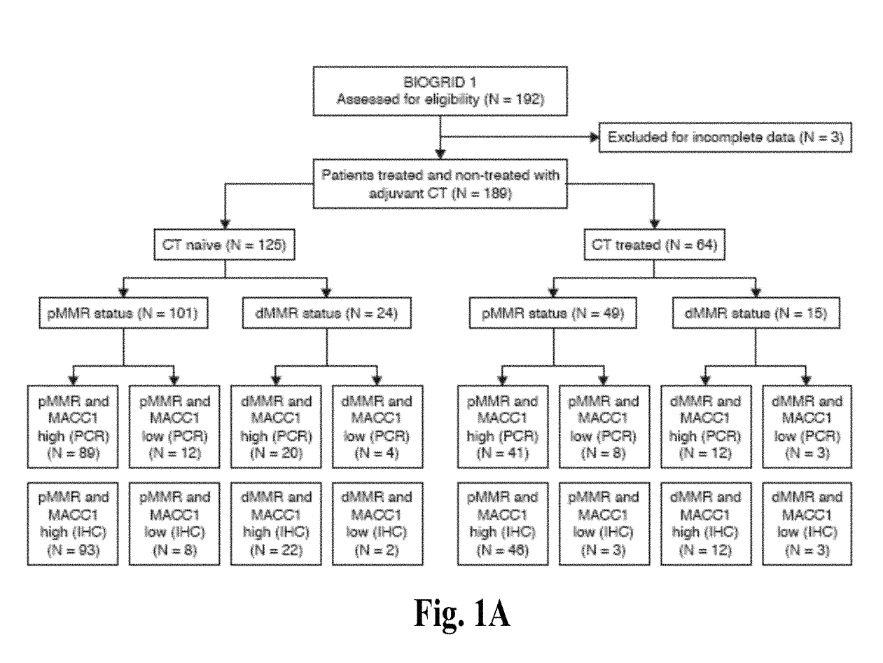

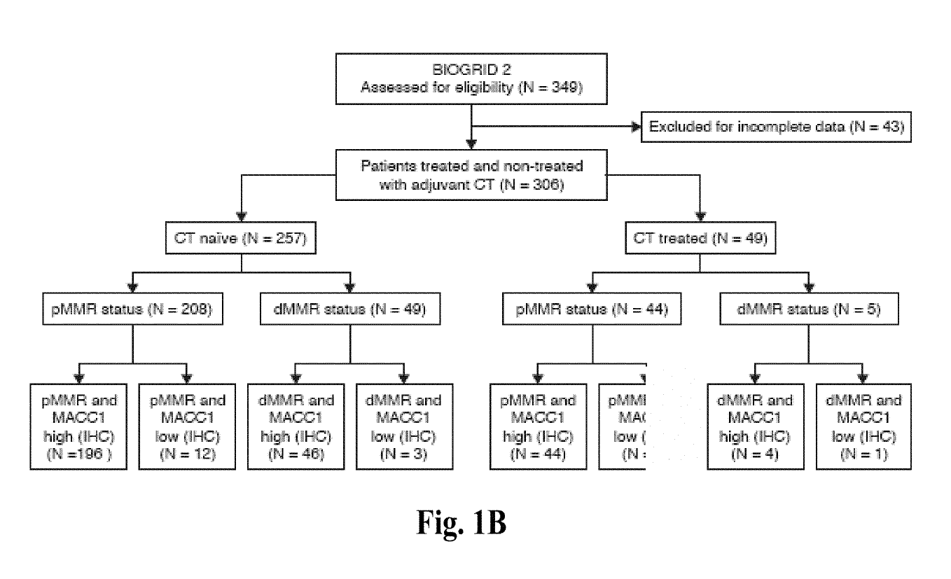

### DETAILED DESCRIPTION OF THE INVENTION

### I. Introduction

Expression of Metastasis Associated in Colon Cancer 1 (MACC1) was previously identified as a prognostic marker for colorectal cancers. See U.S. Pat. No. 7,851,168. We have discovered that a combination of mismatch repair (MMR) and MACC1 gene expression status of the patient could serve as a basis for risk stratification of early stage colon cancer patients. Patients with defective MMR (dMMR) status have improved survival and do not benefit from 5-fluorouracil (5-FU) therapies. (Sargent et al. 2010; Ribic et al. 2003; Popat et al. 2005). In contrast, patients with a proficient MMR (pMMR) status have a higher risk of recurrence and worse survival. The pMMR patients are then further stratified on the basis of MACC1 gene expression. Patients with a pMMR status and a low MACC1 expression have a favorable prognosis similar to patients having a dMMR status, whereas patients having a pMMR status and high MACC1 expression have a less favorable prognosis.

Therefore, the present invention relates generally to prognosing subjects having a pMMR status by quantifying expression level of a MACC1 gene product. A high level of MACC1 correlates with a unfavorable prognosis, which patients are most likely to benefit from receipt of a chemotherapy. A low level of MACC1 correlates with a favorable prognosis (comparable to dMMR), which patients are less likely to benefit from receipt of a chemotherapy.

The MACC1 gene product may be protein or a nucleic acid (such as a mRNA). In an embodiment, MACC1 protein is detected and quantitated histochemically, for example, by counting the number of tumor cells expressing MACC1 protein. In another embodiment, MACC1 mRNA is detected and quantitated by performing reverse transcription followed by quantitative PCR on the resulting cDNA molecules.

In an embodiment, the methods include characterizing a naïve sample for MMR status by contacting the sample with a panel of biomarker-specific reagents for detecting a set of gene products including a MLH1 gene product, a MSH2 gene product, a MSH6 gene product, and a PMS2 gene product. The gene products may be protein or mRNA for each of the MMR biomarkers.

Kits are also provided for performing the methods described herein.

The presently described assays rely on proven, widely adopted technology and provide accurate, reproducible, and rapid results.

### II. Definitions

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, or a combination thereof.

As used herein, the term “biomarker” shall refer to any molecule or group of molecules found in a biological sample that can be used to characterize the biological sample or a subject from which the biological sample is obtained. For example, a biomarker may be a molecule or group of molecules whose presence, absence, or relative abundance is:
characteristic of a particular disease state;
indicative of the severity of a disease or the likelihood of disease progression or regression; and/or
predictive that a particular disease state will respond to a particular treatment.
   As another example, the biomarker may be an infectious agent (such as a bacterium, fungus, virus, or other microorganism), or a substituent molecule or group of molecules thereof.

As used herein, the terms “sample” and “biological sample” shall refer to any composition containing or presumed to contain a biomarker. The term includes purified or separated components of cells, tissues, or blood, e.g., DNA, RNA, proteins, cell-free portions, or cell lysates. The sample can be a formalin-fixed, paraffin-embedded (FFPE) cellular sample, e.g., from a tumor or metastatic lesion. The sample can also be from frozen or fresh tissue, or from a liquid sample, e.g., blood or a blood component (plasma or serum), urine, semen, saliva, sputum, mucus, semen, tear, lymph, cerebral spinal fluid, material washed from a swab, etc. Samples also may include constituents and components of in vitro cultures of cells obtained from an individual, including cell lines. The sample can also be partially processed from a sample directly obtained from an individual, e.g., cell lysate or blood depleted of red blood cells.

As used herein, the term “cellular sample” refers to any sample containing intact cells, such as cell cultures, bodily fluid samples or surgical specimens taken for pathological, histological, or cytological interpretation.

As used herein, the term “tissue sample” shall refer to a cellular sample that preserves the cross-sectional spatial relationship between the cells as they existed within the subject from which the sample was obtained. “Tissue sample” shall encompass both primary tissue samples (i.e. cells and tissues produced by the subject) and xenografts (i.e. foreign cellular samples implanted into a subject).

As used herein, the term “cytological sample” refers to a cellular sample in which the cells of the sample have been partially or completely disaggregated, such that the sample no longer reflects the spatial relationship of the cells as they existed in the subject from which the cellular sample was obtained. Examples of cytological samples include tissue scrapings (such as a cervical scraping), fine needle aspirates, samples obtained by lavage of a subject, et cetera.

As used herein, “histochemical detection” refers to a process involving labelling a biomarker or other structures in a tissue sample with detection reagents in a manner that permits microscopic detection of the biomarker or other structures in the context of the cross-sectional relationship between the structures of the tissue sample. Examples include immunohistochemistry (IHC), chromogenic in situ hybridization (CISH), fluorescent in situ hybridization (FISH), silver in situ hybridization (SISH), and hematoxylin and eosin (H&E) staining of formalin-fixed, paraffin-embedded tissue sections.

As used herein, “cytochemical detection” refers to a process involving labelling a biomarker or other structures in a cytological sample with detection reagents in a manner that permits microscopic detection of the biomarker or other structures in the context of the cells of the cytological sample.

As used herein, the term “section” shall refer to a thin slice of a tissue sample suitable for microscopic analysis, typically cut using a microtome.

As used herein, the term “serial section” shall refer to any one of a series of sections cut in sequence from a tissue sample. For two sections to be considered “serial sections” of one another, they do not necessarily need to consecutive sections from the tissue, but they should generally contain the same tissue structures in the same cross-sectional relationship, such that the structures can be matched to one another after histological staining.

As used herein, the phrase “specific binding,” “specifically binds to,” or “specific for” refers to measurable and reproducible interactions such as binding between a target and a biomarker-specific agent, which is determinative of the presence of the target in the presence of a heterogeneous population of molecules including biological molecules. For example, a binding entity that specifically binds to a target is an antibody that binds this target with greater affinity, avidity, more readily, and/or with greater duration than it binds to other targets. In one embodiment, the extent of binding of a binding entity to an unrelated target is less than about 10% of the binding of the antibody to the target as measured, e.g., by a radioimmunoassay (RIA). In certain embodiments, a binding entity that specifically binds to a target has a dissociation constant (Kd) of ≤1 μM, ≤100 nM, ≤10 nM, ≤1 nM, or ≤0.1 nM. In another embodiment, specific binding can include, but does not require exclusive binding.

As used herein, the term “biomarker-specific agent” shall refer to any compound or composition that binds to a biomarker or a specific structure within that biomarker in a manner that permits detection of the biomarker in a sample. Examples include:
nucleic acid probes capable of specifically hybridizing to particular nucleotide sequences;
nucleic acid primer sets capable of amplifying a specific nucleotide sequence or set of sequences when paired with appropriate amplification reagents;
antibodies and antigen binding fragments thereof; and
engineered specific binding structures, including ADNECTINs (scaffold based on 10th FN3 fibronectin; Bristol-Myers-Squibb Co.), AFFIBODYs (scaffold based on Z domain of protein A from S. aureus; Affibody AB, Solna, Sweden), AVIMERs (scaffold based on domain A/LDL receptor; Amgen, Thousand Oaks, Calif.), dAbs (scaffold based on VH or VL antibody domain; GlaxoSmithKline PLC, Cambridge, UK), DARPins (scaffold based on Ankyrin repeat proteins; Molecular Partners AG, Zürich, C H), ANTICALINs (scaffold based on lipocalins; Pieris A G, Freising, D E), NANOBODYs (scaffold based on VHH (camelid Ig); Ablynx N/V, Ghent, B E), TRANS-BODYs (scaffold based on Transferrin; Pfizer Inc., New York, N.Y.), SMIPs (Emergent Biosolutions, Inc., Rockville, Md.), and TETRANECTINs (scaffold based on C-type lectin domain (CTLD), tetranectin; Borean Pharma A/S, Aarhus, D K) (Descriptions of such engineered specific binding structures are reviewed by Wurch et al., Development of Novel Protein Scaffolds as Alternatives to Whole Antibodies for Imaging and Therapy: Status on Discovery Research and Clinical Validation, Current Pharmaceutical Biotechnology, Vol. 9, pp. 502-509 (2008), the content of which is incorporated by reference).

A “detection reagent” when used in connection with a histochemical assay (including immunohistochemistry and in situ hybridization) is any reagent that is used to deposit a stain in proximity to a biomarker-specific agent bound to a biomarker in a cellular sample. Non-limiting examples include secondary antibodies capable of binding to a biomarker-specific antibody; enzymes linked to such secondary antibodies; and chemicals reactive with such enzymes to effect deposition of a fluorescent or chromogenic stain; and the like.

When used as a noun, the term “stain” shall refer to any substance that can be used to visualize specific molecules or structures in a cellular sample for microscopic analysis, including brightfield microscopy, fluorescent microscopy, electron microscopy, and the like. When used as a verb, the term “stain” shall refer to any process that results in deposition of a stain on a cellular sample.

The term “multiplex” refers to an assay in which more than one target is detected.

When used in the context of histochemical detection, a “multiplex stain” shall refer to histochemical staining method in which multiple biomarker-specific agents that bind to different biomarkers are applied to a single section of a tissue sample, and the different biomarkers are individually detected.

The terms “receptacle,” “vessel,” “tube,” “well,” “chamber,” “microchamber,” etc. refer to a container that can hold reagents or an assay. If the receptacle is in a kit and holds reagents, or is being used for an amplification reaction, it can be closed or sealed to avoid contamination or evaporation. If the receptacle is being used for an assay, it can be open or accessible, at least during set up of the assay.

The terms “individually detected” or “individual detection,” referring to a marker gene or marker gene product, indicates that each marker in a multiplex reaction is detected. That is, each marker is associated with a different label (detected by a differently labeled probe).

The terms “level of expression” or “expression level” in general are used interchangeably and generally refer to the amount of a polynucleotide, mRNA, or an amino acid product or protein in a biological sample. “Expression” generally refers to the process by which gene-encoded information is converted into the structures present and operating in the cell. Therefore, according to the invention “expression” of a gene may refer to transcription into a polynucleotide, translation into a protein, or even posttranslational modification of the protein. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. In some embodiments, “expression level” refers to amount of a protein in a biological sample as determined using immunohistochemistry (IHC), immunoblotting (e.g., Western blotting), immunofluorescence (IF), Enzyme-Linked Immunosorbant Assay (ELISA), or flow cytometry. In some embodiments, “expression level” refers to amount of a mRNA in a biological sample as determined using a reverse transcription and a quantitative PCR reaction.

The terms “nucleic acid,” “polynucleotide,” and “oligonucleotide” refer to polymers of nucleotides (e.g., ribonucleotides or deoxyribo-nucleotides) and includes naturally-occurring (adenosine, guanidine, cytosine, uracil and thymidine), non-naturally occurring, and modified nucleic acids. The term is not limited by length (e.g., number of monomers) of the polymer. A nucleic acid may be single-stranded or double-stranded and will generally contain 5′-3′ phosphodiester bonds, although in some cases, nucleotide analogs may have other linkages. Monomers are typically referred to as nucleotides. The term “non-natural nucleotide” or “modified nucleotide” refers to a nucleotide that contains a modified nitrogenous base, sugar or phosphate group, or that incorporates a non-natural moiety in its structure. Examples of non-natural nucleotides include dideoxynucleotides, biotinylated, aminated, deaminated, alkylated, benzylated and fluorophor-labeled nucleotides.

The term “primer” refers to a short nucleic acid (an oligonucleotide) that acts as a point of initiation of polynucleotide strand synthesis by a nucleic acid polymerase under suitable conditions. Polynucleotide synthesis and amplification reactions typically include an appropriate buffer, dNTPs and/or rNTPs, and one or more optional cofactors, and are carried out at a suitable temperature. A primer typically includes at least one target-hybridized region that is at least substantially complementary to the target sequence (e.g., having 0, 1, 2, or 3 mismatches). This region of is typically about 8 to about 40 nucleotides in length, e.g., 12-25 nucleotides. A “primer pair” refers to a forward and reverse primer that are oriented in opposite directions relative to the target sequence, and that produce an amplification product in amplification conditions. In some embodiments, multiple primer pairs rely on a single common forward or reverse primer. For example, multiple allele-specific forward primers can be considered part of a primer pair with the same, common reverse primer, e.g., if the multiple alleles are in close proximity to each other.

As used herein, “probe” means any molecule that is capable of selectively binding to a specifically intended target biomolecule, for example, a nucleic acid sequence of interest that hybridizes to the probes. The probe is detectably labeled with at least one non-nucleotide moiety. In some embodiments, the probe is labeled with a fluorophore and quencher.

The words “complementary” or “complementarity” refer to the ability of a nucleic acid in a polynucleotide to form a base pair with another nucleic acid in a second polynucleotide. For example, the sequence A-G-T (A-G-U for RNA) is complementary to the sequence T-C-A (U-C-A for RNA). Complementarity may be partial, in which only some of the nucleic acids match according to base pairing, or complete, where all the nucleic acids match according to base pairing. A probe or primer is considered “specific for” a target sequence if it is at least partially complementary to the target sequence. Depending on the conditions, the degree of complementarity to the target sequence is typically higher for a shorter nucleic acid such as a primer (e.g., greater than 80%, 90%, 95%, or higher) than for a longer sequence. In some embodiments, the term “each primer pair specific for a different sequence in the gene” indicates that each primer pair specifically amplifies a different sequence, e.g., a different allele or mutation, of the respective gene.

The term “specifically amplifies” indicates that a primer set amplifies a target sequence more than non-target sequence at a statistically significant level. The term “specifically detects” in the context of an amplification reaction indicates that a probe will detect a target sequence more than non-target sequence at a statistically significant level. As will be understood in the art, specific amplification and detection can be determined using a negative control, e.g., a sample that includes the same nucleic acids as the test sample, but not the target sequence or a sample lacking nucleic acids. For example, primers and probes that specifically amplify and detect a target sequence result in a Ct that is readily distinguishable from background (non-target sequence), e.g., a Ct that is at least 2, 3, 4, 5, 5-10, 10-20, or 10-30 cycles less than background. The term “allele-specific” PCR refers to amplification of a target sequence using primers that specifically amplify a particular allelic variant of the target sequence. Typically, the forward or reverse primer includes the exact complement of the allelic variant at that position.

The terms “identical” or “percent identity,” in the context of two or more nucleic acids, or two or more polypeptides, refer to two or more sequences or subsequences that are the same or have a specified percentage of nucleotides, or amino acids, that are the same (e.g., about 60% identity, e.g., at least any of 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters, or by manual alignment and visual inspection. See e.g., the NCBI web site at ncbi.nlm.nih.gov/BLAST. Such sequences are then said to be “substantially identical.” Percent identity is typically determined over optimally aligned sequences, so that the definition applies to sequences that have deletions and/or additions, as well as those that have substitutions. The algorithms commonly used in the art account for gaps and the like. Typically, identity exists over a region comprising an a sequence that is at least about 8-25 amino acids or nucleotides in length, or over a region that is 50-100 amino acids or nucleotides in length, or over the entire length of the reference sequence.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “kit” refers to any manufacture (e.g., a package or a container) including at least one reagent, such as a biomarker-specific agent, nucleic acid probe or probe pool or the like, for specifically amplifying, capturing, tagging/converting or otherwise detecting a biomarker or group of biomarkers.

The term “amplification conditions” refers to conditions in a nucleic acid amplification reaction (e.g., PCR amplification) that allow for hybridization and template-dependent extension of the primers. The term “amplicon” or “amplification product” refers to a nucleic acid molecule that contains all or a fragment of the target nucleic acid sequence and that is formed as the product of in vitro amplification by any suitable amplification method. One of skill will understand that a forward and reverse primer (primer pair) defines the borders of an amplification product. The term “generate an amplification product” when applied to primers, indicates that the primers, under appropriate conditions (e.g., in the presence of a nucleotide polymerase and NTPs), will produce the defined amplification product. Various PCR conditions are described in PCR Strategies (Innis et al., 1995, Academic Press, San Diego, Calif.) at Chapter 14; PCR Protocols: A Guide to Methods and Applications (Innis et al., Academic Press, N Y, 1990)

The term “amplification product” refers to the product of an amplification reaction. The amplification product includes the primers used to initiate each round of polynucleotide synthesis. An “amplicon” is the sequence targeted for amplification, and the term can also be used to refer to amplification product. The 5′ and 3′ borders of the amplicon are defined by the forward and reverse primers.

The terms “individual”, “subject”, and “patient” are used interchangeably herein. The individual can be pre-diagnosis, post-diagnosis but pre-therapy, undergoing therapy, or post-therapy. In the context of the present disclosure, the individual is typically seeking medical or veterinary care.

The term “obtaining a sample from an individual” means that a biological sample from the individual is provided for testing. The obtaining can be directly from the individual, or from a third party that directly obtained the sample from the individual.

The term “providing therapy for an individual” means that the therapy is prescribed, recommended, or made available to the individual. The therapy may be actually administered to the individual by a third party (e.g., an in-patient injection), or by the individual himself.

A “control” sample or value refers to a value that serves as a reference, usually a known reference, for comparison to a test sample or test conditions. For example, a test sample can be taken from a test condition, e.g., from an individual suspected of having cancer, and compared to samples from known conditions, e.g., from a cancer-free individual (negative control), or from an individual known to have cancer (positive control). In the context of the present disclosure, the test sample is typically from a breast cancer patient. A control can also represent an average value or a range gathered from a number of tests or results. A control can also be prepared for reaction conditions. For example, a control for the presence, quality, and/or quantity of nucleic acid (e.g., internal control) can include primers or probes that will detect a sequence known to be present in the sample (e.g., a housekeeping gene such as beta actin, beta globin, glyceraldehyde 3-phosphate dehydrogenase (GAPDH), ribosomal protein L37 and L38, PPIase, EIF3, eukaryotic translation elongation factor 2 (eEF2), DHFR, succinate dehydrogenase, Glucose-6-phosphate-dehydrogenase (G6PD), or hypoxanthine phosphoribosyltransferase1 (HPRT)). In some embodiments, the internal control can be a sequence from a region of the same gene that is not commonly variant (e.g., in a different exon). A known added polynucleotide, e.g., having a designated length, can also be added. An example of a negative control is one free of nucleic acids, or one including primers or probes specific for a sequence that would not be present in the sample, e.g., from a different species. One of skill will understand that the selection of controls will depend on the particular assay, e.g., so that the control is cell type and organism-appropriate. One of skill in the art will recognize that controls can be designed for assessment of any number of parameters. For example, a control can be devised to compare therapeutic benefit based on pharmacological data (e.g., half-life) or therapeutic measures (e.g., comparison of benefit and/or side effects). Controls can be designed for in vitro applications. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The terms “label,” “tag,” “detectable moiety,” and like terms refer to a composition detectable by spectroscopic, photochemical, biochemical, immunochemical, chemical, or other physical means. For example, useful labels include fluorescent dyes (fluorophores), luminescent agents, radioisotopes (e.g., ³²P, ³H), electron-dense reagents, or an affinity-based moiety, e.g., a poly-A (interacts with poly-T) or poly-T tag (interacts with poly-A), a His tag (interacts with Ni), or a strepavidin tag (separable with biotin). One of skill will understand that a detectable label conjugated to a nucleic acid or a protein is not naturally occurring.

The term “favorable prognosis” refers to a patient with a colorectal cancer whose cancer is less likely to recur or progress within 3 years after primary surgical resection (with curative intent).

The term “unfavorable prognosis” refers to a patient with a colorectal cancer whose cancer is more likely to recur or progress within 3 years after primary surgical resection (with curative intent).

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4th ed. 2007); Sambrook et al., MOLECULAR CLONING, A LABORATORY MANUAL, Cold Springs Harbor Press (Cold Springs Harbor, N.Y. 1989). The term “a” or “an” is intended to mean “one or more.” The terms “comprise,” “comprises,” and “comprising,” when preceding the recitation of a step or an element, are intended to mean that the addition of further steps or elements is optional and not excluded.

### III. Biomarker Descriptions

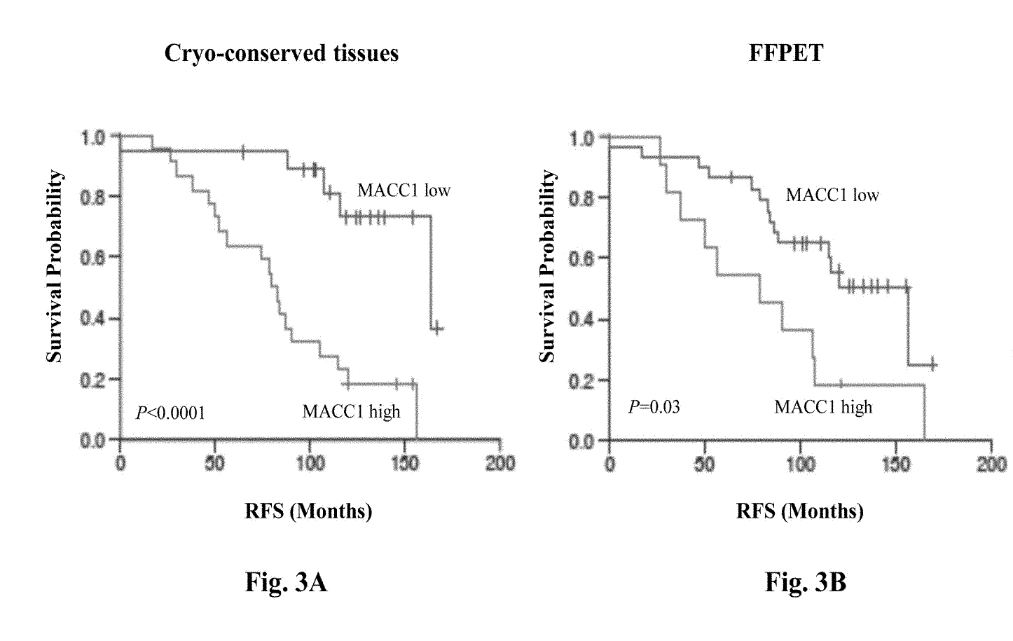

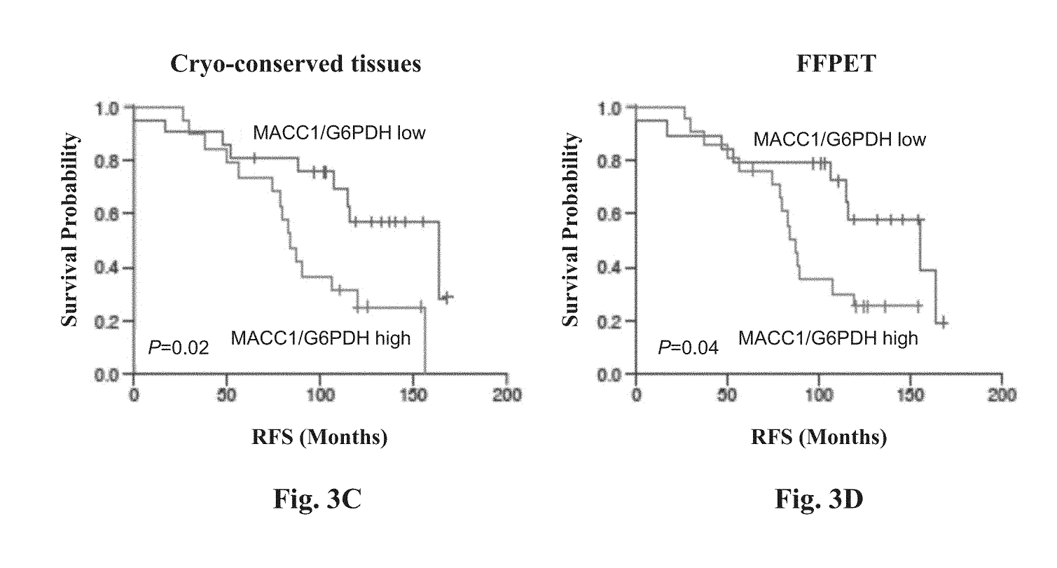

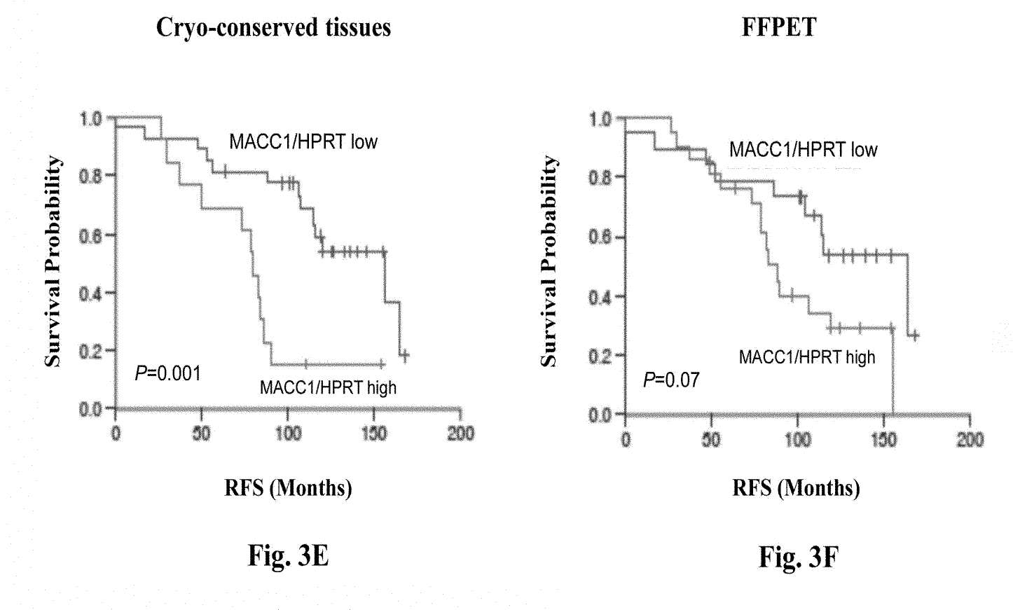

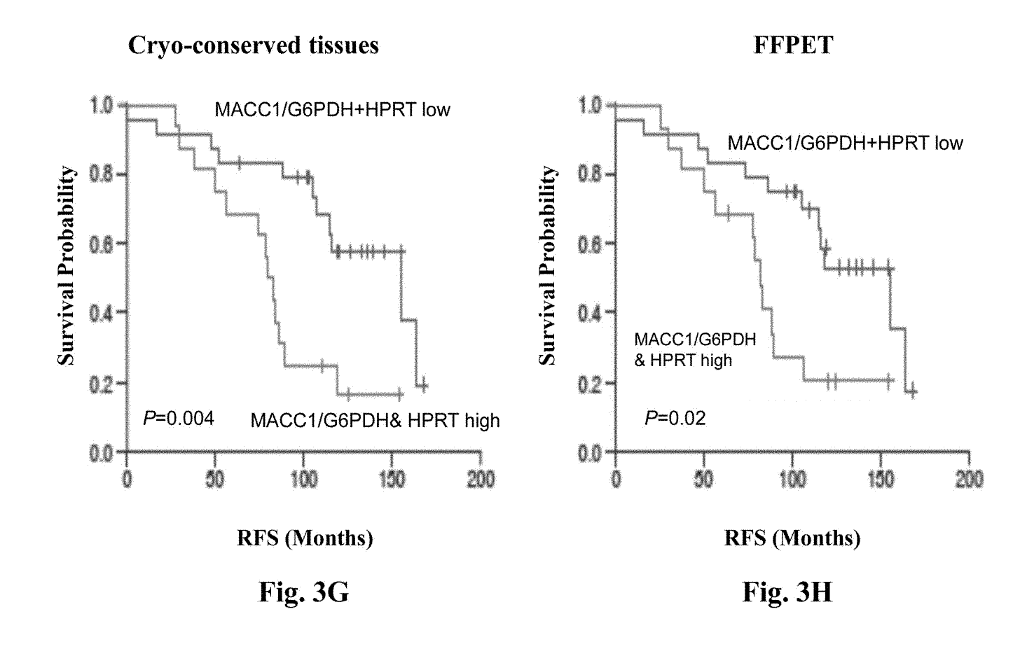

MACC1: The human MACC1 gene is located on chromosome 7, at location 7p21.1. The canonical sequence of the gene encodes a coding sequence 2,559 nucleotides in length that is transcribed to a 852-amino acid protein that acts as a transcription activator for MET and as a key regulator of HGF-MET signaling. MACC1 protein (also known as Metastasis-associated in colon cancer protein 1 and SH3 domain-containing protein 7a5) has been shown to promote cell motility, proliferation and hepatocyte growth factor (HGF)-dependent scattering in vitro and tumor growth and metastasis in vivo. The canonical protein sequence for MACC1 protein, as well as several known variants thereof, are disclosed at Uniprot Accession Number Q6ZN28. As used herein, the term “human MACC1 protein” encompasses the canonical sequence and natural variants thereof that maintain the function of the canonical sequence. In some embodiments, a MACC1 protein-specific agent encompasses biomarker-specific agent that is capable of specifically binding to the canonical MACC1 protein sequence, such as the sequence of SEQ ID NO: 14. The term “MACC1 nucleic acid” shall refer to any nucleic acid sequence encoding a MACC1 protein (such as a protein having an amino acid sequence of SEQ ID NO: 14). An exemplary coding sequence of a nucleic acid can be found at, for example, FIG. 3 of U.S. Pat. No. 7,851,168 (reproduced herein at SEQ ID NO: 18) and European Nucleotide Archive Accession No. BC137090.1 (available athttp://www.ebi.ac.uk/ena/data/view/BC137090). In some embodiments, a MACC1 nucleic acid-specific agent encompasses biomarker-specific agents that are substantially complementary to the exemplary coding sequence of European Nucleotide Archive Accession No. BC137090.1, to SEQ ID NO: 18, to a nucleic acid encoding SEQ ID NO: 14, or to a complement thereof. In some embodiments, a MACC1 nucleic acid-specific agent encompasses biomarker-specific agents that are substantially complementary to the exemplary coding sequence of European Nucleotide Archive Accession No. BC137090.1, to SEQ ID NO: 18, to the nucleic acid encoding SEQ ID NO: 14, or to the complement thereof, and that, when reacted with an appropriate set of reagents, results in the amplification of a portion of the exemplary coding sequence of European Nucleotide Archive Accession No. BC137090.1, of SEQ ID NO: 18, or of the nucleic acid encoding SEQ ID NO: 14.

MLH1: The human MLH1 gene is located on chromosome 3, at location 3p21.3. The canonical sequence of the gene encodes a coding sequence 9,347 nucleotides in length that is transcribed to a 756-amino acid protein. MLH1 protein heterodimerizes with PMS2 to form MutL alpha, a component of the post-replicative DNA mismatch repair system (MMR). DNA repair is initiated by MutS alpha (MSH2-MSH6) or MutS beta (MSH2-MSH6) binding to a dsDNA mismatch, then MutL alpha is recruited to the heteroduplex. Assembly of the MutL-MutS-heteroduplex ternary complex in presence of RFC and PCNA is sufficient to activate endonuclease activity of PMS2. It introduces single-strand breaks near the mismatch and thus generates new entry points for the exonuclease EXO1 to degrade the strand containing the mismatch. DNA methylation would prevent cleavage and therefore assure that only the newly mutated DNA strand is going to be corrected. The canonical protein sequence for MLH1 protein, as well as several known variants thereof, are disclosed at Uniprot Accession Number P40692. As used herein, the term “human MLH1 protein” encompasses the canonical sequence and natural variants thereof that maintain the function of the canonical sequence. In some embodiments, a MLH1 protein-specific agent encompasses biomarker-specific agent that is capable of specifically binding to the canonical MLH1 protein sequence as disclosed at Uniprot Accession Number P40692, such as the sequence disclosed at SEQ ID NO: 15. The term “MLH1 nucleic acid” shall refer to any nucleic acid sequence encoding a MLH1 protein. An exemplary coding sequence of a nucleic acid can be found at, for example, the sequence disclosed at SEQ ID NO: 24 and at European Nucleotide Archive Accession No. AH003234.2 (available at http://www.ebi.ac.uk/ena/data/view/AH003234). In some embodiments, a MLH1 nucleic acid-specific agent encompasses biomarker-specific agents that are substantially complementary to the exemplary coding sequence of European Nucleotide Archive Accession No. AH003234.2, to SEQ ID NO: 24, to a nucleic acid encoding SEQ ID NO: 15, or to a complement thereof. In some embodiments, a MLH1 nucleic acid-specific agent encompasses biomarker-specific agents that: (1) are substantially complementary to the exemplary coding sequence of European Nucleotide Archive Accession No. AH003234.2, to SEQ ID NO: 24, to a nucleic acid encoding SEQ ID NO: 15, or to a complement thereof; and (2) when reacted with an appropriate set of reagents, results in the amplification of a portion of the exemplary coding sequence of European Nucleotide Archive Accession No. AH003234.2, of SEQ ID NO: 24, or of the nucleic acid encoding SEQ ID NO: 15.

MSH2: The human MSH2 gene is located on chromosome 2, at location 2p21. The canonical sequence of the gene encodes a coding sequence 3,080 nucleotides in length that is transcribed to a 934-amino acid protein. Component of the post-replicative DNA mismatch repair system (MMR). Forms two different heterodimers: MutS alpha (MSH2-MSH6 heterodimer) and MutS beta (MSH2-MSH3 heterodimer) which binds to DNA mismatches thereby initiating DNA repair. When bound, heterodimers bend the DNA helix and shields approximately 20 base pairs. MutS alpha recognizes single base mismatches and dinucleotide insertion-deletion loops (IDL) in the DNA. MutS beta recognizes larger insertion-deletion loops up to 13 nucleotides long. After mismatch binding, MutS alpha or beta forms a ternary complex with the MutL alpha heterodimer, which is thought to be responsible for directing the downstream MMR events, including strand discrimination, excision, and resynthesis. ATP binding and hydrolysis play a pivotal role in mismatch repair functions. The ATPase activity associated with MutS alpha regulates binding similar to a molecular switch: mismatched DNA provokes ADP-->ATP exchange, resulting in a discernible conformational transition that converts MutS alpha into a sliding clamp capable of hydrolysis-independent diffusion along the DNA backbone. This transition is crucial for mismatch repair. MutS alpha may also play a role in DNA homologous recombination repair. The canonical protein sequence for MSH2 protein, as well as several known variants thereof, are disclosed at Uniprot Accession Number P43246. As used herein, the term “human MSH2 protein” encompasses the canonical sequence and natural variants thereof that maintain the function of the canonical sequence. In some embodiments, a MSH2 protein-specific agent encompasses biomarker-specific agent that is capable of specifically binding to the canonical MSH2 protein sequence as disclosed at Uniprot Accession Number P43246, and as disclosed herein at SEQ ID NO: 16. The term “MSH2 nucleic acid” shall refer to any nucleic acid sequence encoding a MSH2 protein. An exemplary coding sequence of a nucleic acid can be found at, for example, European Nucleotide Archive Accession No. U03911.1 (available at http://www.ebi.ac.uk/ena/data/view/U03911) and at the sequence disclosed at SEQ ID NO: 25. In some embodiments, a MSH2 nucleic acid-specific agent encompasses biomarker-specific agents that are substantially complementary to the exemplary coding sequence of European Nucleotide Archive Accession No. U03911.1, to SEQ ID NO: 25, to a nucleic acid encoding SEQ ID NO: 16, or to a complement thereof. In some embodiments, a MLH1 nucleic acid-specific agent encompasses biomarker-specific agents that: (1) are substantially complementary to the exemplary coding sequence of European Nucleotide Archive Accession No. U03911.1, to SEQ ID NO: 25, to a nucleic acid encoding SEQ ID NO: 16, or to a complement thereof; and (2) when reacted with an appropriate set of reagents, results in the amplification of a portion of the exemplary coding sequence of European Nucleotide Archive Accession No. U03911.1, of SEQ ID NO: 25, or of the nucleic acid encoding SEQ ID NO: 16.

MSH6: The human MSH6 gene is located on chromosome 2, at location 2p16. The canonical sequence of the gene encodes a coding sequence 4,244 nucleotides in length that is transcribed to a 1360-amino acid protein. Component of the post-replicative DNA mismatch repair system (MMR). Heterodimerizes with MSH2 to form MutS alpha, which binds to DNA mismatches thereby initiating DNA repair. When bound, MutS alpha bends the DNA helix and shields approximately 20 base pairs, and recognizes single base mismatches and dinucleotide insertion-deletion loops (IDL) in the DNA. After mismatch binding, forms a ternary complex with the MutL alpha heterodimer, which is thought to be responsible for directing the downstream MMR events, including strand discrimination, excision, and resynthesis. ATP binding and hydrolysis play a pivotal role in mismatch repair functions. The ATPase activity associated with MutS alpha regulates binding similar to a molecular switch: mismatched DNA provokes ADP-->ATP exchange, resulting in a discernible conformational transition that converts MutS alpha into a sliding clamp capable of hydrolysis-independent diffusion along the DNA backbone. This transition is crucial for mismatch repair. MutS alpha may also play a role in DNA homologous recombination repair. Recruited on chromatin in G1 and early S phase via its PWWP domain that specifically binds trimethylated ‘Lys-36’ of histone H3 (H3K36me3): early recruitment to chromatin to be replicated allowing a quick identification of mismatch repair to initiate the DNA mismatch repair reaction. The canonical protein sequence for MSH6 protein, as well as several known variants thereof, are disclosed at Uniprot Accession Number P52701. As used herein, the term “human MSH6 protein” encompasses the canonical sequence and natural variants thereof that maintain the function of the canonical sequence. In some embodiments, a MSH6 protein-specific agent encompasses biomarker-specific agent that is capable of specifically binding to the canonical MSH6 protein sequence as disclosed at Uniprot Accession Number P52701, and as disclosed herein at SEQ ID NO: 17. The term “MSH6 nucleic acid” shall refer to any nucleic acid sequence encoding a MSH6 protein. An exemplary coding sequence of a nucleic acid can be found at, for example, European Nucleotide Archive Accession No. U54777.2 (available at http://www.ebi.ac.uk/ena/data/view/U54777), and at the sequence disclosed at SEQ ID NO: 26. In some embodiments, a MSH6 nucleic acid-specific agent encompasses biomarker-specific agents that are substantially complementary to the exemplary coding sequence of European Nucleotide Archive Accession No. U54777.2, to SEQ ID NO: 26, to a nucleic acid encoding SEQ ID NO: 17, or to a complement thereof. In some embodiments, a MSH6 nucleic acid-specific agent encompasses biomarker-specific agents that: (1) are substantially complementary to the exemplary coding sequence of European Nucleotide Archive Accession No. U54777.2, to SEQ ID NO: 26, to a nucleic acid encoding SEQ ID NO: 17, or to a complement thereof; and (2) when reacted with an appropriate set of reagents, results in the amplification of a portion of: the exemplary coding sequence of European Nucleotide Archive Accession No. U54777.2, SEQ ID NO: 26, or the nucleic acid encoding SEQ ID NO: 17.

PMS2: The human PMS2 gene is located on chromosome 2, at location 2p16. The gene encodes a coding sequence 2,589 nucleotides in length that is transcribed to an 862-amino acid protein. Component of the post-replicative DNA mismatch repair system (MMR). Heterodimerizes with MLH1 to form MutL alpha. DNA repair is initiated by MutS alpha (MSH2-MSH6) or MutS beta (MSH2-MSH6) binding to a dsDNA mismatch, then MutL alpha is recruited to the heteroduplex. Assembly of the MutL-MutS-heteroduplex ternary complex in presence of RFC and PCNA is sufficient to activate endonuclease activity of PMS2. It introduces single-strand breaks near the mismatch and thus generates new entry points for the exonuclease EXO1 to degrade the strand containing the mismatch. DNA methylation would prevent cleavage and therefore assure that only the newly mutated DNA strand is going to be corrected. MutL alpha (MLH1-PMS2) interacts physically with the clamp loader subunits of DNA polymerase III, suggesting that it may play a role to recruit the DNA polymerase III to the site of the MMR. Also implicated in DNA damage signaling, a process which induces cell cycle arrest and can lead to apoptosis in case of major DNA damages. The canonical protein sequence for PMS2 protein, as well as several known variants thereof, are disclosed at Uniprot Accession Number P54278. As used herein, the term “human PMS2 protein” encompasses the canonical sequence and natural variants thereof that maintain the function of the canonical sequence. In some embodiments, a PMS2 protein-specific agent encompasses biomarker-specific agent that is capable of specifically binding to the canonical PMS2 protein sequence as disclosed at Uniprot Accession Number P54278. The term “PMS2 nucleic acid” shall refer to any nucleic acid sequence encoding a PMS2 protein. An exemplary coding sequence of a nucleic acid can be found at, for example, NCBI Accession No. NM_000535.6 (available at http://www.ncbi.nlm.nih.gov/nuccore/NM_000535), and at the sequence disclosed at SEQ ID NO: 27. In some embodiments, a PMS2 nucleic acid-specific agent encompasses biomarker-specific agents that are substantially complementary to the exemplary coding sequence of NCBI Accession No. NM_000535.6, to SEQ ID NO: 27, to a nucleic acid encoding SEQ ID NO: 18, or to a complement thereof. In some embodiments, a PMS2 nucleic acid-specific agent encompasses biomarker-specific agents that: (1) are substantially complementary to the exemplary coding sequence of NCBI Accession No. NM_000535.6, to SEQ ID NO: 27, to a nucleic acid encoding SEQ ID NO: 18, or to a complement thereof; and (2) when reacted with an appropriate set of reagents, results in the amplification of a portion of: the exemplary coding sequence of NCBI Accession No. NM_000535.6, SEQ ID NO: 27, or the nucleic acid encoding SEQ ID NO: 18.

G6PD: The human G6PD gene is located on chromosome X, at location Xq28. The gene encodes at least two transcripts: (1) a 1638 nucleotide transcript (a consensus sequence for which can be found at the CCDS Database at accession number CCDS14756.2 (SEQ ID NO: 19)), which is translated to a 545 amino acid polypeptide; and (2) a 1548 nucleotide transcript (a consensus sequence for which can be found at the CCDS Database at accession number CCDS44023.1 (SEQ ID NO: 20)), which encodes a 515 amino acid polypeptide. The gene encodes Glucose-6-phosphate 1-dehydrogenase (also known as G6PD), which catalyzes the reaction of D-glucose 6-phosphate and NADP+ to 6-phospho-D-glucono-1,5-lactone and NADPH, the rate-limiting step of the oxidative pentose-phosphate pathway. The canonical protein sequence for G6PD protein, as well as several known variants thereof, are disclosed at Uniprot Accession Number P11413. As used herein, the term “human G6PD protein” encompasses the canonical sequence and natural variants thereof that maintain the function of the canonical sequence. The term “G6PD nucleic acid” shall refer to any nucleic acid sequence encoding a human G6PD protein (such as the amino acid sequence disclosed at SEQ ID NO: 21). Exemplary coding sequences of a nucleic acid can be found at, for example, SEQ ID NO: 19 or SEQ ID NO: 20. In some embodiments, a G6PD nucleic acid-specific agent encompasses biomarker-specific agents that are substantially complementary to SEQ ID NO: 19 or SEQ ID NO: 20 or to a nucleic acid encoding SEQ ID NO: 21. In some embodiments, a G6PD nucleic acid-specific agent encompasses biomarker-specific agents that are substantially complementary to SEQ ID NO: 19 or SEQ ID NO: 20 or to a nucleic acid encoding SEQ ID NO: 21 or a complement thereof, and that, when reacted with an appropriate set of reagents, results in the amplification of a portion of SEQ ID NO: 19 or SEQ ID NO: 20 or the nucleic acid encoding SEQ ID NO: 21.

HPRT: The human HPRT is located on the X chromosome at position Xq26.1. The gene encodes a canonical transcript of 657 nucleotides, which is translated to a 218 amino acid polypeptide. A consensus sequence for the transcript can be found at the CCDS Database at accession number CCDS14641.1 (SEQ ID NO: 22)). The protein encoded by the gene is Hypoxanthine-guanine phosphoribosyltransferase (also known as Hypoxanthine-guanine phosphoribosyltransferase (EC:2.4.2.8), HPRT1, HGPRT, and HGPRTase), which converts guanine to guanosine monophosphate, and hypoxanthine to inosine monophosphate by transfering the 5-phosphoribosyl group from 5-phosphoribosylpyrophosphate onto the purine. It plays a central role in the generation of purine nucleotides through the purine salvage pathway. The canonical protein sequence for HPRT, as well as several known variants thereof, are disclosed at Uniprot Accession Number P00492. As used herein, the term “human HPRT protein” encompasses the canonical sequence and natural variants thereof that maintain the function of the canonical sequence, such as the sequence disclosed. The term “HPRT nucleic acid” shall refer to any nucleic acid sequence encoding a human HPRT protein (such as the amino acid sequence of SEQ ID NO: 23). Exemplary coding sequences of a nucleic acid can be found at, for example, SEQ ID NO: 22. In some embodiments, a G6PD nucleic acid-specific agent encompasses biomarker-specific agents that are substantially complementary to SEQ ID NO: 22 or to a nucleic acid sequence encoding SEQ ID NO: 23, or a complement thereof. In some embodiments, a HPRT nucleic acid-specific agent encompasses biomarker-specific agents that are substantially complementary to SEQ ID NO: 22 or a nucleic acid sequence encoding SEQ ID NO: 23 or a complement thereof, and that, when reacted with an appropriate set of reagents, results in the amplification of a portion of SEQ ID NO: 22 or the nucleic acid sequence encoding SEQ ID NO: 23.

### IV. Nucleic Acid and Protein Samples

Samples for nucleic acid amplification can be obtained from any source suspected of containing nucleic acid. Samples can be taken from formalin fixed paraffin embedded tissue (FFPET), tissue biopsy, or cultured cells (e.g., obtained from a patient, or representing a control). In some embodiments, the sample is obtained in a non-invasive manner, e.g., from urine, skin, swab, saliva, blood or a blood fraction.

In a sample that includes cells, the cells can be separated out (e.g., using size-based filtration or centrifugation), thereby leaving cell free nucleic acids (cfNA), including nucleic acids in exosomes, microvesicles, viral particles, or those circulating freely. Alternatively, the cells can be lysed to obtain cellular nucleic acids, either in the presence of magnetic glass particles (MGPs) or before addition of the cellular lysate to the MGPs.

Methods for isolating nucleic acids from biological samples are known, e.g., as described in Sambrook, and several kits are commercially available (e.g., High Pure RNA Isolation Kit, High Pure Viral Nucleic Acid Kit, and MagNA Pure LC Total Nucleic Acid Isolation Kit, DNA Isolation Kit for Cells and Tissues, DNA Isolation Kit for Mammalian Blood, High Pure FFPET DNA Isolation Kit, available from Roche). In the context of the presently disclosed methods, RNA is collected, though in some embodiments, the classifier can be used on previously prepared cDNA.

In embodiments in which proteins are detected histochemically, the samples may be formalin-fixed paraffin embedded (FFPE) tissues samples.

### V. Protein Detection

Where the biomarker is a protein, any method of detecting and/or quantitating protein in a sample can be used, including, for example, immunohistochemistry (IHC), immunofluorescence (IF), immunoblotting (e.g., Western blotting), flow cytometry, and Enzyme-linked Immunosorbant Assay (ELIS A).

In a specific embodiment, the detection method comprises a histochemical staining procedure (such as immunohistochemistry or an analogous procedure using other entities specific for the protein biomarker). In certain embodiments, the biomarker specific reagents are deposited on serial sections using an automated slide stainer, such as a VENTANA BenchMark series IHC/ISH stainer, a Leica BOND series IHC/ISH stainer, a Dako AUTOSTAINER series IHC/ISH stainer, or the like. In a specific embodiment, the biomarker-specific agent is an antibody, and the biomarker-specific antibodies are deposited on formalin fixed, paraffin-embedded sections of the sample using an automated slide stainer.

Biomarker-specific reagents are visualized using detection reagents to deposit a detectable entity that generates a detectable signal associated with the biomarker. When associated with a biomarker-specific reagent (either directly or indirectly), the detectable signal can be used to locate and/or quantify the biomarker to which the biomarker-specific reagent is directed. Thereby, the presence and/or concentration of the target in a sample can be detected by detecting the signal produced by the detectable entity. A detectable signal can be generated by any mechanism including absorption, emission and/or scattering of a photon (including radio frequency, microwave frequency, infrared frequency, visible frequency and ultra-violet frequency photons). Detectable entities include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected through antibody-hapten binding interactions using additional detectably labeled antibody conjugates, and paramagnetic and magnetic molecules or materials. Particular examples of detectable entities include enzymes such as horseradish peroxidase, alkaline phosphatase, acid phosphatase, glucose oxidase, (3-galactosidase or 3-glucuronidase; fluorphores such as fluoresceins, luminophores, coumarins, BODIPY dyes, resorufins, and rhodamines (many additional examples of fluorescent molecules can be found in The Handbook—A Guide to Fluorescent Probes and Labeling Technologies, Molecular Probes, Eugene, Oreg.); nanoparticles such as quantum dots (obtained, for example, from QuantumDot Corp, Invitrogen Nanocrystal Technologies, Hayward, Calif.; see also, U.S. Pat. Nos. 6,815,064, 6,682,596 and 6,649,138, each of which patents is incorporated by reference herein); metal chelates such as DOTA and DPTA chelates of radioactive or paramagnetic metal ions like Gd 3+; and liposomes, for example, liposomes containing trapped fluorescent molecules. Where the detectable entity includes an enzyme, a detectable substrate such as a chromogen, a fluorogenic compound, or a luminogenic compound can be used in combination with the enzyme to generate a detectable signal (A wide variety of such compounds are commercially available, for example, from Invitrogen Corporation, Eugene Oreg.). Particular examples of chromogenic compounds include diaminobenzidine (DAB), 4-nitrophenylphospate (pNPP), fast red, bromochloroindolyl phosphate (BCIP), nitro blue tetrazolium (NBT), BCIP/NBT, fast red, AP Orange, AP blue, tetramethylbenzidine (TMB), 2,2′-azino-di-[3-ethylbenzothiazoline sulphonate] (ABTS), o-dianisidine, 4-chloronaphthol (4-CN), nitrophenyl-β-D-galactopyranoside (ONPG), o-phenylenediamine (OPD), 5-bromo-4-chloro-3-indolyl-β-galactopyranoside (X-Gal), methylumbelliferyl-β-D-galactopyranoside (MU-Gal), p-nitrophenyl-α-D-galactopyranoside (PNP), 5-bromo-4-chloro-3-indolyl-β-D-glucuronide (X-Gluc), 3-amino-9-ethyl carbazol (AEC), fuchsin, iodonitrotetrazolium (INT), tetrazolium blue and tetrazolium violet. Alternatively, an enzyme can be used in a metallographic detection scheme. Metallographic detection methods include using an enzyme such as alkaline phosphatase in combination with a water-soluble metal ion and a redox-inactive substrate of the enzyme. The substrate is converted to a redox-active agent by the enzyme, and the redox-active agent reduces the metal ion, causing it to form a detectable precipitate. (See, for example, co-pending U.S. patent application Ser. No. 11/015,646, filed Dec. 20, 2004, PCT Publication No. 2005/003777 and U.S. Patent Application Publication No. 2004/0265922; each of which is incorporated by reference herein). Metallographic detection methods include using an oxido-reductase enzyme (such as horseradish peroxidase) along with a water soluble metal ion, an oxidizing agent and a reducing agent, again to form a detectable precipitate. (See, for example, U.S. Pat. No. 6,670,113, which is incorporated by reference herein). Haptens are small molecules that are specifically bound by antibodies, although by themselves they will not elicit an immune response in an animal and must first be attached to a larger carrier molecule such as a protein to generate an immune response. Examples of haptens include di-nitrophenyl, biotin, digoxigenin, and fluorescein. Additional examples of oxazole, pyrazole, thiazole, nitroaryl, benzofuran, triperpene, urea, thiourea, rotenoid, coumarin and cyclolignan haptens are disclosed in U.S. Pat. No. 7,985,557, issued May 21, 2013, which is incorporated by reference herein. This is not an exhaustive review of all possible labeling schemes, and other useful labels and labelling schemes may be currently available or developed in the future.

In a specific embodiment, the biomarker-specific reagent is an antibody (termed “primary antibody”) and the detection reagents include an antibody capable of binding to the primary antibody (termed “secondary antibody”) and a detectable entity including an enzyme coupled to or adapted to be coupled to the secondary antibody and reagents reactive with the enzyme to deposit a chromogen or fluorophore on the sample. In an embodiment, the secondary antibody has affinity for immunoglobulins from a specific animal species from which the primary antibody is derived (termed a “species-specific secondary antibody”). In another embodiment, the secondary antibody is reactive with a tag incorporated into the primary antibody, such as an epitope tag located in the primary amino acid sequence of the primary antibody or a hapten coupled to a reactive side chain of the primary antibody.

In some embodiments, the biomarker-specific reagents are applied using a multiplex method. For example, where the biomarker is a protein, multiple primary antibodies may be applied to a single serial section. The primary antibodies must be applied in a manner that allows the different biomarkers to be differentially labeled.

One way to accomplish differential labelling of different biomarkers is to select primary antibody/secondary antibody/enzyme combinations that will not result in off-target cross-reactivity between different antibodies or detection reagents (termed “combination staining”). For example, each secondary antibody used may bind to only one of the primary antibodies used on the serial section. For example, primary antibodies could be selected that are derived from different animal species (such as mouse, rabbit, rat, and got antibodies), in which case species-specific secondary antibodies may be used. As another example, each primary antibody may include a different hapten or epitope tag, and the secondary antibodies are selected to specifically bind to the hapten or epitope tag. Additionally, each secondary antibody should be adapted to deposit a different detectable entity on the serial section, such as by linking a different enzyme to each secondary antibody. An example of such an arrangement is shown at U.S. Pat. No. 8,603,765. Such arrangements have the potential advantage of being able to have each primary:secondary antibody pair present on the sample at the same time and/or to perform staining with primary and/or secondary antibody cocktails, which reduced the number of staining steps. However, such arrangements may not always be feasible, as reagents may cross-react with different enzymes, and the various antibodies may cross-react with one another, leading to aberrant staining.

Another way to accomplish differential labelling of different biomarkers is to sequentially stain the sample for each biomarker. In such an embodiment, a first primary antibody is reacted with the serial section, followed by a secondary antibody binding to the primary antibody and other detection reagents resulting in deposition of a first detectable entity. The serial section is then treated to remove the primary and secondary antibodies and the process is repeated for subsequent primary antibodies. Examples of methods for removing the primary and secondary antibodies include heating the sample in the presence of a buffer that elutes the antibodies from the sample (termed a “heat-kill method”), such as those disclosed by Stack et al., Multiplexed immunohistochemistry, imaging, and quantitation: A review, with an assessment of Tyramide signal amplification, multispectral imaging and multiplex analysis, Methods, Vol. 70, Issue 1, pp 46-58 (November 2014), and PCT/EP2016/057955, the contents of which are incorporated by reference. As will be appreciated by the skilled artisan, combination staining and sequential staining methods may be combined. For example, where only a subset of the primary antibodies are compatible with combination staining, the sequential staining method can be modified, wherein the antibodies compatible with combination staining are applied to the sample using a combination staining method, and the remaining antibodies are applied using a sequential staining method. Many other alternatives will be known to the skilled artisan, and the present methods should not be construed to be limited to any particular staining method.

The detection and (where performed) quantification of protein biomarkers can be performed manually (for example, microscopically by a trained pathologist) or automatically. Where automated detection and quantification is performed, a brightfield or fluorescence detection system and/or slide scanner may be used to capture digital images of the stained samples, and then using automated image analysis systems to detect cells having the phenotypes described herein, to quantitate the various cell populations, and/or to calculate likelihoods of progression or response to specific treatment courses based on the cell counts. To this end, biological image analysis devices are further provided, which function to capture and/or to analyze the image of the sample according to the presently disclosed methods. The biological image analysis device includes at least a processor and a memory coupled to the processor, the memory to store computer-executable instructions that, when executed by the processor, cause the processor to perform operations.

The term “processor” encompasses all kinds of apparatus, devices, and machines for processing data, including by way of example a programmable microprocessor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., an LCD (liquid crystal display), LED (light emitting diode) display, or OLED (organic light emitting diode) display, for displaying information to the user and a keyboard and a pointing device, e.g., a mouse or a trackball, by which the user can provide input to the computer. In some implementations, a touch screen can be used to display information and receive input from a user. Other kinds of devices can be used to provide for interaction with a user as well; for example, feedback provided to the user can be in any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include any number of clients and servers. A client and server are generally remote from each other and typically interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

The skilled artisan will appreciate that the biological image analysis device described herein may be included within systems comprising additional components, e.g. analyzers, scanners, etc. For example, the biological image analyzer may be communicatively coupled to a computer-readable storage medium containing a digital copy of the image of the biological sample. Alternatively, the biological image analysis device may be communicatively coupled to an imaging apparatus. In general, an imaging apparatus can include, without limitation, one or more image capture devices. Image capture devices can include, without limitation, a camera (e.g., an analog camera, a digital camera, etc.), optics (e.g., one or more lenses, sensor focus lens groups, microscope objectives, etc.), imaging sensors (e.g., a charge-coupled device (CCD), a complimentary metal-oxide semiconductor (CMOS) image sensor, or the like), photographic film, or the like. In digital embodiments, the image capture device can include a plurality of lenses that cooperate to prove on-the-fly focusing. A CCD sensor can capture a digital image of the specimen. One method of producing a digital image includes determining a scan area comprising a region of the microscope slide that includes at least a portion of the specimen. The scan area may be divided into a plurality of “snapshots.” An image can be produced by combining the individual “snapshots.” In some embodiments, the imaging apparatus produces a high-resolution image of the entire specimen, one example for such an apparatus being the VENTANA iScan HT slide scanner from Ventana Medical Systems, Inc. (Tucson, Ariz.). The system can also include a desktop computer, a laptop computer, a tablet, or the like and can include digital electronic circuitry, firmware, hardware, memory, a computer storage medium, a computer program, a processor, or the like. The images can also be divided into a matrix of pixels. The pixels can include a digital value of one or more bits, defined by the bit depth. A network or a direct connection may interconnect the imaging apparatus and the computer system. The computer systems include one or more processors that are programmed with a series of computer-executable instructions, the instructions being stored in a memory.

When executed, instructions (which may be stored in the memory) cause at least one of the processors of the computer system to receive an input, which is a color image comprising a biological sample. Once the necessary inputs are provided, a module is then executed to perform the various functions of the methods described herein.

In an aspect, the biological image analysis device is capable of performing manual or automated cell counts with manual or automated deconvolution of color or color—enhanced images. A number of methods for deconvolution are known, including, for example, those disclosed by WO 2016/016306 A1; Chen & Srinivas, Group sparsity model for stain unmixing in brightfield multiplex immunohistochemistry images, Comput Med Imaging Graph., 46 Pt 1:30-9 (December 2015); Ruifrok & Johnston, Quantification of Histochemical Staining by Color Deconvolution, Anal. Quant. Cytol. Histol. 23, 291-299 (2001), Rabinovich, et al., Unsupervised Color Decomposition of Histologically Stained Tissue Samples, Advances in Neural Information Processing Systems 16 (NIPS 2003). Cells having stain colors correlating to the identified phenotypes are counted and logged.

The stained samples are then scored for expression of the protein being detected. Common IHC scoring methods include: percent positivity (i.e. the number of cells expressing the biomarker above a threshold level), staining intensity (i.e. a score of 1 for weak intensity staining; a score of 2 for medium intensity staining, or a score of 3 for strong intensity staining), weighted percent positivity scores (i.e. calculating the percentage of cells at each of a plurality of intensity levels, such as is done with an H-score). For MACC1 expression, any scoring method that maintains statistical significance between “low” and “high” cohorts may be used. In one specific example, histochemical images stained for MACC1 protein are scored on the basis of percent positivity (i.e., the percentage of cells having unequivocal MACC1 protein expression).

### VI. Nucleic Acid Amplification and Detection

A nucleic acid sample can be used for detection and quantification, e.g., using nucleic acid amplification, e.g., using any primer-dependent method. In some embodiments, a preliminary reverse transcription step is carried out (also referred to as RT-PCR, not to be confused with real time PCR). See, e.g., Hierro et al. (2006) 72:7148. The term “qRT-PCR” as used herein refers to reverse transcription and quantitative PCR. Both reactions can be carried out in a single tube without interruption, e.g., to add reagents. For example, a polyT primer can be used to reverse transcribe all mRNAs in a sample with a polyA tail, random oligonucleotides can be used, or a primer can be designed that is specific for a particular target transcript that will be reverse transcribed into cDNA. The cDNA, or DNA from the sample, can form the initial template to be for quantitative amplification (real time or quantitative PCR, i.e., RT-PCR or qPCR). qPCR allows for reliable detection and measurement of products generated during each cycle of PCR process. Such techniques are well known in the art, and kits and reagents are commercially available, e.g., from Roche Molecular Systems, Life Technologies, Bio-Rad, etc. See, e.g., Pfaffl (2010) Methods: The ongoing evolution of qPCR vol. 50.

A separate reverse transcriptase and thermostable DNA polymerase can be used, e.g., in a two-step (reverse transcription followed by addition of DNA polymerase and amplification) or combined reaction (with both enzymes added at once). In some embodiments, the target nucleic acid is amplified with a thermostable polymerase with both reverse transcriptase activity and DNA template-dependent activity. Exemplary enzymes include Tth DNA polymerase, the C. therm Polymerase system, and those disclosed in US20140170730 and US20140051126.

Probes for use as described herein can be labeled with a fluorophore and quencher (e.g., TaqMan, LightCycler, Molecular Beacon, Scorpion, or Dual Labeled probes). Appropriate fluorophores include FAM, JOE, TET, Cal Fluor Gold 540, HEX, VIC, Cal Fluor Orang 560, TAMRA, Cyanine 3, Quasar 570, Cal Fluor Red 590, Rox, Texas Red, Cyanine 5, Quasar 670, and Cyanine 5.5. Appropriate quenchers include TAMRA (for FAM, JOE, and TET), DABCYL, and BHQ1-3. In other embodiments, amplicon generation can be tracked and quantified using a double-stranded nucleic acid intercalating dye, such as SYBR Green, SYBR Gold, ethidium bromide, YO-PRO-1, LC GREEN, SYTO9, SYT082, SYTO13, EVAGREEN, CHROMOFY, BOXTO, and BEBO dyes. An overview of different chemistries useful in qPCR is presented by Navarro et al., Clin Chim Acta. Vol. 439, pp. 231-50 (2015 Jan. 15), the content of which is incorporated by reference in its entirety.

Detection devices are known in the art and can be selected as appropriate for the selected labels. Detection devices appropriate for quantitative PCR include the Cobas® and Light Cycler® systems (Roche), PRISM 7000 and 7300 real-time PCR systems (Applied Biosystems), etc. Six-channel detection is available on the CFX96 Real Time PCR Detection System (Bio-Rad) and Rotorgene Q (Qiagen), allowing for a higher degree of multiplexing.

Results can be expressed in terms of a threshold cycle (abbreviated as Ct, and in some instances Cq or Cp). A lower Ct value reflects the rapid achievement of a predetermined threshold level, e.g., because of higher target nucleic acid concentration or a more efficient amplification. A higher Ct value may reflect lower target nucleic acid concentration, or inefficient or inhibited amplification. The threshold cycle is generally selected to be in the linear range of amplification for a given target. In some embodiments, the Ct is set as the cycle at which the growth signal exceeds a pre-defined threshold line, e.g., in relation to the baseline, or by determining the maximum of the second derivation of the growth curve. Determination of Ct is known in the art, and described, e.g., in U.S. Pat. No. 7,363,168.

### VII. Selection of Cutpoint Between High MACC1 Expression and Low MACC1 Expression

The present assays are based in part on determination of “high” versus “low” expression of a MACC1 gene product.

Any cutoff that maintains a statistically significant difference between MACC1-high/pMMR, and MACC1-low/pMMR stage II colorectal cancer patients in Kaplan-Meier curves may be used. For example, in the histochemical examples below, a 75% cutoff was used, although others may be used as well. For example, a quartile cutoff may be selected, such as 25%, 50%, or 75%. As another example, a quintile cutoff may be selected, such as 20%, 40%, 60%, 80%, or 100%. In another example, a decile cutoff may be selected, such as 10%, 20%, 30%, 40%, 50%, 60%, 70%, 80%, or 90%. In another example, a cutoff may be selected from 5%, 10%, 15%, 20%, 25%, 30%, 35%, 40%, 45%, 50%, 55%, 60%, 65%, 70%, 75%, 80%, 85%, 90%, or 95%. In another embodiment, the cutoff value may be selected statistically. For example, Zlobec et al. (incorporated herein by reference), disclose a method of selecting cutoffs for immunohistochemical assays ROC curve analysis. Such a method could be used for either histochemical or qRT-PCR analyses.

### VIII. Colon and Colorectal Cancer and Treatments

In some embodiments, a chemotherapy or chemotherapeutic is administered to patients having pMMR status and high MACC1 expression level. The chemotherapy typically comprises at least a fluoropyrimidine-based chemotherapy or the chemotherapeutic typically comprises at least fluoropyrimidine. Examples of fluoropyrimidine-based chemotherapy or chemotherapeutics include capecitabine, floxuridine, and fluorouracil (5-FU). The fluoropyrimidine-based chemotherapy or the fluoropyrimidine chemotherapeutic may be used alone or as combination therapies, including but not limited to combinations with other fluoropyrimidine-based chemotherapies or fluoropyrimidine chemotherapeutics; non-fluoropyrimidine-based chemotherapies or non-fluoropyrimidine chemotherapeutics, such as alkylating agents (e.g. oxaliplatin), cytotoxic chemotherapies (such as irinotecan); targeted therapies or targeted chemotherapeutics (e.g., VEGF-targeted therapies or VEGF-targeted chemotherapeutics including bevacizumab (AVASTIN), ziv-aflibercept (ZALTRAP), or ramucirumab (CYRAMZA); EGFR-targeted therapies including cetuximab (ERBITUX) or panitumumab (VECTIBIX); and multi-kinase inhibitors such as regorafenib (STIVARGA)); immunotherapies or immunotherapeutics, including checkpoint inhibitor-directed therapies (such as PD-1 or PD-L1 directed monoclonal antibodies), adoptive cell transfers (such as T-cells engineered to express chimeric antigen receptors (CAR-T)), oncolytic virus therapy or oncolytic virus therapeutics, therapeutic vaccines (e.g. recombinant peptides corresponding to tumor-specific HLA ligands), adjuvant immunotherapy or adjuvant immunotherapeutics, and cytokines; radiation therapy; folic acid derivatives (such as leucovorin); and chemoprotectants. The therapies or therapeutics may be administered as adjuvant therapy or adjuvant therapeutics and/or neoadjuvant therapy or neoadjuvant therapeutics or, where appropriate, as a front line treatment (i.e. when the patient is not well enough for surgery).

As used herein, the term “non-chemotherapeutic” or “treatment does not comprise a chemotherapy” or similar expression comprises immunotherapeutics, including checkpoint inhibitors (such as PD-1 or PD-L1 directed monoclonal antibodies), cells for adoptive cell transfers (such as T-cells engineered to express chimeric antigen receptors (CAR-T)), oncolytic virus therapeutics, therapeutic vaccines (e.g. recombinant peptides corresponding to tumor-specific HLA ligands), adjuvant immunotherapeutics, and cytokines; radiation therapy; folic acid derivatives (such as leucovorin); and chemoprotectants. The therapeutics or therapies may be administered as adjuvant therapy or adjuvant therapeutics and/or neoadjuvant therapy or neoadjuvant therapeutics or, where appropriate, as a front line treatment (i.e. when the patient is not well enough for surgery).

In an embodiment, the treatment course selected is based on the stage of the cancer.

Stage 0 colorectal cancers are cancers that have not grown beyond the inner lining of the colon. Stage I colorectal cancers are cancers that have not spread outside of the colon wall itself or into nearby lymph nodes. Stage 0 and Stage I cancers are typically treated with only surgery, although if the physician so desires, a fluoropyrimidine-based chemotherapy may be administered based on the MMR status and MACC1 expression level according to the methods described herein.

Stage II colorectal cancers are cancers that have grown through the wall of the colon, and possibly into nearby tissue, but have not yet spread to the lymph nodes. Surgical removal of the tumor and nearby lymph nodes is typically performed at this stage, and may be accompanied by adjuvant chemotherapy and/or radiation therapy. Common chemotherapies include fluoropyrimidine-based chemotherapies, optionally in combination with leucovorin. Radiation therapy may also be appropriate at this stage. In one specific non-limiting embodiment, a method of treating a stage II colorectal cancer may comprise:
for subjects having a pMMR status and high MACC1 expression level, administering an adjuvant therapy comprising a fluoropyrimidine-based chemotherapy, optionally in combination with leucovorin and optionally in combination with radiation therapy; or
for subjects having a dMMR status or a pMMR status with low MACC1 expression level, administering a therapy course comprising:
   post-surgical monitoring, optionally in combination with adjuvant radiation therapy; or
   chemotherapy that does not comprise a fluoropyrimidine-based chemotherapy, optionally in combination with radiation therapy, either without surgery or as an adjuvant therapy.

Stage III colorectal cancers are cancers that have spread to nearby lymph nodes, but have not yet spread to other parts of the body. Surgical removal of the tumor or a partial colectomy (including removal of nearby lymph nodes) followed by adjuvant chemotherapy and/or radiation therapy is typically performed at this stage, although the chemotherapy (optionally in combination with radiation therapy) may be used without surgery for certain patients. Common chemotherapies include fluoropyrimidine-based chemotherapies, optionally in combination with leucovorin and/or alkylating agents (such as oxaliplatin). Non-limiting combination therapies used at this stage include FOLFOX (5-FU, leucovorin, and oxaliplatin) or CapeOx (capecitabine and oxaliplatin). In one specific non-limiting embodiment, a method of treating a stage III colorectal cancer may comprise:
for subjects having a pMMR status and high MACC1 expression level, administering a fluoropyrimidine-based chemotherapy, optionally in combination with leucovorin, optionally in combination with radiation therapy, and optionally in combination with an alkylating chemotherapy (such as oxaliplatin), either without surgery or as an adjuvant therapy; or
for subjects having a dMMR status or a pMMR status with low MACC1 expression level, administering a therapy course comprising a chemotherapy that does not comprise a fluoropyrimidine-based chemotherapy and/or a radiation therapy, either without surgery or as an adjuvant therapy.

Stage IV colorectal cancers are cancers that have spread from the colon to distant organs and tissues. Surgical removal of the tumor or a partial colectomy (including removal of nearby lymph nodes) and metastases (if possible), as well as chemotherapy and/or radiation therapy is typically performed at this stage. Common chemotherapies include fluoropyrimidine-based chemotherapies, optionally in combination with leucovorin and/or other chemotherapies and/or targeted therapies. Non-limiting combination therapies used at this stage include:
FOLFOX: leucovorin, 5-FU, and oxaliplatin (ELOXATIN);
FOLFIRI: leucovorin, 5-FU, and irinotecan (CAMPTOSAR);
CapeOX: capecitabine (XELODA) and oxaliplatin;
FOLFOXIRI: leucovorin, 5-FU, oxaliplatin, and irinotecan;
One of the above combinations plus either a drug that targets VEGF (such as bevacizumab [AVASTIN], ziv-aflibercept [ZALTRAP], or ramucirumab [CYRAMZA]), or a drug that targets EGFR (such as cetuximab [Erbitux] or panitumumab [VECTIBIX]);
5-FU and leucovorin, with or without a targeted drug;
Capecitabine, with or without a targeted drug;
Irinotecan, with or without a targeted drug;
Cetuximab alone;
Panitumumab alone;
Regorafenib (STIVARGA) alone; and
Trifluridine and tipiracil (LONSURF),
   In one specific non-limiting embodiment, a method of treating a stage IV colorectal cancer may comprise:
for subjects having a pMMR status and high MACC1 expression level, administering a fluoropyrimidine-based chemotherapy, optionally in combination with a folic acid derivative, a chemoprotectant, a radiation therapy, a non-fluoropyrimidine-based chemotherapy, a targeted therapy, and/or an immunotherapy, either without surgery, as a neoadjuvant therapy, or as an adjuvant therapy; or
for subjects having a dMMR status or a pMMR status with low MACC1 expression level, administering a therapy course comprising: a chemotherapy that does not comprise a fluoropyrimidine-based chemotherapy, a radiation therapy, a targeted therapy, and/or an immunotherapy, the therapy course administered without surgery, as a neoadjuvant therapy, or as an adjuvant therapy.

### VIII. Kits

In an embodiment, kits are provided for performing the methods disclosed herein. The kits generally will comprise at least one MACC1 gene product-biomarker specific agent and instructions for use with a pMMR colorectal sample. In some embodiments, the kits may further comprise a set of biomarker-specific agents for determining MMR status of the sample. In an embodiment, the set of biomarker specific agents includes an MLH1 gene product-biomarker-specific agent, an MSH2 gene product-biomarker-specific agent, an MSH6 gene product-biomarker-specific agent, and a PMS2 gene product-biomarker-specific agent. The biomarker specific agents may be specific for nucleic acid gene products or proteinaceous gene products as desired.

In embodiments in which the kits are to be used in histochemical or cytochemical detection of at least one biomarker, the kits may further comprise additional components for such detection processes. For example, detection reagents for depositing chromogenic, fluorescent, or other stains compatible with histochemical or cytochemical detection of biomarkers may be provided. Such kits may further include counterstains, such as hematoxylin, Azure B, Giemsa stain, Nuclear fast red (Kernechtrot), Methyl green, Hoechst stain, 4′, 6-diamidino-2-phenylindole (DAPI), Propidium iodide, and Fluorophore-tagged phalloidin, among others. These kits may further include buffers and other reagents necessary to perform RNA-in situ hybridization for mRNA biomarkers or histochemical or cytological detection of proteinaceous biomarkers. In some embodiments, the kit components are formulated for use on an automated slide stainer, such as a VENTANA BenchMark series IHC/ISH stainer, a Leica BOND series IHC/ISH stainer, a Dako AUTOSTAINER series IHC/ISH stainer, or the like.

In an embodiment, a kit is provided wherein the MACC1 gene product-biomarker specific agent is a MACC1-specific primer set useful in amplification-based detection of MACC1 mRNA. In an embodiment, a MACC1-specific primer set is provided comprising: (1) a forward primer comprising, consisting essentially of, or consisting of one or more of SEQ ID NO: 1 and SEQ ID NO: 5; (2) a reverse primer comprising, consisting essentially of, or consisting of one or more of SEQ ID NO: 2 and SEQ ID NO: 6; (3) a forward primer comprising, consisting essentially of, or consisting of SEQ ID NO: 1 and a reverse primer comprising, consisting essentially of, or consisting of SEQ ID NO: 2; or (4) a forward primer comprising, consisting essentially of, or consisting of SEQ ID NO: 5 and a reverse primer comprising, consisting essentially of, or consisting of SEQ ID NO: 6. In another exemplary embodiment, the kit comprises a primer pair and a detection probe in a combination as set forth in Table 1. In embodiments in which the kits are to be used in amplification-based detection of at least one biomarker, the kits may further comprise additional components for such detection processes. For example, the kits may include buffers, dNTPs, and other elements (e.g., cofactors or aptamers) appropriate for reverse transcription and amplification. Typically, the mixture is concentrated, so that an aliquot is added to the final reaction volume, along with sample (e.g., DNA), enzymes, and/or water. In some embodiments, the kit further comprises reverse transcriptase (or an enzyme with reverse transcriptase activity), and/or DNA polymerase (e.g., thermostable DNA polymerase such as Taq, ZO5, and derivatives thereof). In some embodiments, the kit further includes components for DNA or RNA purification from a sample, e.g., a non-invasive or tissue sample. For example, the kit can include components from MagNA Pure LC Total Nucleic Acid Isolation Kit, DNA Isolation Kit for Cells and Tissues, DNA Isolation Kit for Mammalian Blood, High Pure FFPET DNA Isolation Kit, High Pure or MagNA Pure RNA Isolation Kits (Roche), DNeasy or RNeasy Kits (Qiagen), PureLink DNA or RNA Isolation Kits (Thermo Fisher), etc.

### IX. Examples

### A. Materials and Methods

A1. Patient Populations

A1a. Discovery and Comparison Cohorts: Charité 1 and 2

Primary tumor samples were obtained from patients who gave informed written consent. The protocol was approved by the local ethics committee of the Charité Universitätsmedizin, Berlin, Germany. For inclusion, patients had to have: received no preoperative treatment for their cancer; no history of familial colorectal cancer; no second tumor of the same or a different entity; undergone RO resection; and tumors that were staged and typed according to Union for International Cancer Control and World Health Organization guidelines.

Following surgery, primary tumor tissues were either immediately shock frozen in liquid nitrogen or fixed as formalin-fixed paraffin-embedded (FFPE) tissue according to standard protocols. The histopathology of each sample used for subsequent experimental analysis was reviewed by an experienced pathologist to confirm diagnosis, tissue composition, and tumor content. Microdissection of the tumor cell populations for subsequent RNA isolation was performed on all cryo-preserved and FFPE samples.

A1b. Test and Validation Cohorts: BIOGRID 1 and 2

Patients were identified from the prospective Australian Comprehensive Cancer Outcomes and Research Database (ACCORD) colorectal cancer database. ACCORD is maintained and managed by BIOGRID Australia® and includes prospectively collected multidisciplinary data relating to diagnosis, histopathological features, patient characteristics, treatment, and outcomes for all patients treated at participating sites. Point of care follow-up data are collected at each clinical visit, including any cancer recurrence. Eligibility criteria for the current study included surgical resection of stage II colon cancer at the Royal Melbourne Hospital or Western Hospital, Melbourne, Australia, between 2001 and 2011, available archived tumor whole tissue sections, and follow-up data for at least 24 months. To avoid the doubts cast by inadequate statistical power, the BIOGRID 1 test cohort was purposefully enriched for patients who had disease recurrence to increase the overall recurrence rate to 25%. The BIOGRID 2 validation cohort included consecutive patients with stage II colon cancer and was not enriched for recurrence.

A2. Assays

A2a. DNA and RNA Isolation: Charité 1 and 2, and BIOGRID 1 Cohorts

All DNA and RNA isolations of cryo-preserved and FFPE materials from Charité 1 and 2 patients used microdissected tumor cell populations after evaluation by a trained pathologist. Genomic DNA was isolated from cryo-preserved tumor tissues of Charité 1 patients using the Charge Switch gDNA Micro Tissue Kit (Invitrogen) according to the manufacturer's protocol (Ilm et al., Mol Cancer 2015). Total RNA was isolated from cryo-preserved tumor tissues from Charité 1 and 2 patients using TRIzol reagent (Invitrogen), including a DNase step according to the manufacturer's protocol.

Total RNA was isolated from FFPE samples of Charité 2 and BIOGRID 1 patients using four sections per tumor, each 4 μm thick. Paraffin was removed by incubation in xylene and absolute ethanol, for 5 min each, followed by air drying. RNA isolation was performed employing the High Pure FFPET RNA Isolation Kit (Roche Diagnostics GmbH), according to the manufacturer's protocol. Eluted DNA and RNA were quantified using the Nanodrop 1000 (Peqlab Biotechnologie). RNA quality was proven using the Agilent 2100 Bioanalyzer (Agilent).

A2b. PCR-Based Microsatellite Instability Analysis: Charité 1 Cohort

DNA from the Charité 1 cohort was analyzed using the MSI Analysis System (Promega, Madison, Wis., USA) according to the manufacturer's protocol, including five mononucleotide repeat primers s (NR-21, BAT-26, BAT-25, NR-24, MONO-27; for genes SLC7A8, MSH2, c-kit, ZNF-2, and MAP4K3, respectively). These mononucleotides are the most sensitive and specific markers for detection of microsatellite instability (MSI)-high (MSI-H) tumors. Two additional pentanucleotide markers were included for identification of sample cross-contamination (Penta C and Penta D) (Bacher et al. Dis Markers 2004). Polymerase chain reaction (PCR) products were sequenced on an ABI 3700 sequencer (Applied Biosystems). Tumors were classified as microsatellite stable (MSS)/MSI-low (MSI-L) (0 or 1 markers demonstrating instability) or MSI-H (≥2 markers demonstrating instability), as previously described (Ilm et al. Mol Cancer 2015).

A2c. Immunohistochemistry-Based Mismatch Repair Analysis: BIOGRID 1 and 2 Cohorts

All immunohistochemistry assays were developed and performed on the VENTANA BenchMark XT automated staining instrument at Ventana Medical Systems, Inc. (Tucson, Ariz., USA). Mismatch repair (MMR) status was assessed using anti-MLH1 (M1), MSH2 (G219-1129), CONFIRM anti-MSH6 mouse monoclonal, and PMS2 (EPR3947) rabbit monoclonal primary antibodies (Ventana Medical Systems, Inc.).

FFPE samples (4 μm sections) were deparaffinized, pretreated with Cell Conditioning 1 for antigen retrieval (64 min for MLH1, PMS2, and MSH6; 40 min for MSH2), treated to inactivate the endogenous peroxidases, and then incubated with anti-MLH1 primary antibody at room temperature, and with PMS2, MSH2, and MSH6 primary antibodies at 37° C. for 12 min. Antigen-antibody reactions were visualized using OptiView DAB Detection Kit (Ventana Medical Systems, Inc.). To enhance the DAB signal of PMS2 detection, signal amplification (OptiView Amplification Kit, Ventana Medical Systems, Inc.) was utilized for 8 min. After chromogenic detection, all slides were counterstained with hematoxylin II and bluing reagent (Ventana Medical Systems, Inc.) for 4 min each.

Immunostaining of MMR markers was evaluated for the presence or absence of nuclear protein expression within tumor cells in the presence of nuclear staining within the internal control cells (lymphocytes, stromal cells, or normal colonic epithelium). The sample was considered MMR deficient (dMMR) if the tumor cells lacked staining for one or more MMR proteins and MMR proficient (pMMR) if all four MMR proteins were present in malignant cells.

A2d. Quantitative RT-PCR for MACC1 Levels: Charité 1 and 2, and BIOGRID 1 Cohorts

MACC1 mRNA expression levels in samples from the Charité 1 and 2 cohorts were determined with the following two-step quantitative real-time reverse transcriptase-PCR: The reverse transcriptase reaction was performed with 50 ng of total RNA. Quantitative real-time PCR for MACC1 was performed in duplicate in a total volume of 10 μL (95° C. for 60 s, 45 cycles of 95° C. for 10 s, 60° C. for 10 s, 72° C. for 20 s) using the LightCycler® 480 (DNA Master Hybridization Probes kit, Roche Diagnostics GmbH) and the primers and probes (synthesized by BioTeZ and TIB MolBiol, Berlin, Germany), as previously described (Stein et al., Nat Med 2009).

MACC1 mRNA expression levels in the BIOGRID 1 samples were determined with the following one-step duplex quantitative real-time reverse transcriptase-PCR: Reactions were performed in triplicate in a total volume of 20 μL using the TaqMan® RNA Amplification Kit (internal Roche product) containing a final concentration of 500 nM of each primer, 100 nM of each probe and 4 μL of total RNA (at least 10 ng). Glucose-6-phosphate-dehydrogenase (G6PD) and hypoxanthine phosphoribosyltransferase1 (HPRT) were employed as housekeeping genes. All reactions were performed on a LightCycler® 480 with the following cycling parameters: 50° C. for 5 min/95° C. for 1 min/61° C. for 30 min (UNG/RT), 2 cycles of 95° C. for 15 s/61° C. for 30 s (amplification 1), and 53 cycles of 92° C. for 15 s/61° C. for 30 s (amplification 2) followed by a final stage of 40° C. for 30 s (cooling). The results were analyzed using the relative quantification tool of the LightCycler® 480 software (version 1.5; Roche Diagnostics GmbH). All primers and probes are shown in Table 1. Abbreviations in Table 1 are as follows: BHQ, black hole quencher; G6PD, glucose-6-phosphate-dehydrogenase gene; HPRT, hypoxanthine phosphoribosyltransferase1 gene; FAM, 6-carboxyfluorescein; FITC, fluorescein isothiocyanate; MACC1, Metastasis Associated in Colon Cancer 1 gene; PCR, polymerase chain reaction.

A2e. Immunohistochemistry for MACC1 Protein Expression: BIOGRID 1 and 2 Cohorts

Immunohistochemistry for MACC1 protein expression was analyzed in FFPE full tissue sections (4 μm) on a BenchMark XT automated slide stainer (Ventana). Briefly, the specimens were deparaffinized, pretreated with Cell Conditioning 1 for 64 min to retrieve the epitopes, followed by inactivation of endogenous peroxidases. Sections were incubated with anti-MACC1 rabbit polyclonal antibody (1:75, Sigma HPA020103) at 37° C. for 16 min. The presence of MACC1 protein was visualized using OptiView DAB Detection Kit. Following chromogenic detection, all slides were counterstained with hematoxylin II and bluing reagent for 4 min each.

MACC1 staining was scored both for staining intensity and percent staining by a pathologist who remained blinded to the patients' clinical outcome. The scoring algorithm was developed in the BIOGRID 1 test cohort by evaluating several algorithms that incorporated both staining intensity and percent staining. The final scoring algorithm only included percent staining of viable tumor cells. The sample was considered to be MACC1 positive when >75% of tumor cells demonstrated unequivocal cytoplasmic staining.

A3. Statistical Analysis

Sample size was not statistically derived and was based on case/cohort availability.

No estimate of effect size was available prior to this study, as this study was a first attempt to determine if a potentially relevant clinical association with MACC1 expression existed. For this reason, caution was taken not to rely on p-values alone a indicative of potential utility. For our purposes, Kapan-Meier plots and associated log-rank statistics were used to assess potential utility and a significance level of 0.10 indicated marginal evidence of a potentially clinically relevant finding, a significance level of 0.05 indicated strong evidence of a potentially cinically relevant finding, and consistency of effect size across cohorts indicated strong evidence of a potentially clinically relevant finding. After assessment of stratification in each cohort, cohorts B1 and B2 were pooled to obtain the most precise estimate of MACC1 informatively.

In order to determine appropriate cutpoints for stratification based on MACC1 expression in the B1 training cohort, a single-step ROC based method was used for rtPCR and a multi-step method was used for IHC. These strata definitions were then applied to the B2 cohort to assess validity. For rtPCR, ROC analysis was used to find the optimum cutpoint based on maximizing the sum of sensitivity and specificity, which is an analog of the Youden's Index. For MC, we first determined an ROC-based optimum cutpoint maximizing the sum of sensitivity and specificity, then assembled scientific and pathology subject matter experts to assess positive, negative, and borderline slides to agree on a repeatable and reproducible cutpoint in the neighborhood of the statistically derived cutpoint. This was deemed an important step to ensure that future use of MACC1 expression would be best adapted to clinical practice. The statistically derived optimum was 80% positive staining, which was adjusted down to 75% based on scientist and pathologist input, and did not change the status of any cases in our training cohort.

For the Charité 1 cohort and BIOGRID 1 and 2 cohorts, all statistical analyses and Kaplan-Meier plots were performed using the R programming environment (REFS) or SAS Software (SAS System for Windows Version 9.4; SAS Institute Inc., Cary, N.C., USA), where a significance level of 0.05 indicated a potentially clinically relevant finding. Recurrence-free survival (RFS) was defined for all cohorts as the time from the date of surgery to the date of disease relapse, or was censored at the date of the last follow-up visit for relapse-free patients.

For the cohort Charité 2, all statistical analyses were performed with IBM® SPSS® Statistics (Version 21; IBM Corp., Armonk, N.Y., USA). Receiver operating characteristic (ROC) analysis was performed on MACC1 mRNA expression levels to distinguish low and high MACC1 expression groups. ROC curves and statistical analysis is shown at FIG. 8. Clinical follow-up information was obtained from the tumor bank of the Charité Comprehensive Cancer Center (Berlin) after completing mRNA quantification and MSI status. The Kaplan-Meier method was used to estimate cumulative survival rates and significance of differences in survival rates were assessed using the log-rank test. Metastasis-free survival (MFS) time was defined as the time period from the date of surgery to the date of confirmed distant metastases or to the date of last follow-up contact/death for censored patients.

TABLE 1
PCR primers and probes for MACC1, G6PD, and HPRT

| Appli- | | |
|---|---|---|
| cation | Primer/Probe | Sequence 5′ → 3′ |
| MACC1 | Primer MACC1 | TTC TTT TGA TTC CTC CGG TGA |
| PCR | forward | (SEQ ID NO: 1) |
| | Primer MACC1 | ACT CTG ATG GGC ATG TGC TG |
| | reverse | (SEQ ID NO: 2) |
| | Hybridi- | GCA GAC TTC CTC AAG AAA TTC |
| | zation FITC- | TGG AAG ATC TA (SEQ ID NO: 3) |
| | labeled | |
| | MACC1 probe | |
| | Hybridi- | AGT GTT TCA GAA CTT CTG GAC |
| | zation LCRed | ATT TTA GAC GA (SEQ ID NO: 4) |
| | 640-labeled | |
| | MACC1 probe | |
| MACC1 | Primer MACC1 | ATT GAC ATG GAA GCT GGA AAA |
| duplex | forward | CTC (SEQ ID NO: 5) |
| PCR | Primer MACC1 | CAC GAA GGG TGA AAG CAT CC |
| MACC1/ | reverse | (SEQ ID NO: 6) |
| G6PD | Hydrolysis | TAC AGA ATG CCA GGA CCC AGA |
| MACC1/ | FAM/BHQ2- | CTT GCT TCA CAA TTG G (SEQ |
| HPRT | labeled | ID NO: 7) |
| | MACC1 probe | |
| G6PD | Primer G6PD | TGC TGT GTC TGG TGG CC |
| duplex | forward | (SEQ ID NO: 8) |
| PCR | Primer G6PD | GCA TTT CAA CAC CTT GAC CTT |
| MACC1/ | reverse | CT (SEQ ID NO: 9) |
| G6PD | Hydrolysis | AGA AGC CCG CCT CCA CCA ACT |
| | JA270/BHQ2- | CAG ATG AC (SEQ ID NO: 10) |
| | labeled | |
| | G6PD probe | |
| HPRT | Primer HPRT | GAC CTT GAT TTA TTT TGC ATA |
| duplex | forward | CCT A (SEQ ID NO: 11) |
| PCR | Primer HPRT | GAG CAA GAC GTT CAG TCC T |
| MACC1/ | reverse | (SEQ ID NO: 12) |
| HPRT | Hydrolysis | ATG CTG AGG ATT TGG AAA GGG |
| | JA270/BHQ2- | TGT TTA TTC (SEQ ID NO: 13) |
| | labeled | |
| | HPRT probe | |

### B. Experiments

Samples from four cohorts of patients were analyzed in this study using a variety of assays. An overview is given in Table 2. Abbreviations in Table 2 are as follows: Cryo, cryopreserved; dMMR, defective mismatch repair; FFPE, formalin-fixed paraffin-embedded; IHC, immunohistochemistry; MSI-H, microsatellite instability-high; MSS/MSI-L, microsatellite stable/microsatellite instability-low; MSI/MMR, microsatellite instability/mismatch repair; n.d., not done; pMMR, proficient mismatch repair; qRT-PCR, quantitative real-time polymerase chain reaction; UICC, Union for International Cancer Control.

TABLE 2
Overview and Purpose of Independently Studied Colorectal Patient Cohorts.

| | | Charité 1: | Charité 2: | | BIOGRID 2: |
|---|---|---|---|---|---|
| | | Discovery | Comparison | BIOGRID 1: | Validation |
| | | cohort | cohort | Training cohort | cohort |
| Patients | Total No | 61 | 40 | 189 | 306 |
| | UICC | I-III | I-III | II | II |
| | Stage | | | | |
| | Relapse | 14 | 20 | 48 | 31 |
| | (n) | | | | |
| | Treatment | No | No | No: 125 | No: 257 |
| | (n) | | | Yes: 64 | Yes: 49 |
| Sample (n) | Cryo | 61 | 40 | 0 | 0 |
| | FFPE | 0 | 40 | 189 | 306 |
| MACC1 | mRNA | 61 | 2 × 40 | 189 | n.d. |
| Expression | qRT-PCR | | | | |
| | Protein | n.d. | n.d. | 189 | 306 |
| | IHC | | | | |

| | | | | |
|---|---|---|---|---|
| MSI/MMR (n) | MSS/MSI-L: | n.d. | dMMR: 39 | dMMR: 54 |
| | 57 | | pMMR: 150 | pMMR: 252 |
| | MSI-H: 4 | | | |
| Aim of Study Cohort | Discovering | Comparing | Testing MACC1 | Validating |
| | benefit of | MACC1 mRNA | mRNA/protein | MACC1 |
| | combination of | levels in cryo | levels and MMR | protein levels |
| | MACC1 | and FFPE | status in FFPE | and MMR |
| | mRNA levels | samples for | samples for | status in FFPE |
| | and MSI status | prognosis | prognosis and | samples for |
| | in cryo samples | | prediction | prognosis and |
| | for prognosis | | | prediction |

B1. Discovery and Comparison Cohorts: Charité 1 and 2

The Charité 1 discovery cohort was used to evaluate if the combination of MACC1 mRNA expression level and microsatellite instability (MSI) status was prognostic. We analyzed cryo-preserved tumor tissues from patients with patho-histologically confirmed primary Union for International Cancer Control (UICC) stage I-III colorectal adenocarcinomas.

The Charité 2 comparison cohort was used to translate the prognostic importance of MACC1, as determined in cryo-preserved tissues, to analyses in formalin-fixed paraffin-embedded (FFPE) tissue samples. Therefore, we compared MACC1 mRNA expression in corresponding cryo-preserved tumor tissue and FFPE tumor tissue from patients with patho-histologically confirmed primary UICC stage I-III colorectal adenocarcinomas (without distant metastasis at the time of surgery).

B2. Test and Validation Cohorts: BIOGRID 1 and 2

The aim of the test and validation cohorts—BIOGRID 1 and BIOGRID 2, respectively—was to evaluate the prognostic and predictive value of MACC1 mRNA and protein expression combined with MMR status.

To simplify the use of biomarkers for routine pathology, we aimed to translate the expression of MACC1 mRNA levels determined by quantitative real-time polymerase chain reaction (qRT-PCR) to MACC1 protein levels determined by immunohistochemistry. We thus analyzed consecutive FFPE tissue sections from the same tumor for MACC1 mRNA and protein levels using qRT-PCR and immunohistochemistry, respectively. FFPE tissue was also analyzed by immunohistochemistry for MMR status. We used the BIOGRID 1 cohort with patho-histologically confirmed primary UICC stage II adenocarcinoma of the colon, enriched for recurrence of disease in 25% of patients to test this hypothesis. Findings from the BIOGRAD 1 cohort were then confirmed in the independent BIOGRID 2 cohort of unselected patients with stage II colon cancer.

### C. Results

Full details of the Charité 1 and 2 patient characteristics are given in Tables 3-5. Characteristics of the BIOGRID 1 and 2 patients are shown in Tables 6 and 7 and Table 1.

C1. Charité 1: Discovery Cohort

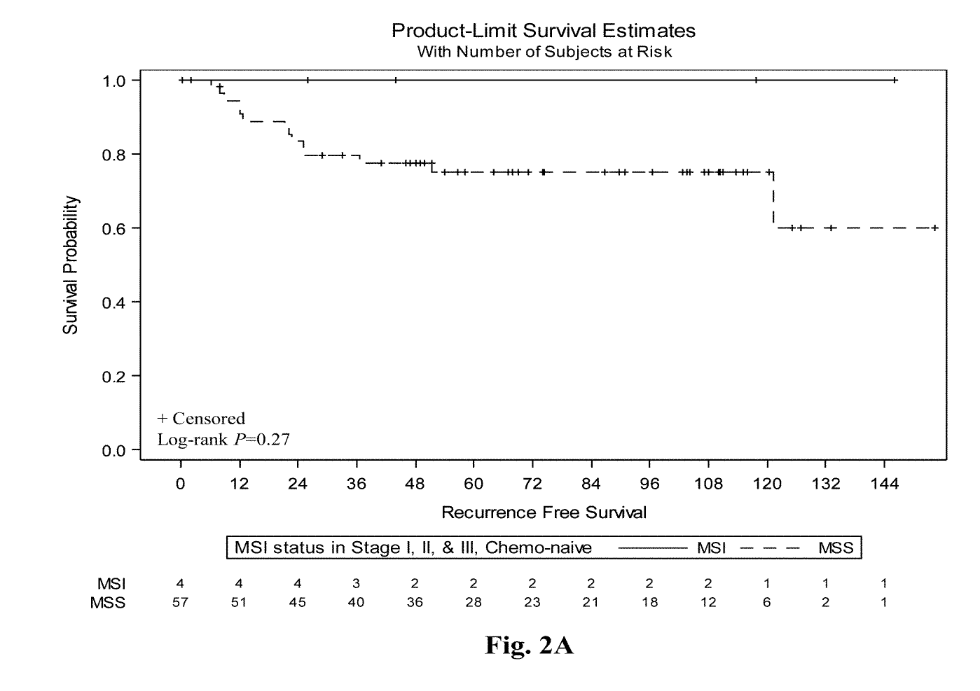

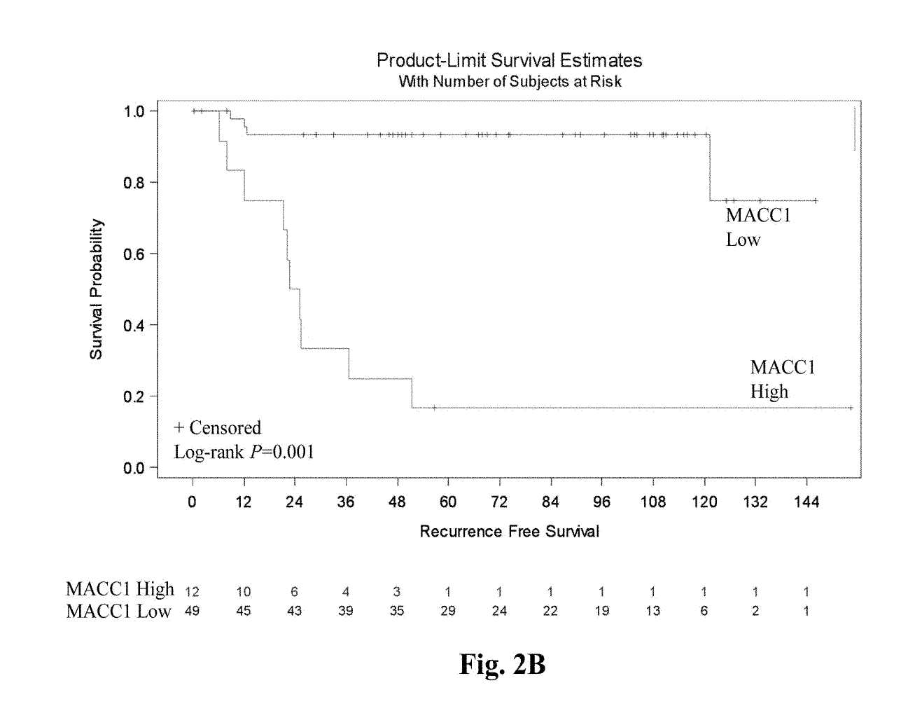

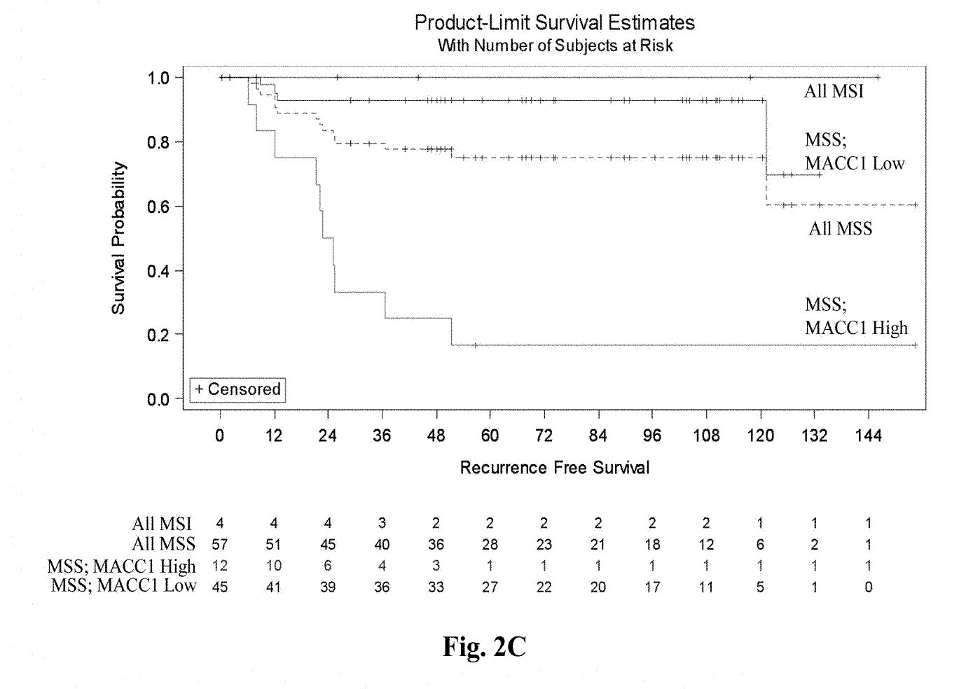

We initially evaluated MACC1 mRNA levels to aid MSI-based survival prognostication for patients with CRC in the Charité 1 cohort (Table 3). By applying the microsatellite stable (MSS)/MSI criteria, patients in the MSI-high (MSI-H) group had an inferior overall survival versus those the MSS/MSI-low (MSI-L) group. (FIG. 2B). MACC1 levels significantly correlated with patient survival (P<0.0001). The Kaplan-Meier curve for MSS/MSI-L patients separated by MACC1 level: patients with MSS/MSI-L/MACC1-low tumors showed significantly better survival versus those with MSS/MSI-L/MACC1-high (P<0.0001). Patients with MSS/MSI-L/MACC1-low tumors had a similar prognosis to patients with MSI-H tumors (FIGS. 2A-2C).

C2. Charité 2: Comparison Cohort

The Charité 2 cohort was used to assess if, when using qRT-PCR for MACC1 detection, changing from cryo-preserved to FFPE tissue samples affected the test results by analyzing corresponding tissue samples from the same patients (Tables 4 and 5). MACC1 expression was significantly higher in metachronously metastasizing tumors linked to shorter relapse-free survival (RFS), independent of the tissue type analyzed (cryo-preserved or FFPE) and of the housekeeping gene(s) used for normalization: MACC1 exclusively, MACC1/glucose-6-phosphate-dehydrogenase (G6PD), MACC1/hypoxanthine phosphoribosyltransferase1 (HPRT1), or MACC1/G6PD+HPRT (FIGS. 3A-H).

TABLE 3
Patient Characteristics and MACC1 mRNA Expression in
Cryo-preserved Tissues from the Charité 1 Cohort
(#: one case with missing detailed information about
the localization within the colon or rectum)

MACC1 mRNA Expression

| Characteristic | Low (n = 49) | | High (n = 12) | |
|---|---|---|---|---|
| Sex | | | | |
| Male (n = 36) | 27 | (55.1%) | 9 | (75.0%) |
| Female (n = 25) | 22 | (44.9%) | 3 | (25.0%) |
| Age at diagnosis | | | | |
| <60 years (n = 14) | 12 | (24.5%) | 2 | (16.7%) |
| ≥60 years (n = 47) | 37 | (75.5%) | 10 | (83.3%) |
| UICC stage | | | | |
| I (n = 22) | 19 | (38.8%) | 3 | (25.0%) |
| II (n = 26) | 21 | (42.9%) | 5 | (41.7%) |
| III (n = 13) | 9 | (18.4%) | 4 | (33.3%) |
| pT category | | | | |
| pT 1/2 (n = 25) | 21 | (42.9%) | 4 | (33.3%) |
| pT 3/4 (n = 36) | 28 | (57.1%) | 8 | (66.7%) |
| pN category | | | | |
| Negative (n = 48) | 40 | (81.6%) | 8 | (66.7%) |
| Positive (n = 13) | 9 | (18.4%) | 4 | (33.3%) |
| Grading | | | | |
| Grade 1/2 (n = 49) | 39 | (79.6%) | 10 | (83.3%) |
| Grade 3/4 (n = 12) | 10 | (20.4%) | 2 | (16.7%) |
| Localization^{#} | | | | |
| Colon (n = 39) | 35 | (71.4%) | 4 | (33.3%) |
| Rectum (n = 21) | 13 | (26.5%) | 8 | (66.7%) |
| Relapse during follow-up | | | | |
| period | | | | |
| Relapse-free (n = 47) | 45 | (91.8%) | 2 | (16.7%) |
| Relapse (n = 14) | 4 | (8.2%) | 10 | (83.3%) |
| MSI status | | | | |
| MSS/MSI-L (n = 57) | 45 | (91.8%) | 12 | (100.0%) |
| MSI-H (n = 4) | 4 | (8.2%) | 0 | (0.0%) |

TABLE 4
Patient Characteristics and MACC1 mRNA Expression in Cryo-preserved Tissues
from the Charite 2 cohort

| | | | MACC1 mRNA | | | | MACC1 mRNA | |
|---|---|---|---|---|---|---|---|---|
| | | | Expression | | MACC1 mRNA | | Expression | |
| | MACC1 mRNA | | Normalized v | | Expression | | Normalized v | |
| | Expression | | G6PD | | Normalized v HPRT | | G6PD + HPRT | |
| | Low | High | Low | High | Low | High | Low | High |
| Characteristic | (n = 18) | (n = 22) | (n = 21) | (n = 19) | (n = 27) | (n = 13) | (n = 24) | (n = 16) |
| Sex | | | | | | | | |
| Male | 12 | 12 | 13 | 11 | 16 | 8 | 15 | 9 |
| (n = 24) | | | | | | | | |
| Female | 6 | 10 | 8 | 8 | 11 | 5 | 9 | 7 |
| (n = 16) | | | | | | | | |
| Age at | | | | | | | | |
| diagnosis | | | | | | | | |
| <60 years | 11 | 6 | 13 | 4 | 15 | 2 | 14 | 3 |
| (n = 17) | | | | | | | | |
| ≥60 years | 7 | 16 | 8 | 16 | 12 | 11 | 10 | 13 |
| (n = 23) | | | | | | | | |
| UICC stage | | | | | | | | |
| I (n = 12) | 7 | 5 | 6 | 6 | 8 | 4 | 7 | 5 |
| II (n = 17) | 6 | 11 | 8 | 9 | 10 | 7 | 8 | 9 |
| III (n = 11) | 5 | 6 | 7 | 4 | 9 | 2 | 9 | 2 |
| pT category | | | | | | | | |
| pT1/2 | 8 | 5 | 7 | 6 | 9 | 4 | 8 | 5 |
| (n = 13) | | | | | | | | |
| pT3/4 | 10 | 17 | 14 | 13 | 18 | 9 | 16 | 11 |
| (n = 27) | | | | | | | | |
| pN category | | | | | | | | |
| Negative | 13 | 16 | 14 | 15 | 18 | 11 | 15 | 14 |
| (n = 29) | | | | | | | | |
| Positive | 5 | 6 | 7 | 4 | 9 | 2 | 9 | 2 |
| (n = 11) | | | | | | | | |
| Grading | | | | | | | | |
| Grade 1/2 | 13 | 18 | 16 | 15 | 21 | 10 | 19 | 12 |
| (n = 31) | | | | | | | | |
| Grade 3/4 | 5 | 4 | 5 | 4 | 6 | 3 | 5 | 4 |
| (n = 9) | | | | | | | | |
| Localization | | | | | | | | |
| Colon | 12 | 10 | 14 | 8 | 16 | 6 | 14 | 8 |
| (n = 22) | | | | | | | | |
| Rectum | 6 | 12 | 7 | 11 | 11 | 7 | 10 | 8 |
| (n = 18) | | | | | | | | |
| Relapse | | | | | | | | |
| during | | | | | | | | |
| follow-up | | | | | | | | |
| period | | | | | | | | |
| Relapse- | 16 | 4 | 15 | 5 | 17 | 3 | 16 | 4 |
| free (n = 20) | | | | | | | | |
| Relapse | 2 | 18 | 6 | 14 | 10 | 10 | 8 | 12 |
| (n = 20) | | | | | | | | |

TABLE 5
Patient characteristics and MACC1 mRNA expression in FFPE tumor
tissues from the Charité 2 cohort (FFPE, formalin-fixed paraffin-embedded; G6PD,
glucose-6-phosphate-dehydrogenase gene; HPRT, hypoxanthine
phosphoribosyltransferase 1 gene; pN, post-operative lymph node status; pT, post-
operative tumor stage; UICC, Union for International Cancer Control.)

| | | | MACC1 mRNA | | | | MACC1 mRNA | |
|---|---|---|---|---|---|---|---|---|
| | | | Expression | | MACC1 mRNA | | Expression | |
| | MACC1 mRNA | | Normalized v | | Expression | | Normalized v | |
| | Expression | | G6PD | | Normalized v HPRT | | G6PD + HPRT | |
| | Low | High | Low | High | Low | High | Low | High |
| Characteristic | (n = 29) | (n = 11) | (n = 19) | (n = 21) | (n = 19) | (n = 21) | (n = 24) | (n = 16) |
| Sex | | | | | | | | |
| Male | 17 | 7 | 10 | 14 | 11 | 13 | 13 | 11 |
| (n = 24) | | | | | | | | |
| Female | 12 | 4 | 9 | 7 | 8 | 8 | 11 | 5 |
| (n = 16) | | | | | | | | |
| Age at | | | | | | | | |
| diagnosis | | | | | | | | |
| <60 years | 15 | 2 | 11 | 6 | 10 | 7 | 12 | 5 |
| (n = 17) | | | | | | | | |
| ≥60 years | 14 | 9 | 8 | 15 | 9 | 14 | 12 | 11 |
| (n = 23) | | | | | | | | |
| UICC stage | | | | | | | | |
| I (n = 12) | 10 | 2 | 3 | 9 | 4 | 8 | 7 | 5 |
| II (n = 17) | 12 | 5 | 7 | 10 | 7 | 10 | 8 | 9 |
| III (n = 11) | 7 | 4 | 9 | 2 | 8 | 3 | 9 | 2 |
| pT status | | | | | | | | |
| pT1/2 | 10 | 3 | 4 | 9 | 5 | 8 | 8 | 5 |
| (n = 13) | | | | | | | | |
| pT3/4 | 19 | 8 | 15 | 12 | 14 | 13 | 16 | 11 |
| (n = 27) | | | | | | | | |
| pN status | | | | | | | | |
| Negative | 22 | 7 | 10 | 19 | 11 | 18 | 15 | 14 |
| (n = 29) | | | | | | | | |
| Positive | 7 | 4 | 9 | 2 | 8 | 3 | 9 | 2 |
| (n = 11) | | | | | | | | |
| Grading | | | | | | | | |
| Grade 1/2 | 22 | 9 | 13 | 18 | 12 | 19 | 17 | 14 |
| (n = 31) | | | | | | | | |
| Grade 3/4 | 7 | 2 | 6 | 3 | 7 | 2 | 7 | 2 |
| (n = 9) | | | | | | | | |
| Localization | | | | | | | | |
| # | | | | | | | | |
| Colon | 17 | 5 | 13 | 9 | 13 | 9 | 14 | 8 |
| (n = 22) | | | | | | | | |
| Rectum | 12 | 6 | 6 | 12 | 6 | 12 | 10 | 8 |
| (n = 18) | | | | | | | | |
| Relapse | | | | | | | | |
| during | | | | | | | | |
| follow-up | | | | | | | | |
| period | | | | | | | | |
| Relapse- | 18 | 2 | 12 | 8 | 12 | 8 | 15 | 5 |
| free (n = 20) | | | | | | | | |
| Relapse | 11 | 9 | 7 | 13 | 7 | 13 | 9 | 11 |
| (n = 20) | | | | | | | | |

TABLE 6
Patient Characteristics and MACC1 mRNA and Protein Expression in FFPE
Tissues from the BIOGRID 1 Cohort (FFPE, formalin-fixed paraffin-embedded;
MACC1, Metastasis Associated in Colon Cancer 1; MMR, mismatch repair;
pN, post-operative lymph node status; pT, post-operative tumor stage; UICC,
Union for International Cancer Control.)

| | MACC1 mRNA Expression | | | | MACC1 Protein Expression | | | |
|---|---|---|---|---|---|---|---|---|
| Characteristic | Low (n = 27) | | High (n = 162) | | Low (n = 16) | | High (n = 173) | |
| Sex | | | | | | | | |
| Male (n = 94) | 12 | (44.4%) | 82 | (50.6%) | 9 | (56.3%) | 85 | (49.1%) |
| Female (n = 95) | 15 | (55.6%) | 80 | (49.4%) | 7 | (43.8%) | 88 | (50.9%) |
| Age at diagnosis | | | | | | | | |
| <60 years (n = 44) | 8 | (29.6%) | 36 | (22.2%) | 2 | (12.5%) | 42 | (24.3%) |
| ≥60 years (n = 145) | 19 | (70.4%) | 126 | (77.8%) | 14 | (87.5%) | 131 | (75.7%) |
| UICC stage | | | | | | | | |
| Stage II (n = 189) | 27 | (100.0%) | 162 | (100.0%) | 16 | (100.0%) | 173 | (100.0%) |
| pT category | | | | | | | | |
| pT3 (n = 159) | 23 | (85.2%) | 136 | (84.0%) | 12 | (75.0%) | 147 | (85.0%) |
| pT4 (n = 30) | 4 | (14.8%) | 26 | (16.0%) | 4 | (25.0%) | 26 | (15.0%) |
| pN category | | | | | | | | |
| Negative (n = 189) | 27 | (100.0%) | 162 | (100.0%) | 16 | (100.0%) | 173 | (100.0%) |
| Grading | | | | | | | | |
| Grade 1/2 (n = 145) | 23 | (85.2%) | 122 | (75.3%) | 14 | (87.5%) | 131 | (75.7%) |
| Grade 3/4 (n = 41) | 4 | (14.8%) | 37 | (22.8%) | 1 | (6.3%) | 40 | (23.1%) |
| Missing (n = 3) | 0 | | 3 | (1.9%) | 1 | (6.3%) | 2 | (1.2%) |
| Localization | | | | | | | | |
| Colon (n = 189) | 27 | (100.0%) | 162 | (100.0%) | 16 | (100.0%) | 173 | (100.0%) |
| Relapse during | | | | | | | | |
| follow-up period | | | | | | | | |
| Relapse-free (n = 141) | 23 | (85.2%) | 118 | (72.8%) | 16 | (100%) | 125 | (72.3%) |
| Relapse (n = 48) | 4 | (14.8%) | 44 | (27.2%) | 0 | | 48 | (27.7%) |
| MMR status | | | | | | | | |
| Proficient (n = 150) | 20 | (74.1%) | 130 | (80.2%) | 11 | (68.8%) | 139 | (80.3%) |
| Deficient (n = 39) | 7 | (25.9%) | 32 | (19.8%) | 5 | (31.2%) | 34 | (19.7%) |

TABLE 7
Patient Characteristics and MACC1 Protein Expression in
FFPE Tissues from the BIOGRID 2 Cohort (FFPE, formalin-
fixed paraffin-embedded; MACC1, Metastasis Associated
in Colon Cancer 1; MMR, mismatch repair; pN, post-operative
lymph node status; pT, post-operative tumor stage; UICC,
Union for International Cancer Control.)

MACC1 Protein Expression

| Characteristic | Low (n = 16) | | High (n = 290) | |
|---|---|---|---|---|
| Sex | | | | |
| Male (n = 175) | 7 | (43.8%) | 168 | (57.9%) |
| Female (n = 131) | 9 | (56.3%) | 122 | (42.1%) |
| Age at diagnosis | | | | |
| <60 years (n = 41) | 0 | | 41 | (14.1%) |
| ≥60 years (n = 265) | 16 | (100.0%) | 249 | (85.9%) |
| UICC stage | | | | |
| Stage II (n = 306) | 16 | (100.0%) | 290 | (100.0%) |
| pT category | | | | |
| pT3 (n = 261) | 13 | (81.3%) | 248 | (85.5%) |
| pT4 (n = 45) | 3 | (18.8%) | 42 | (14.5%) |
| pN category | | | | |
| Negative (n = 306) | 16 | (100.0%) | 290 | (100.0%) |
| Grading | | | | |
| Grade 1/2 (n = 236) | 11 | (68.8%) | 225 | (77.6%) |
| Grade 3/4 (n = 66) | 5 | (31.3%) | 61 | (21.0%) |
| Missing (n = 4) | 0 | | 4 | (1.4%) |
| Localization | | | | |
| Colon (n = 306) | 16 | (100.0%) | 290 | (100.0%) |
| Relapse during | | | | |
| follow-up period | | | | |
| Relapse-free (n = 275) | 16 | (100.0%) | 259 | (89.3%) |
| Relapse (n = 31) | 0 | | 31 | (10.7%) |
| MMR status | | | | |
| Proficient (n = 252) | 12 | (75.0%) | 240 | (82.8%) |
| Deficient (n = 54) | 4 | (25.0%) | 50 | (17.2%) |

C3. BIOGRID 1: Test Cohort

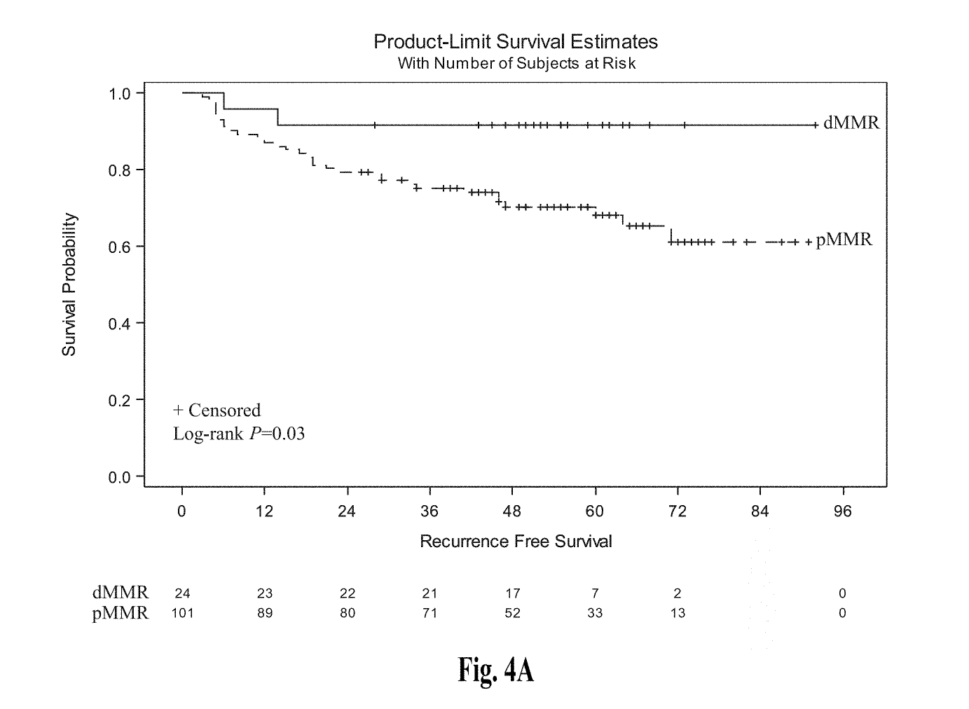

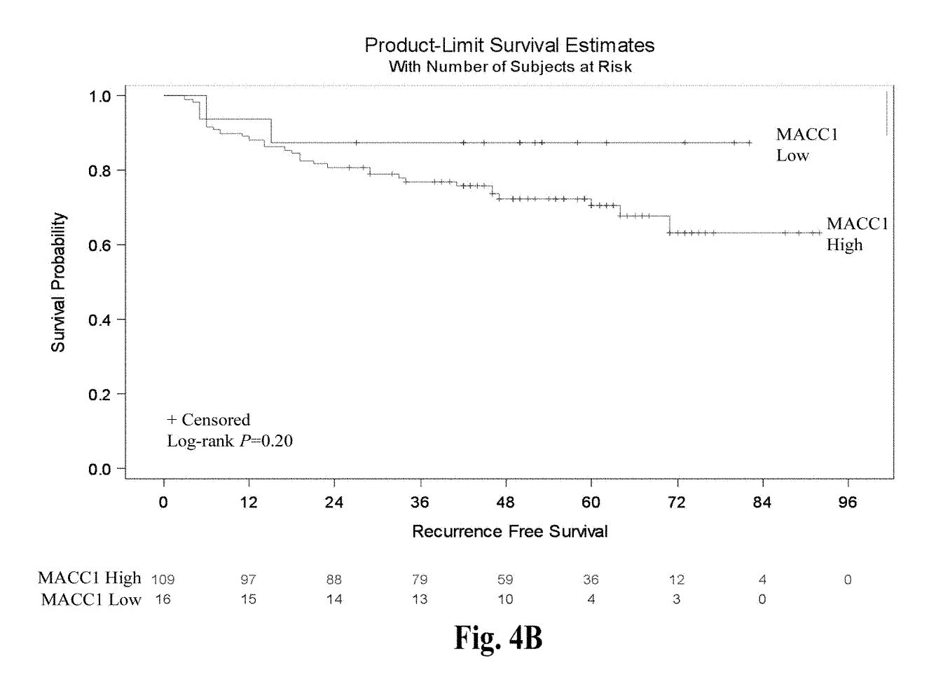

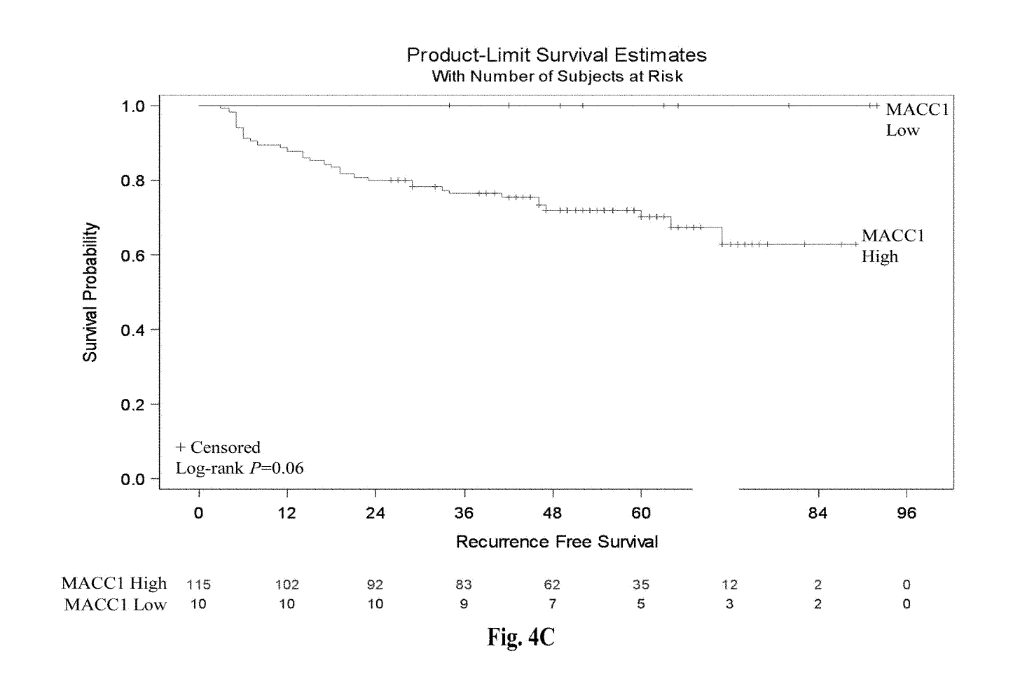

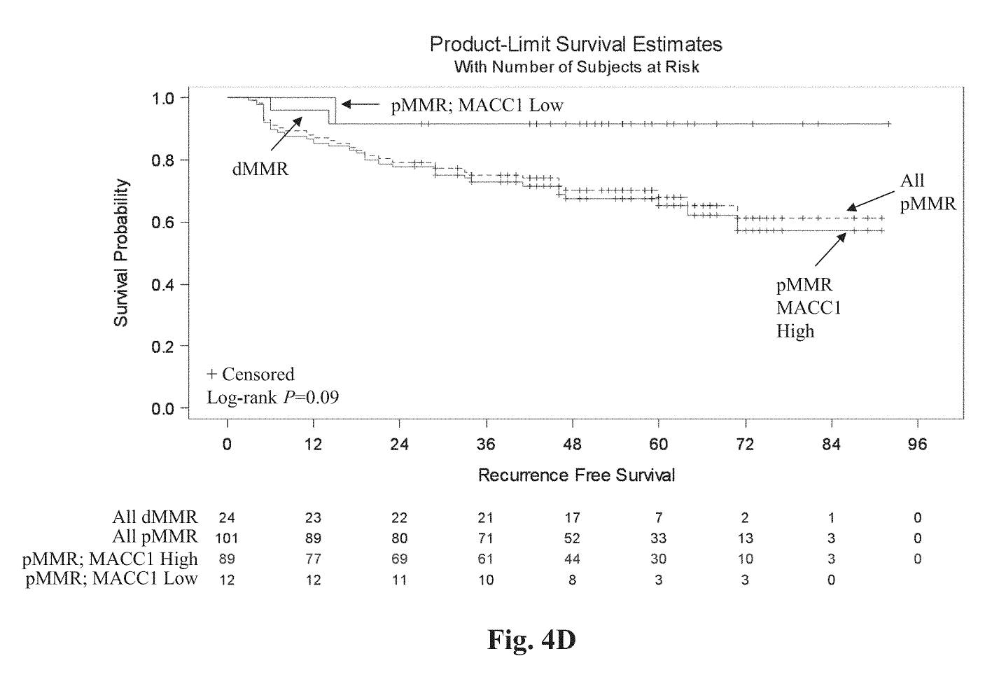

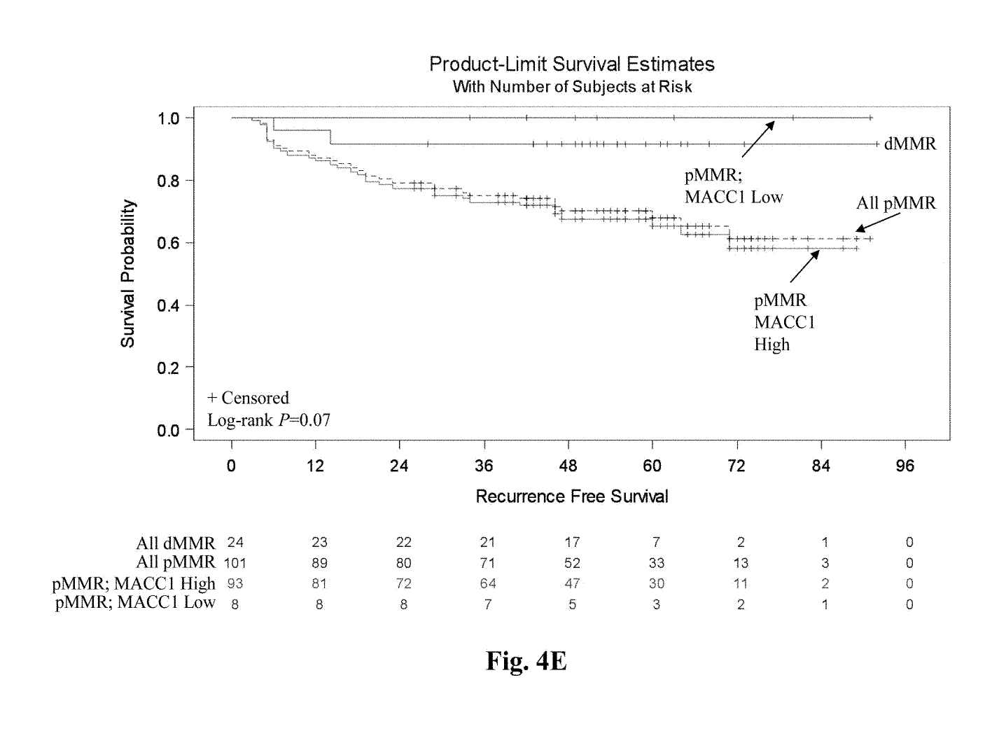

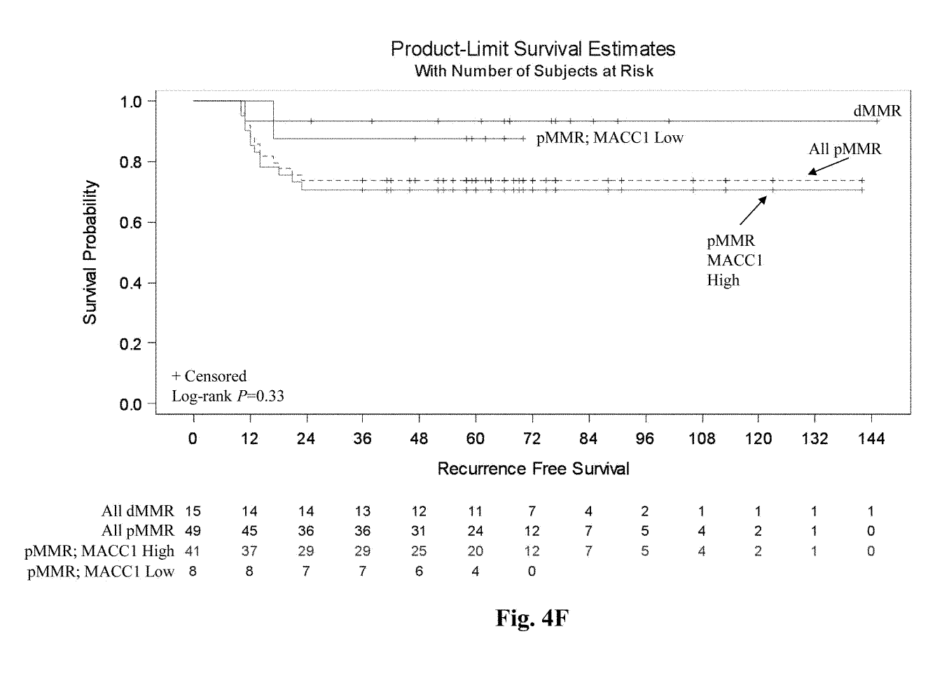

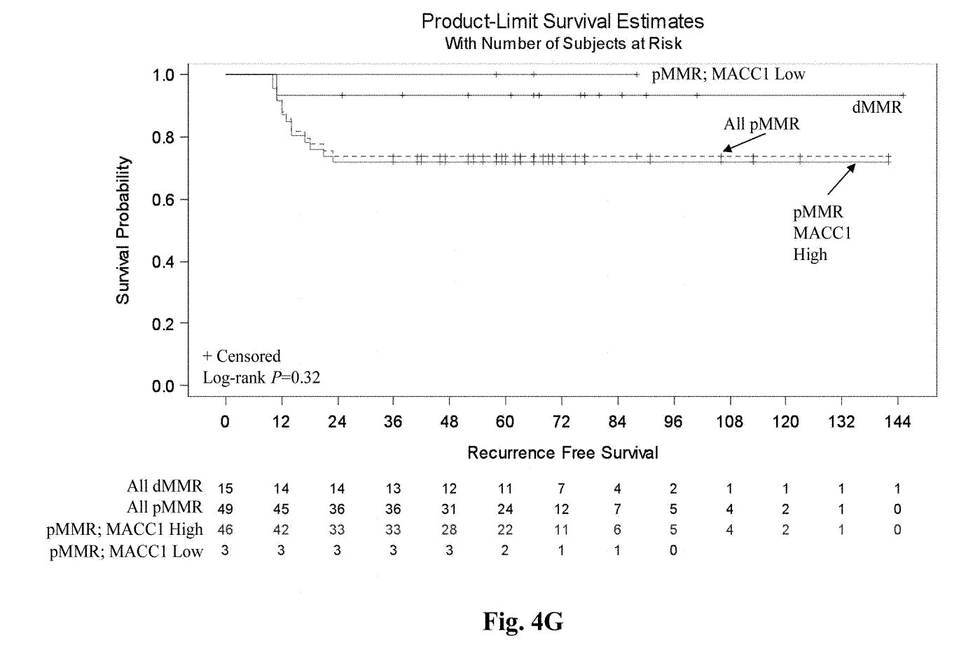

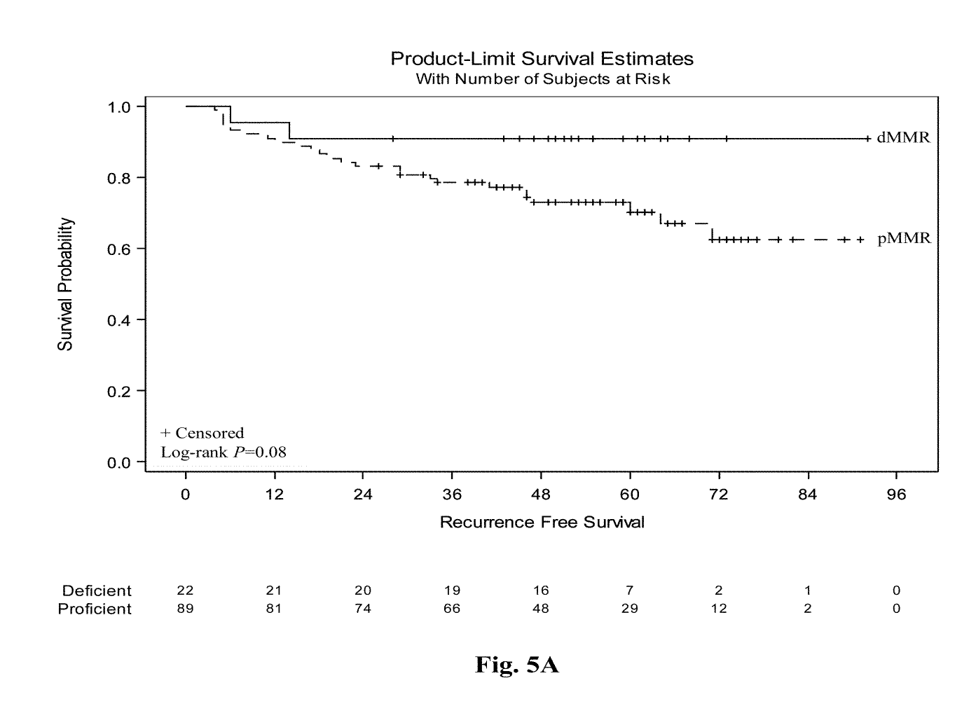

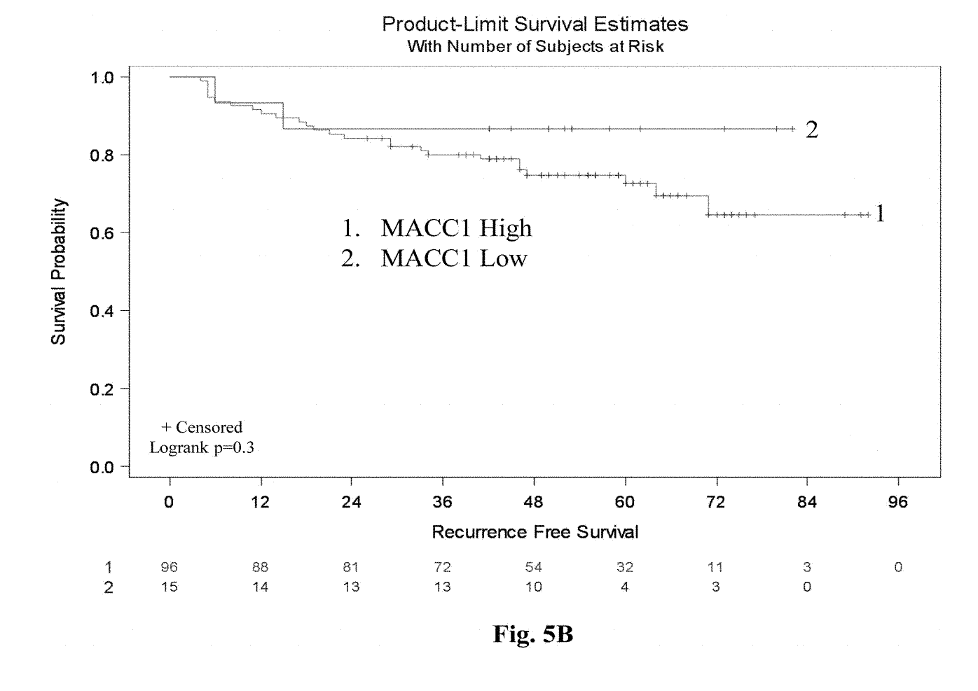

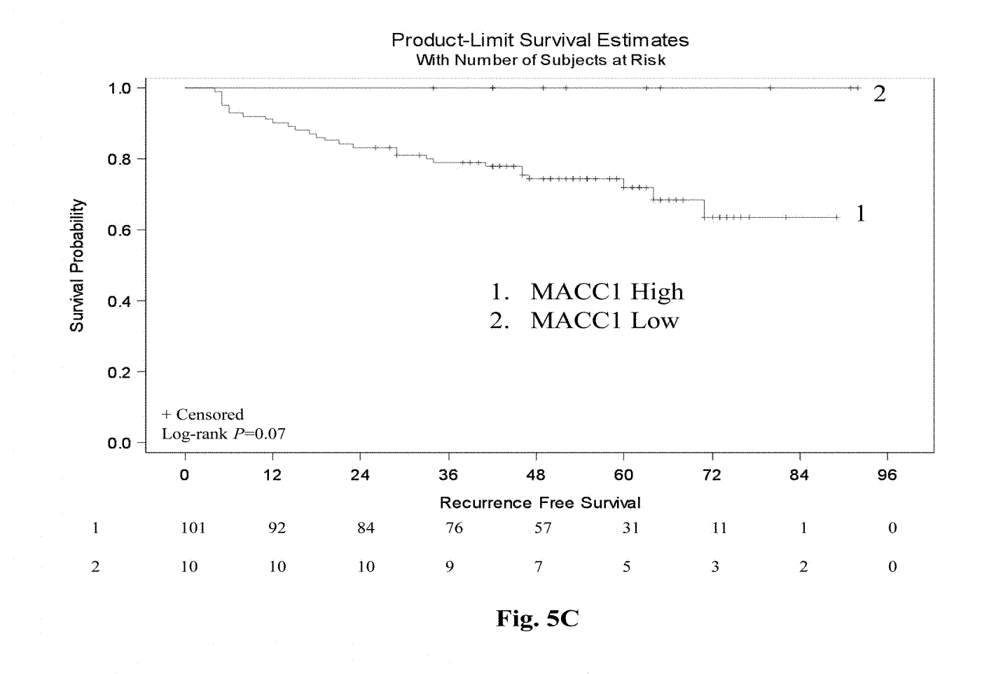

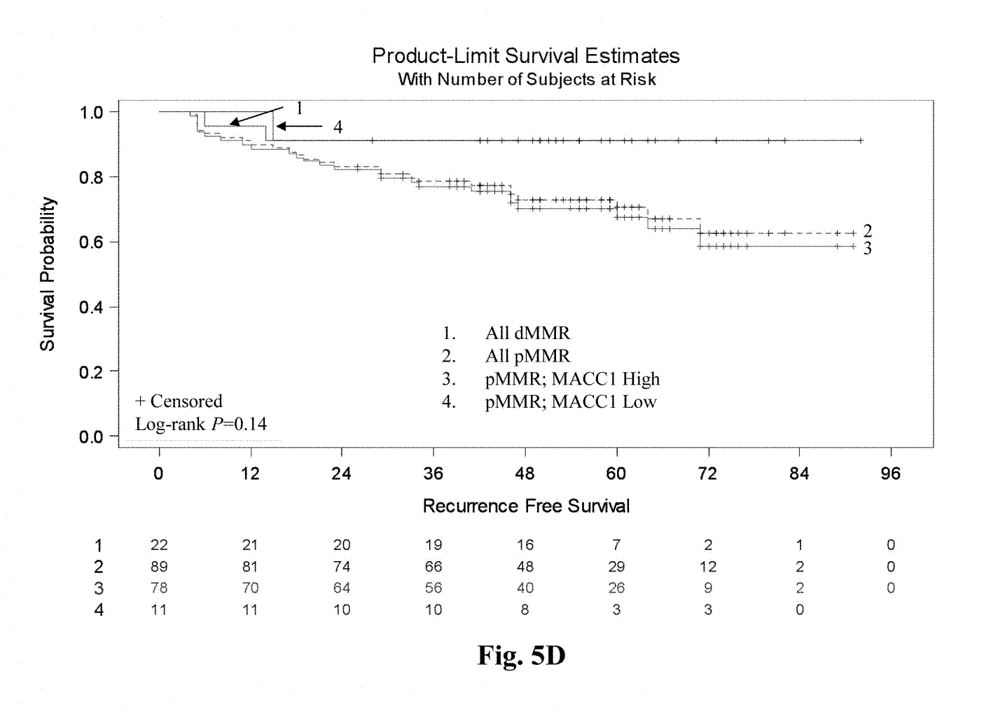

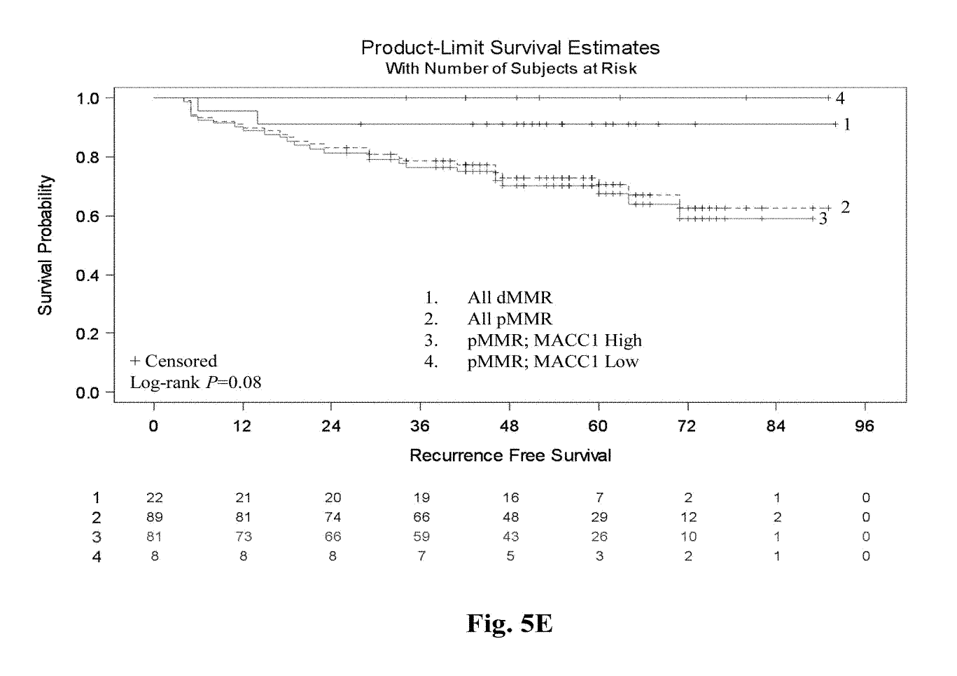

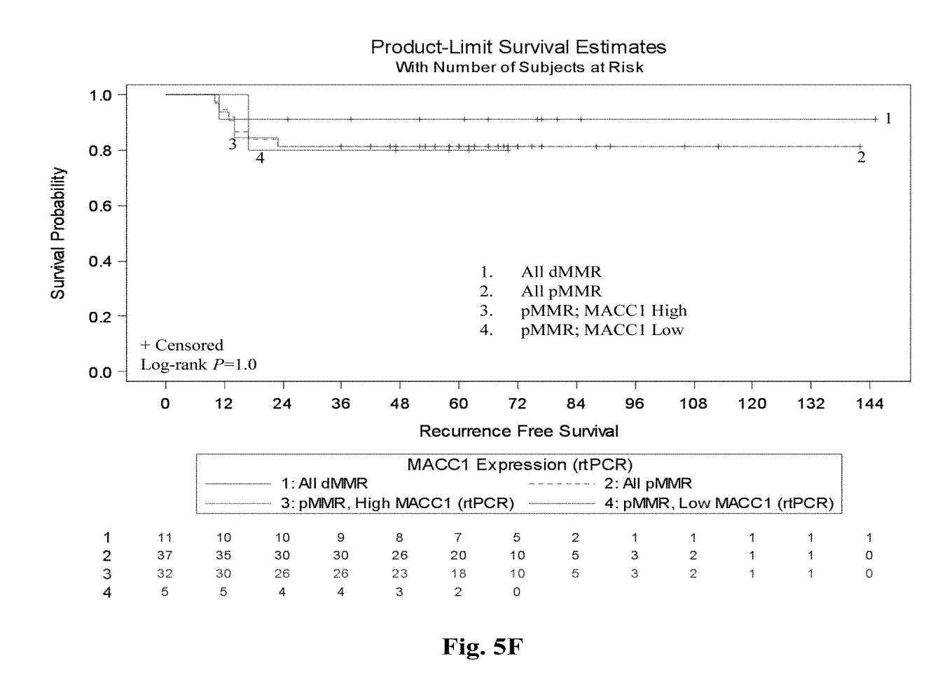

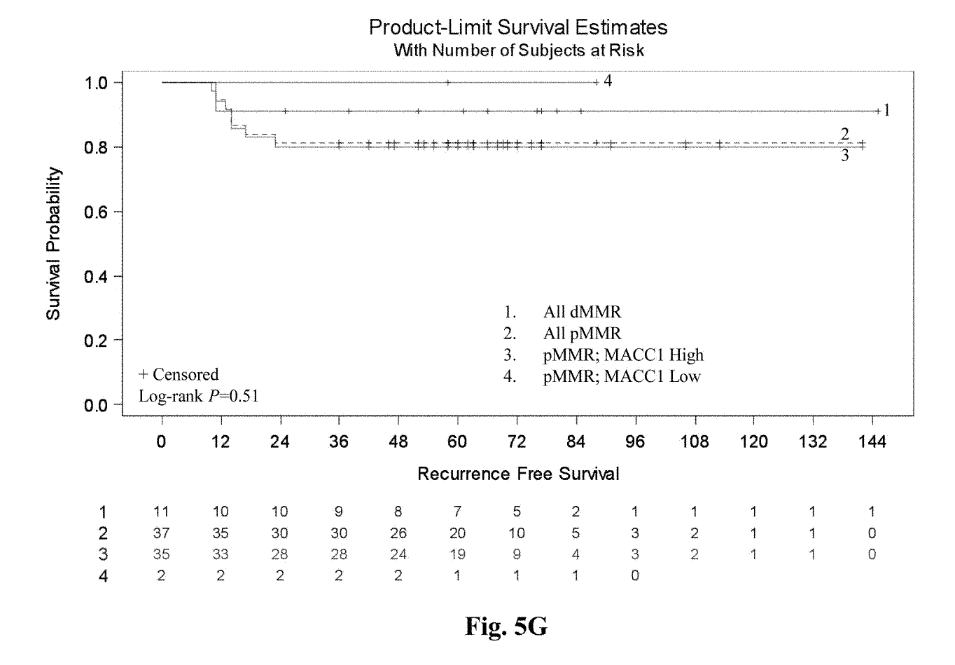

Initially, owing to the potential confounding effect of adjuvant chemotherapy and the small number of chemotherapy-treated patients, we only analyzed patients who did not receive adjuvant therapy. We observed a significant separation of RFS between chemotherapy-naïve patients with dMMR and pMMR stage II colon cancer (P=0.03; FIG. 4A). For patients with stage II T3 disease, we also found a trend toward improved RFS for the dMMR versus pMMR group (P=0.08; FIG. 5A).

Next, we determined MACC1 mRNA levels using qRT-PCR in chemotherapy-naïve patients with stage II T3/T4 colon cancer. Patients with high MACC1 mRNA expression levels in their primary tumors showed a shorter RFS versus those with low levels (FIG. 3B; T3 only FIG. 4B).

When combining MMR status and MACC1 expression level in chemotherapy-naïve patients with pMMR status, those with low MACC1 mRNA tumor expression demonstrated a trend toward better RFS versus those with high MACC1 mRNA expression (P=0.09; FIG. 4D). This observation was also made in patients with stage II T3 disease (FIG. 5D).

Of relevance, 12% of patients (12/101) were identified in the MACC1 low group with pMMR status, who had a favorable prognosis. These patients with pMMR/MACC1 low showed comparable RFS to dMMR patients.

Samples from 64 patients who had been treated with fluoropyrimidine-based chemotherapy in the adjuvant setting were also tested. The pMMR/MACC1 low phenotype was found in 16% of patients (8/49). In the limited numbers of chemotherapy-treated patients, those with pMMR/MACC1 high tumors had a trend toward a worse prognosis compared with patients who had pMMR/MACC1 low tumors (FIG. 4F; FIG. 5F). In summary, a total of 13% of pMMR patients (20/150) in BIOGRID 1 had the pMMR/MACC1 low phenotype and an RFS similar to patients with dMMR tumors.

Next, the same tumor samples were again analyzed for MACC1 levels but a different technology for MACC1 detection was used. MACC1 protein levels were quantified using an immunohistochemistry scoring algorithm. Chemotherapy-naïve patients with >75% of cells staining for MACC1 protein expression (MACC1 high) in cells from their primary tumors showed shorter RFS versus patients with ≤75% of cells staining for MACC1 expression (P=0.06 for T3/T4 tumors (FIG. 4C); P=0.07 for T3 tumors alone (FIG. 5C)).

As before, we then analyzed the prognostic value for RFS of the combination of MMR status and MACC1 level in chemotherapy-naïve patients. Despite the use of a different technology for MACC1 expression on a protein level, adding MACC1 to the MMR status resulted in a similar separation of the pMMR Kaplan-Meier curves: patients with pMMR status and low MACC1 protein staining (MACC1 low) in their tumors demonstrated better RFS than patients with pMMR status and high MACC1 protein (MACC1 high) (P=0.07; FIG. 4E). For patients with stage II T3 disease, similar trends were obtained (P=0.08; FIG. 5E). Furthermore, the 8% of patients in the pMMR/MACC1 low group (8/101) had favorable survival in terms of RFS, which was of the same magnitude as for patients with dMMR tumors.

In the few chemotherapy-treated patients available for analysis, a non-significant separation of the curves was observed between the pMMR/MACC1 low and the pMMR/MACC1 high groups (6% [3/49] v 94% [46/49], respectively; FIG. 4G; FIG. 5G). In summary, 7% of patients' tumors (11/150) had the pMMR/MACC1 low phenotype.

C4. BIOGRID 2: Validation Cohort

To validate the identification of a pMMR/MACC1 low group with similar disease behavior as the dMMR status group, the BIOGRID 2 cohort of 306 patients with stage II colon cancer was analyzed using the previously established immunohistochemical technology with the same cutoff for MACC1 high and low protein expression (Table 7).

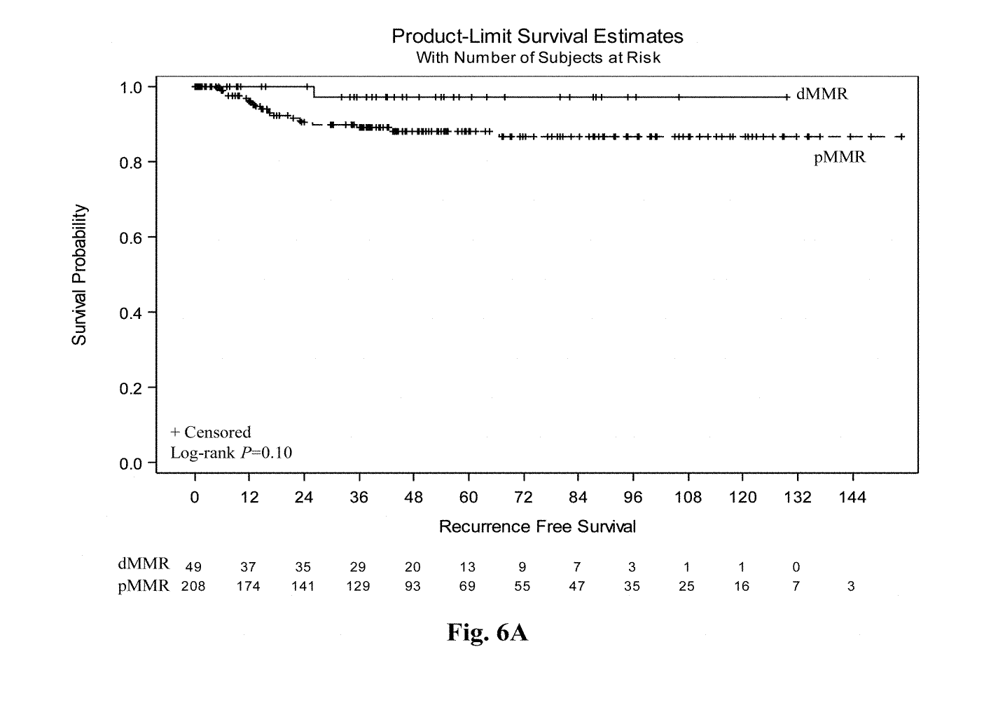

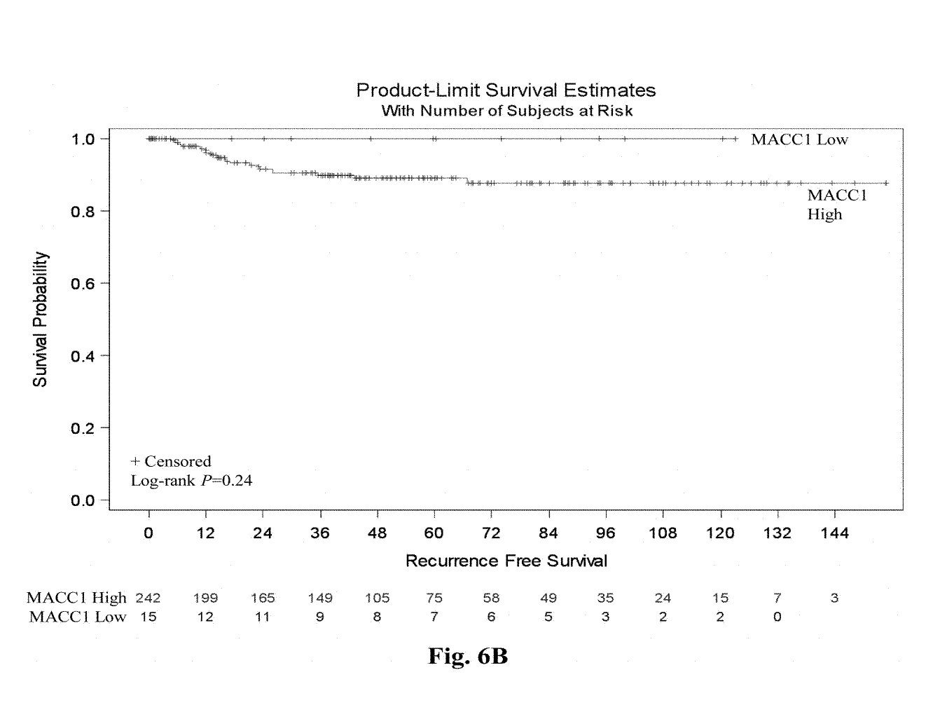

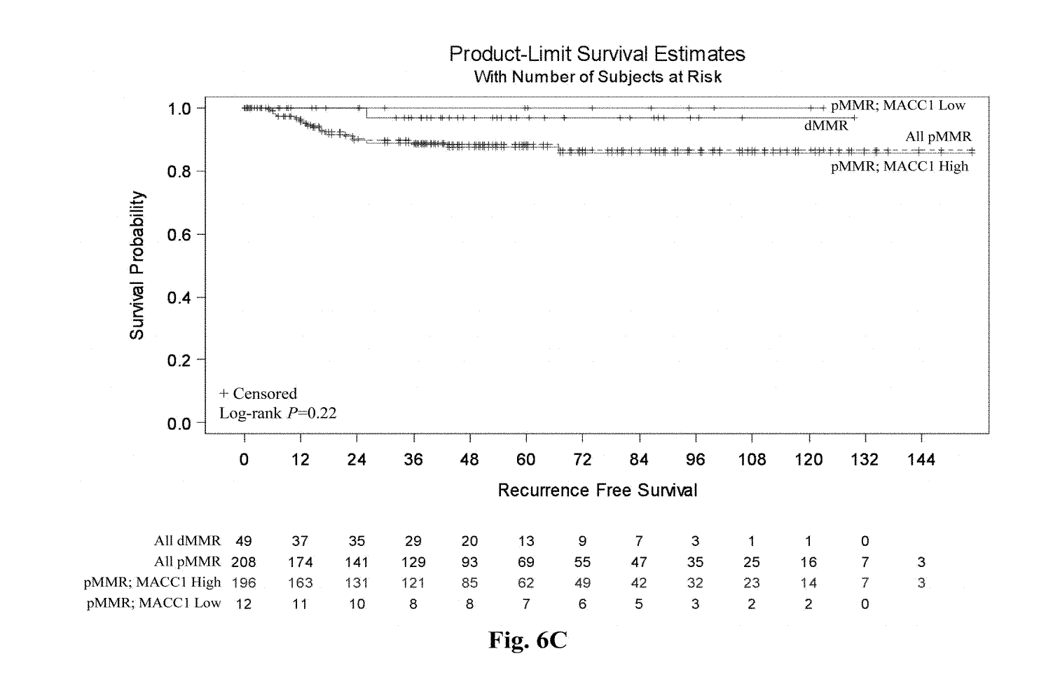

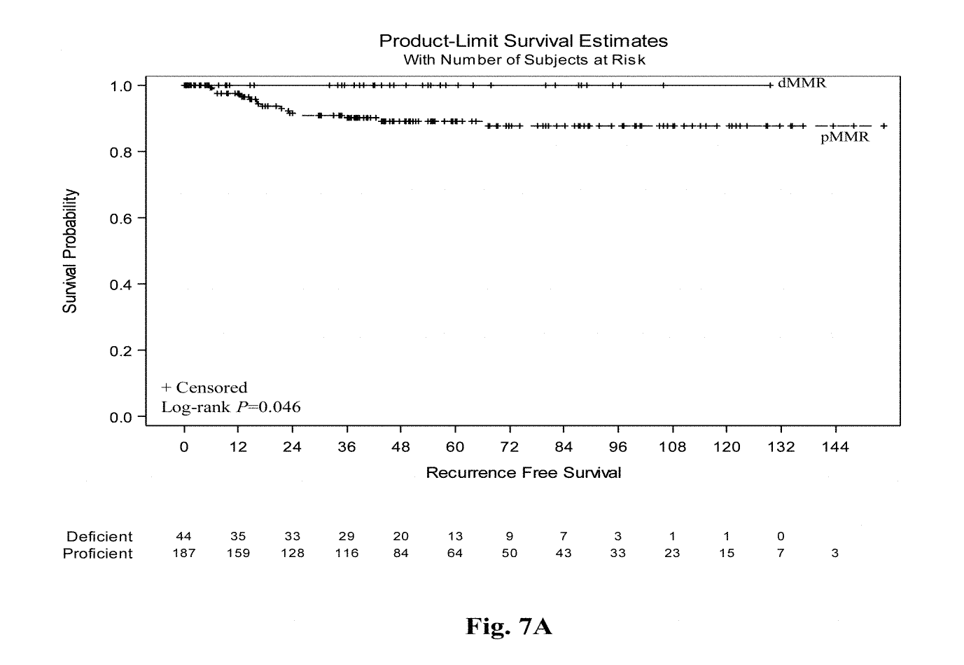

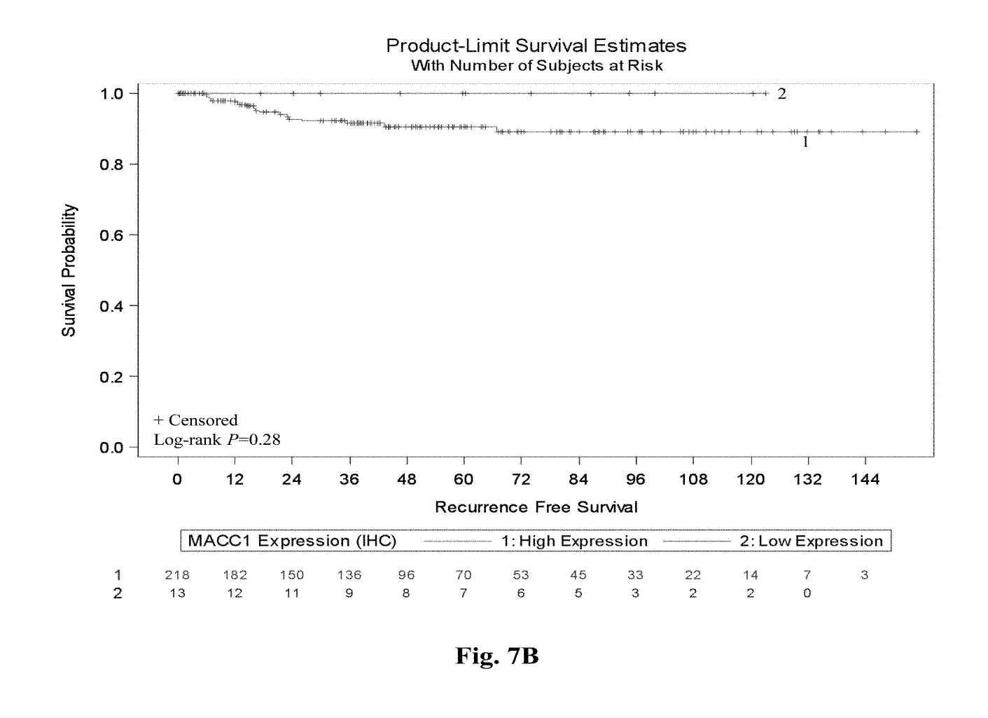

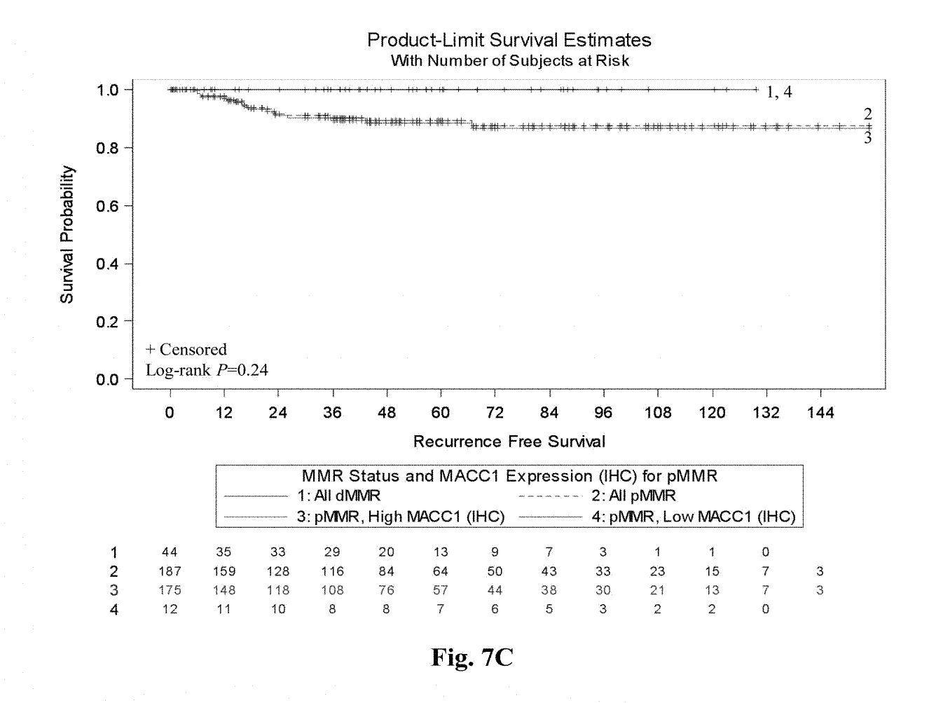

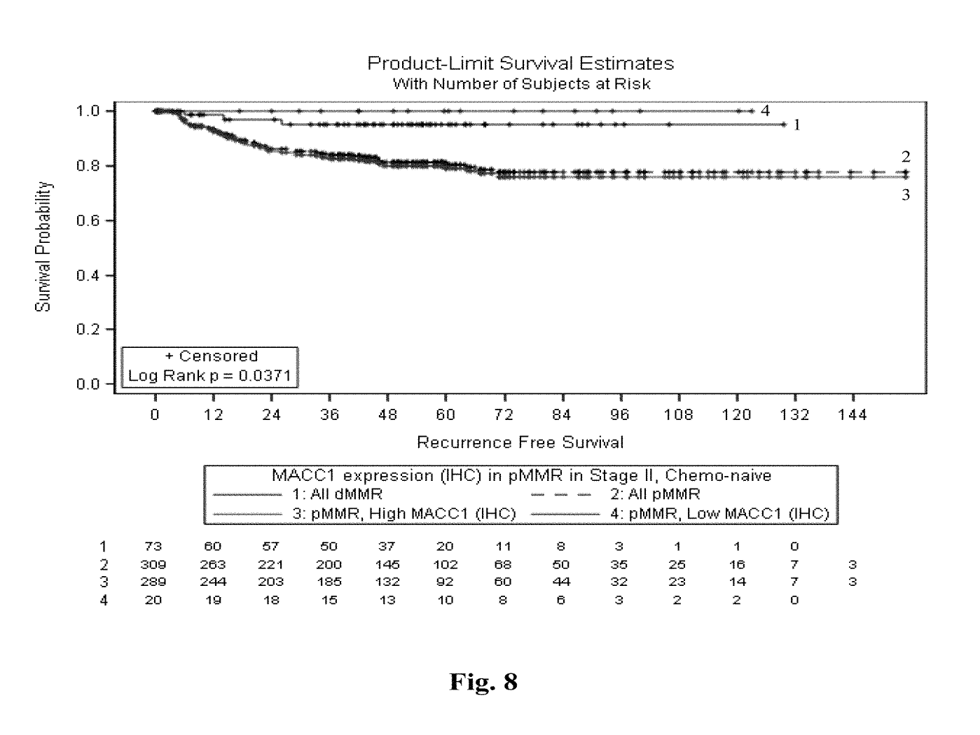

In the BIOGRID 2 cohort, as in BIOGRID 1, we observed RFS separation by MMR status (FIG. 5A; P=0.10; FIG. 6A) and by MACC1 level (high versus low FIG. 5B; FIG. 7B)) in chemotherapy-naïve patients with colon cancer. When analyzing patients with pMMR status according to MACC1 status, the RFS curves separated further (FIG. 5C; FIG. 7C). As in BIOGRID 1, we also identified in this unselected BIOGRID 2 cohort a pMMR/MACC1 low group of 6% of patients (12/208) with a favorable outcome clinically similar to the known favorable dMMR population.

In summary, in BIOGRID 2, 5% of patients (13/253) had the pMMR/MACC1 low phenotype. None of these patients progressed, showing a similar biologic behavior to patients with dMMR status.

C5. BIOGRID 1 and BIOGRID 2 Pooled

As the Sample size was not statistically derived for Biogrid 1 and 2 no estimate of effect size was available prior to this study, as this study was a first attempt to determine if a potentially relevant clinical association with MACC1 expression existed. For this reason, caution was taken not to rely on p-values alone as indicative of potential utility. After assessment of stratification in each cohort, cohorts B1 and B2 were pooled to obtain the most precise estimate of MACC1 informatively. Pooling of patients in Biogrid 1 and 2 significantly separates chemo naïve pMMR patients using MACC1 IHC. 6% of patients with pMMR/MACC1 low (20/309) did not have a recurrence of disease with a significant better mean RFS of 100% as compared to patients with pMMR/MACC1 high (p=0.037, FIG. 8). Thus, none of the pMMR/MACC1 low patients had a recurrence of disease suggesting a similar favorable outcome as those patients that are pMMR that do not derive benefit from fluoropyrimidine adjuvant chemotherapy.

### D. Discussion

Management of patients with stage II colon cancer remains a challenge for treating oncologists. Although the adjuvant concept is well established in stage III colon cancer based on survival benefit, the effect of fluoropyrimidine-based chemotherapy for patients with stage II disease is limited. Large pooled analyses have not shown a survival increase in the stage II colon cancer population (Figueredo et al. 2004; Mamounas et al. 1999; Quasar Collaborative Group et al. 2007). Limiting the patient population to those with stage II colon cancer, as in the QUASAR study—the largest randomized clinical trial conducted in this population to date—did not show a survival benefit in this subset of patients (Figueredo et al. 2004; Mamounas et al. 1999; Quasar Collaborative Group et al. 2007).

New molecular biomarkers are needed to complement clinicopathological features in order to further distinguish stage II colon cancers. MMR status is an essential component for additional stratification of patients with stage II colon cancer, who represent approximately 25% of all colon cancer cases (Siegel et al 2014). Patients with stage II disease and a dMMR status have a significantly better prognosis compared with patients who have dMMR or pMMR tumors and they do not seem to benefit from adjuvant 5-fluoropyrimidine-based chemotherapy, as described in the most recent NCCN guidelines (National Comprehensive Cancer Network, 2016).

In the NCCN guidelines, patients with pMMR and T3 tumors without clinical risk factors have, after initial surgery, four different adjuvant treatment options; patients with T3 at high risk for systemic recurrence or T4 tumors have seven different options ranging from observation to different choices of adjuvant therapies. Consequently, the treating oncologist still faces a dilemma as to how to manage patients with pMMR stage II colon cancer.

The aim of this study was to investigate if the MACC1 gene could be a useful additional biomarker—in conjunction with MMR status—to further stratify patients with pMMR stage II colon cancer to identify patients at high risk of recurrence who would potentially benefit from adjuvant chemotherapy or conversely to identify patients who perform well biologically without adjuvant chemotherapy. MACC1 was selected as a proven, tumor stage-independent, prognostic colon cancer biomarker centrally involved in colon cancer tumor progression and with the ability to regulate genes involved in metastasis, e.g. c-Met (Stein et al., Nat Med 2009; Arlt & Stein 2009; Stein et al. Cell Cycle 2009; Wang et al. 2015; Wu et al. 2015; Schmid et al. Oncogene 2015).

To the best of our knowledge this is the first time that a combination of MMR and MACC1 has been analyzed in colon cancer with a particular focus on stage II disease. To investigate the role of MMR and MACC1, tissue samples from a cohort of 189 patients with stage II colon cancer, enriched for disease recurrence, were tested for MMR and MACC1 status (BIOGRID 1). As expected, MACC1 and MMR were both prognostic biomarkers with the ability to separate patients based on their RFS. qRT-PCR and immunohistochemistry methods were used for MACC1 mRNA or protein detection for the BIOGRID 1 cohort, both methodologies having previously been extensively tested and validated in two previous colon cancer tissue cohorts (Charité 1 and 2).

Combining MACC1 as a stratification marker for patients with pMMR tumors gave prognostic separation for RFS between the pMMR/MACC1 low and pMMR/MACC1 high groups for patients with T3 or T3/T4 disease, as well as in patients with or without adjuvant chemotherapy. In particular, 7% and 13% of patients were identified as pMMR/MACC1 low using immunohistochemistry and qRT-PCR, respectively. These patients had a similar prognosis with regard to RFS as patients with dMMR tumors. To date, none of the pMMR/MACC1 low (immunohistochemistry) patients has relapsed.

Next, a validation cohort of 306 patients was analyzed (BIOGRID 2). This time—and in contrast to BIOGRID 1—the cohort was not enriched for patients with tumor recurrence and only MACC1 immunohistochemistry was used, which is simple to perform, in conjunction with MMR status. The prognostic separation of pMMR/MACC1 low and pMMR/MACC1 high was confirmed for patients with T3 or T3/T4 staged tumors as well as for chemotherapy-naïve and -treated patients. In the BIOGRID 2 cohort, 5% of patients had a pMMR/MACC1 low expression profile, and their clinical outcome was similar to that of patients with the favorable pMMR status. Similar to BIOGRID 1, no tumor recurrence was observed in the pMMR/MACC1 low group, with a median follow-up in excess of 5 years.

As the Sample size was not statistically derived for Biogrid 1 or 2 no estimate of effect size was available prior to this study, cohorts B1 and B2 were pooled to obtain the most precise estimate of MACC1 informatively. Pooling of patients in Biogrid 1 and 2 significantly separates chemo naïve pMMR patients using MACC1 IHC. 6% of patients with pMMR/MACC1 low did not have a recurrence of disease demonstrating a significant better RFS of 100% as compared to pMMR/MACC1 high patients suggesting a similar favorable outcome as those patients that are pMMR.

The results of the two BIOGRID cohorts separate and pooled, which comprise 495 patients with stage II colon cancer, imply that: (1) there is a high degree of overlap (>90%) between patients with pMMR and MACC1 high tumors and (2) MACC1 can be a useful biomarker to identify the subset of 5-7% (immunohistochemistry) and 13% (qRT-PCR) of patients with pMMR/MACC1 low disease. Despite the small percentage range, the identification of this patient segment seems clinically relevant as it might add to the 15% of patients with dMMR who have a more favorable prognosis than those with pMMR and who might not benefit from fluoropyrimidine-based adjuvant chemotherapy.

Thus, based on the current analysis, we hypothesize that patients with pMMR/MACC1 low stage II colon cancer might not benefit from additional adjuvant chemotherapy based on their favorable prognosis, with a RFS of 100% after 5 years.

In summary, we have provided evidence that the combination of MMR status and simple immunohistochemistry testing of MACC1 protein levels has utility to identify a distinct stage II colon cancer population that is pMMR/MACC1 low with a favorable prognosis and an RFS similar to that observed in patients with colon cancer of dMMR status. This finding warrants further investigation of the use of MACC1 expression for stratification of patients with stage II pMMR colon cancer in prospective clinical trials.

While the invention has been described in detail with reference to specific examples, it will be apparent to one skilled in the art that various modifications can be made within the scope of this invention. Thus the scope of the invention should not be limited by the examples described herein. All patents, publications, websites, Genbank (or other database) entries disclosed herein are incorporated by reference in their entireties.

### REFERENCES

Siegel R, Desantis C, Jemal A: Colorectal cancer statistics, 2014. CA Cancer J Clin 64:104-117, 2014.
Edge M, Byrd D, Compton C, et al: AJCC Cancer Staging Manual. 7th ed. New York, N.Y.: Springer, 2010.
National Comprehensive Cancer Network. Clinical Practice Guidelines in Oncology (NCCN Guidelines®): Colon Cancer. Version 2.2016. https://www.nccn.org/profes sionals/physician_gls/pdf/colon.pdf
Figueredo A, Charette M L, Maroun J, et al: Adjuvant therapy for stage II colon cancer: a systematic review from the Cancer Care Ontario Program in evidence-based care's gastrointestinal cancer disease site group. J Clin Oncol 22:3395-3407, 2004
Mamounas E, Wieand S, Wolmark N, et al: Comparative efficacy of adjuvant chemotherapy in patients with Dukes' B versus Dukes' C colon cancer: results from four national surgical adjuvant breast and bowel project adjuvant studies (C-01, C-02, C-03, and C-04). J Clin Oncol 17:1349-1355, 1999
Quasar Collaborative Group, Gray R, Barnwell J, et al. Adjuvant chemotherapy versus observation in patients with colorectal cancer: a randomised study. Lancet 370:2020-2029, 2007
Tournigand C, Andre T, Bonnetain F, et al: Adjuvant therapy with fluorouracil and oxaliplatin in stage II and elderly patients (between ages 70 and 75 years) with colon cancer: subgroup analyses of the multicenter international study of oxaliplatin, fluorouracil, and leucovorin in the adjuvant treatment of colon cancer trial. J Clin Oncol 30:3353-3360, 2012
Shi Q, Andre T, Grothey A, et al: Comparison of outcomes after fluorouracil-based adjuvant therapy for stages II and III colon cancer between 1978 to 1995 and 1996 to 2007: evidence of stage migration from the ACCENT database. J Clin Oncol 31:3656-3663, 2013
Sargent D J, Marsoni S, Monges G, et al: Defective mismatch repair as a predictive marker for lack of efficacy of fluorouracil-based adjuvant therapy in colon cancer. J Clin Oncol 28:3219-3226, 2010
Ribic C M, Sargent D J, Moore M J, et al: Tumor microsatellite-instability status as a predictor of benefit from fluorouracil-based adjuvant chemotherapy for colon cancer. N Engl J Med 349:247-257, 2003
Popat S, Hubner R, Houlston R S: Systematic review of microsatellite instability and colorectal cancer prognosis. J Clin Oncol 23:609-618, 2005
Stein U, Walther W, Arlt F, et al: MACC1, a newly identified key regulator of HGF-MET signaling, predicts colon cancer metastasis. Nat Med 15:59-67, 2009
Arlt F, Stein U: Colon cancer metastasis: MACC1 and Met as metastatic pacemakers. Int J Biochem Cell Biol 41:2356-2359, 2009
Stein U, Smith J, Walther W, et al: MACC1 controls Met: what a difference an Sp1 site makes. Cell Cycle 8:2467-2469, 2009
Pichorner A, Sack U, Kobelt D, et al: In vivo imaging of colorectal cancer growth and metastasis by targeting MACC1 with shRNA in xenografted mice. Clin Exp Metastasis 29:573-583, 2012
Schmid F, Wang Q, Huska M R, et al: SPON2, a newly identified target gene of MACC1, drives colorectal cancer metastasis in mice and is prognostic for colorectal cancer patient survival. Oncogene 2015 Dec. 21. doi: 10.1038/onc.2015.451. [Epub ahead of print]
Lemos C, Hardt M S, Juneja M, et al: MACC1 induces tumor progression in transgenic mice and colorectal cancer patients via increased pluripotency markers Nanog and Oct4. Clin Cancer Res 22:2812-2824, 2016
Nitsche U, Rosenberg R, Balmert A, et al: Integrative marker analysis allows risk assessment for metastasis in stage II colon cancer. Ann Surg 256:763-771, 2012
Stein U, Burock S, Herrmann P, et al: Circulating MACC1 transcripts in colorectal cancer patient plasma predict metastasis and prognosis. PLoS One 7:e49249, 2012
Stein U. MACC1—a novel target for solid cancers. Expert Opin Ther Targets 17:1039-52, 2013
Ilm K, Kemmner W, Osterland M, et al: High MACC1 expression in combination with mutated KRAS G13 indicates poor survival of colorectal cancer patients. Mol Cancer 14:38, 2015
Koelzer V H, Herrmann P, Zlobec I, et al: Heterogeneity analysis of Metastasis Associated in Colon Cancer 1 (MACC1) for survival prognosis of colorectal cancer patients: a retrospective cohort study. BMC Cancer 15:160, 2015
Wang G, Fu Z, Li D. MACC1 overexpression and survival in solid tumors: a meta-analysis. Tumour Biol 36:1055-1065, 2015
Wu Z, Zhou R, Su Y, et al: Prognostic value of MACC1 in digestive system neoplasms: a systematic review and meta-analysis. Biomed Res Int 2015:252043, 2015
Sun D W, Zhang Y Y, Qi Y, et al: Prognostic and clinicopathological significance of MACC1 expression in hepatocellular carcinoma patients: a meta-analysis. Int J Clin Exp Med 8:4769-4777, 2015
Lederer A, Herrmann P, Seehofer P, et al: MACC1 is an independent prognostic biomarker for survival in Klatskin tumor patients. Hepatology 62:841-850, 2015
Bacher J W, Flanagan L A, Smalley R L, et al: Development of a fluorescent multiplex assay for detection of MSI-high tumors. Dis Markers 20:237-250, 2004
Gavin P G, Colangelo L H, Fumagalli D, et al: Mutation profiling and microsatellite instability in stage II and III colon cancer: an assessment of their prognostic and oxaliplatin predictive value. Clin Cancer Res 18:6531-6541, 2012
Wirtzfeld D A, Mikula L, Gryfe R, et al: Concordance with clinical practice guidelines for adjuvant chemotherapy in patients with stage I-III colon cancer: experience in 2 Canadian provinces. Can J Surg 52:92-97, 2009
Kopetz S, Tabernero J, Rosenberg R, et al: Genomic classifier ColoPrint predicts recurrence in stage II colorectal cancer patients more accurately than clinical factors. Oncologist 20:127-133, 2015
Sveen A, Nesbakken A, Agesen T H, et al: Anticipating the clinical use of prognostic gene expression-based tests for colon cancer stage II and III: is Godot finally arriving? Clin Cancer Res 19:6669-6677, 2013
Zlobec et al., Selecting immunohistochemical cut-off scores for novel biomarkers of progression and survival in colorectal cancer, J. Clin. Pathol. Vol. 60, pp. 1112-16 (December 2006)

What is claimed is:

1. A method of prognosing a subject having a mismatch repair-proficient (pMMR) colorectal cancer comprising detecting an expression level of a Metastasis Associated in Colon Cancer 1 (MACC1) gene product in a sample of the colorectal cancer and comparing the expression level to a reference expression level, wherein the subject has a unfavorable prognosis if the expression level of the MACC1 gene product exceeds the reference expression level, and wherein the subject has a favorable prognosis if the expression level of the MACC1 gene product falls below the reference expression level.

2. The method of claim 1, wherein the MACC1 gene product is MACC1 protein, optionally wherein the sample of the colorectal cancer is a cellular sample and the expression level of MACC1 protein is determined by:
contacting the cellular sample with a MACC1-biomarker-specific agent;
depositing a stain in proximity to the MACC1-biomarker-specific agent; and
detecting the stain microscopically,
further optionally wherein the expression level of MACC1 protein is a staining intensity, a percentage of stained tumor cells, or a combination of staining intensity and a percentage of stained tumor cells, optionally a percent of viable stained tumor cells, further optionally wherein the reference expression level is 75% of tumor cells demonstrating unequivocal cytoplasmic staining.

3. The method of claim 1, wherein the MACC1 gene product is MACC1 mRNA, optionally wherein the expression level of MACC1 mRNA is determined by reverse transcription and quantitative PCR (qRT-PCR) and/or optionally wherein the reference expression level is determined by receiver operating characteristic (ROC) analysis.

4. The method of claim 1, wherein the colorectal cancer is stage II colon cancer.

5. The method of claim 1, wherein the method is a method of identifying the subject having a mismatch repair-proficient (pMMR) colorectal cancer likely to benefit from a chemotherapy, wherein the subject is likely to benefit from chemotherapy if the expression level of the MACC1 gene product exceeds the reference expression level, and wherein the subject is unlikely to benefit from chemotherapy if the expression level of the MACC1 gene product falls below the reference expression level, optionally wherein the chemotherapy comprises a fluoropyrimidine-based chemotherapy and optionally further comprises leucovorin, further optionally wherein the fluoropyrimidine-based chemotherapy is selected from the group consisting of capecitabine, floxuridine, fluorouracil (5-FU), and combinations of capecitabine, floxuridine, and 5-FU.

6. A method of treating a subject suffering from a mismatch repair-proficient (pMMR) colorectal cancer, wherein an expression level of a Metastasis Associated in Colon Cancer 1 (MACC1) gene product has been detected in a subject's sample of the colorectal cancer and the expression level of the MACC1 gene product has been compared to a reference expression level, said method comprising:
administering a chemotherapeutic to the subject if the expression level of the MACC1 gene product exceeds the reference expression level, and administering a treatment course that does not include the chemotherapeutic or no treatment if the expression level of the MACC1 gene product falls below the reference expression level.

7. The method of claim 6, wherein:
a) the MACC1 gene product is MACC1 protein, optionally wherein the sample of the colorectal cancer is a cellular sample and the expression level of MACC1 protein is determined by:
contacting the cellular sample with a MACC1-biomarker-specific agent;
depositing a stain in proximity to the MACC1-biomarker-specific agent; and
detecting the stain microscopically,
further optionally wherein the expression level of MACC1 protein is a staining intensity, a percentage of stained tumor cells, or a combination of staining intensity and a percentage of stained tumor cells, optionally a percent of viable stained tumor cells, further optionally wherein the reference expression level is 75% of tumor cells demonstrating unequivocal cytoplasmic staining;
b) the MACC1 gene product is MACC1 mRNA, optionally wherein the expression level of MACC1 mRNA is determined by reverse transcription and quantitative PCR (qRT-PCR) and/or optionally wherein the reference expression level is determined by receiver operating characteristic (ROC) analysis;
c) the colorectal cancer is stage II colon cancer; or
d) the chemotherapeutic comprises a fluoropyrimidine-based chemotherapeutic and optionally further comprises leucovorin, optionally wherein the fluoropyrimidine-based chemotherapeutic is selected from the group consisting of capecitabine, floxuridine, fluorouracil (5-FU), and combinations of capecitabine, floxuridine, and 5-FU.

8. A kit for detection of a mismatch repair-proficient (pMMR) colorectal cancer likely to progress and/or respond to chemotherapy, said kit comprising:
a set of MMR-associated biomarker-specific agents, and a set of detection reagents suitable for detecting binding of the MMR-associated biomarker-specific agents to a sample of the colorectal cancer; and
one or more MACC1 gene product-biomarker-specific agents, and a set of detection reagents for detecting binding of the MACC1 gene product-biomarker-specific agent to a sample of the colorectal cancer,
optionally wherein the set of MMR-associated biomarker-specific agents includes an MLH1 gene product-biomarker-specific agent, an MSH2 gene product-biomarker-specific agent, an MSH6 gene product-biomarker-specific agent, and a PMS2 gene product-biomarker-specific agent,
further optionally wherein one or more of the MLH1 gene product, the MSH2 gene product, the MSH6 gene product, and the PMS2 gene product are proteins, even further optionally wherein each of the MLH1 gene product-biomarker-specific agent, the MSH2 gene product-biomarker-specific agent, the MSH6 gene product-biomarker-specific agent, and the PMS2 gene product-biomarker-specific agent is a monoclonal antibody, an antigen binding fragment of a monoclonal antibody, or an engineered specific binding structure; or
further optionally wherein
the set of MMR-associated biomarker-specific agents includes an MLH1-specific primer set, a MSH2-specific primer set, a MSH6-specific primer set, and a PMS2-specific primer set; and
the set of detection reagents suitable for detecting binding of the MMR-associated biomarker-specific agents to the sample of the colorectal cancer comprises:
reagents sufficient for performing reverse transcription; and
reagents sufficient for a polymerase chain reaction (PCR), even further optionally wherein the PCR is a quantitative PCR and the set of detection reagents suitable for detecting binding of the MMR-associated biomarker-specific agents to the sample of the colorectal cancer comprises further comprises a set of detection probes for performing the quantitative PCR.

9. The kit of claim 8, wherein the MACC1 gene product is a protein and the MACC1 gene product-biomarker-specific agent is a monoclonal antibody, an antigen binding fragment of a monoclonal antibody, or an engineered specific binding structure, optionally
a) wherein the sample of the colorectal cancer is a tissue section and the set of detection reagents for detecting binding of the MACC1 gene product-biomarker-specific agent comprises reagents sufficient for brightfield detection of the MACC1 gene product-biomarker-specific agent, or
b) wherein the sample of the colorectal cancer is a section of a tissue sample and the set of detection reagents for detecting binding of the MACC1 gene product-biomarker-specific agent comprises reagents sufficient for darkfield detection of the MACC1 gene product-biomarker-specific agent or
c) wherein the MACC1 gene product is MACC1 mRNA,
(i) further optionally wherein:
the MACC1 gene product-biomarker-specific agent includes a MACC1-specific primer set; and
the set of detection reagents suitable for detecting binding of the MACC1 gene product-biomarker-specific agent to the sample of the colorectal cancer comprises:
reagents sufficient for performing reverse transcription; and
reagents sufficient for a polymerase chain reaction (PCR), even further optionally wherein the PCR is a quantitative PCR and the set of detection reagents suitable for detecting binding of the MACC1 gene product-biomarker-specific agents to the sample of the colorectal cancer further comprises a set of detection probes for performing the quantitative PCR,
optionally wherein the MACC1-specific primer set comprises a forward primer comprising SEQ ID NO:1 and a reverse primer comprising SEQ ID NO:2 and/or
optionally wherein the set of detection probes for performing the quantitative PCR comprises:
(a) a first nucleic acid probe comprising a first nucleotide sequence complementary to the portion of a MACC1 cDNA that is amplified in the quantitative PCR, wherein the first nucleic acid probe is labeled with a first fluorophore at a 3′ end of the first nucleotide sequence, the first fluorophore being capable of emitting fluorescent light at a first wavelength; and
(b) a second nucleic acid probe comprising a second nucleotide sequence complementary to the portion of the MACC1 cDNA that is amplified, wherein the second nucleic acid probe is labeled with a second fluorophore at a 5′ end of the second nucleotide sequence, the second fluorophore being capable of emitting fluorescent light when excited by fluorescent light at the first wavelength; and
wherein the first nucleic acid probe and the second nucleic acid probe are close enough when hybridized to the portion of the MACC1 cDNA that is amplified that emission of fluorescent light from the first fluorophore excites the second fluorophore via fluorescence resonance energy transfer (FRET), and wherein detection comprises measuring a quantity of fluorescent light emitted by the second fluorophore,
optionally wherein the first sequence comprises SEQ ID NO:3 and the second sequence comprises SEQ ID NO:4, and/or
optionally wherein the first fluorophore is a fluorescein and
the second fluorophore emits a red fluorescence, or even further optionally wherein the set of detection probes for performing the quantitative PCR comprises a nucleic acid probe complementary to a portion of the MACC1 cDNA that is amplified, wherein the nucleic acid probe comprises a nucleotide sequence hybridizable to the MACC1 cDNA during amplification, a quencher at a 5′ end of the probe, and a fluorophore at a 3′ end of the probe, such that the quencher prevents detection of fluorescent light emitted from the fluorophore when the nucleic acid probe is intact, and wherein the cDNA is amplified by a polymerase with 5′ exonuclease activity, such that amplification releases the quencher from the nucleic acid probe, thereby allowing detection of the fluorophore,
optionally wherein the MACC1-specific primer set comprises a forward primer comprising SEQ ID NO:5 and a reverse primer comprising SEQ ID NO:6,
optionally wherein the nucleic acid probe comprises SEQ ID NO:7, the 5′ quencher is FAM, and the 3′ fluorophore is BHQ2.
(ii) further optionally wherein the kit further comprises a set of reagents for determining a quantity of a housekeeping cDNA, the set of reagents for determining a quantity of a housekeeping cDNA comprising a primer set specific for a housekeeping cDNA and reagents sufficient for amplifying at least a portion of the housekeeping cDNA,
optionally wherein the housekeeping cDNA is an HPRT cDNA or a G6PD cDNA,
optionally wherein the primer set specific for HPRT cDNA comprises a forward primer comprising SEQ ID NO:8 and a reverse primer comprising SEQ ID NO:9; or the primer pair specific for G6PD cDNA comprises a forward primer comprising SEQ ID NO:11 and a reverse primer comprising SEQ ID NO:12, and/or
further optionally wherein the kit further comprising a nucleic acid probe complementary to a portion of the housekeeping cDNA that is amplified, wherein the nucleic acid probe comprises a nucleotide sequence hybridizable to the housekeeping cDNA during amplification, a quencher at a 5′ end of the probe, and a fluorophore at a 3′ end of the probe, such that the quencher prevents detection of fluorescent light emitted from the fluorophore when the nucleic acid probe is intact, and wherein the cDNA is amplified by a polymerase with 5′ exonuclease activity, such that amplification releases the quencher from the nucleic acid probe, thereby allowing detection of the fluorophore.

10. The method of claim 1, comprising:
(a) providing a sample of a colorectal cancer obtained from the subject;
(b) contacting the sample with:
(b1) an antibody specific for MLH1;
(b2) an antibody specific for MSH2;
(b3) an antibody specific for MSH6;
(b4) an antibody specific for PMS2;
(b5) an antibody specific for MACC1; and
(b6) detection reagents sufficient for visualizing binding of each of (b1)-(b6) via brightfield or darkfield microscopy; and
(c) detecting via brightfield or darkfield microscopy the presence or absence of binding of each of (b1)-(b4) to the sample, and the quantity of binding of (b5) to the sample, wherein:
(c1) the subject has a favorable prognosis if:
(c1a) binding of at least one of (b1)-(b4) is absent, or
(c1b) binding of each of (b1)-(b4) is present and the number of viable tumor cells having (b5) bound thereto is below a threshold level; and
(c2) the subject has an unfavorable prognosis if binding of each of (b1)-(b4) is present and the number of viable tumor cells having (b5) bound thereto is above a threshold level
or
(c′) detecting via brightfield or darkfield microscopy the presence or absence of binding of each of (b1)-(b4) to the sample, and the quantity of binding of (b5) to the sample; and
(d′) identifying the subject as to be treated by chemotherapy, wherein:
(d1) the treatment does not comprise a chemotherapy if:
(d1a) binding of at least one of (b1)-(b4) is absent, or
(d′1b) binding of each of (b1)-(b4) is present and the number of viable tumor cells having (b5) bound thereto is below a threshold level; and
(d′2) the treatment comprises a chemotherapy if binding of each of (b1)-(b4) is present and the number of viable tumor cells having (b5) bound thereto is above a threshold level.

11. The method of claim 10, wherein the sample is a tissue sample and (i) each of (b1)-(b5) is contacted with a separate section of the tissue sample or (ii) at least two of (b1)-(b5) is contacted with the same section of the tissue sample.

12. The method of claim 1, comprising:
(a) providing a sample of a colorectal cancer obtained from the subject;
(b) generating a composition comprising cDNA generated from mRNA of the sample by contacting a first portion of the sample with reagents sufficient for performing reverse transcription;
(c) contacting the composition comprising cDNA with:
(c1) a primer pair specific for MLH1 cDNA;
(c2) a primer pair specific for MSH2 cDNA;
(c3) a primer pair specific for MSH6 cDNA;
(c4) a primer pair specific for PMS2 cDNA;
(c5) a primer pair specific for MACC1 cDNA; and
(c6) reagents sufficient for amplifying at least a portion of the MLH1 cDNA, MSH2 cDNA, MSH6 cDNA, PMS2 cDNA, and MACC1 cDNA if present;
(d) detecting the presence or absence of MLH1 cDNA, MSH2 cDNA, MSH6 cDNA, and PMS2 cDNA, and the quantity of MACC1 cDNA, wherein:
(d1) the subject has a favorable prognosis if:
(d1a) at least one of MLH1 cDNA, MSH2 cDNA, MSH6 cDNA, PMS2 cDNA is absent, or
(d1b) each of MLH1 cDNA, MSH2 cDNA, MSH6 cDNA, PMS2 cDNA is present and the quantity of MACC1 cDNA is below a threshold level; and
(d2) the subject has a unfavorable prognosis if each of MLH1 cDNA, MSH2 cDNA, MSH6 cDNA, PMS2 cDNA is present and the quantity of MACC1 cDNA is above a threshold level,
or
(d′) detecting the presence or absence of MLH1 cDNA, MSH2 cDNA, MSH6 cDNA, and PMS2 cDNA, and the quantity of MACC1 cDNA, and
(e′) identifying the subject as to be treated by chemotherapy, wherein:
(e1) the treatment does not comprise a chemotherapy if:
(e′1a) at least one of MLH1 cDNA, MSH2 cDNA, MSH6 cDNA, PMS2 cDNA is absent, or
(e′1b) each of MLH1 cDNA, MSH2 cDNA, MSH6 cDNA, PMS2 cDNA is present and the quantity of MACC1 cDNA is below a threshold level; and
(e′2) the treatment comprises a chemotherapy if each of MLH1 cDNA, MSH2 cDNA, MSH6 cDNA, PMS2 cDNA is present and the quantity of MACC1 cDNA is above a threshold level.

13. The method of claim 1, comprising:
(a) providing a sample of a colorectal cancer obtained from the subject;
(b) generating a composition comprising cDNA generated from mRNA of the sample by contacting a first portion of the sample with reagents sufficient for performing reverse transcription;
(c) contacting the composition comprising cDNA with:
(c1) a primer pair specific for MLH1 cDNA;
(c2) a primer pair specific for MSH2 cDNA;
(c3) a primer pair specific for MSH6 cDNA;
(c4) a primer pair specific for PMS2 cDNA; and
(c5) reagents sufficient for amplifying at least a portion of the MLH1 cDNA, MSH2 cDNA, MSH6 cDNA, and PMS2 cDNA if present;
(d) contacting a second portion of the sample with:
(d1) an antibody specific for MACC1; and
(d2) detection reagents sufficient for visualizing binding of the antibody specific for MACC1 via brightfield or darkfield microscopy;
(f) detecting:
(f1) the presence or absence of MLH1 cDNA, MSH2 cDNA, MSH6 cDNA, and PMS2 cDNA; and
(f2) the quantity of binding of the antibody specific for MACC1 to the second portion of the sample via brightfield or darkfield microscopy; wherein:
(f1) the subject has a favorable prognosis if:
(f1a) at least one of MLH1 cDNA, MSH2 cDNA, MSH6 cDNA, PMS2 cDNA is absent, or
(f1b) each of MLH1 cDNA, MSH2 cDNA, MSH6 cDNA, PMS2 cDNA is present and the quantity of binding of MACC1 to the second portion of the sample is below a threshold level; and
(f2) the subject has a unfavorable prognosis if each of MLH1 cDNA, MSH2 cDNA, MSH6 cDNA, PMS2 cDNA is present and the quantity of binding of MACC1 antibody to the second portion of the sample is above a threshold level.
or
(f′) detecting:
(f′1) the presence or absence of MLH1 cDNA, MSH2 cDNA, MSH6 cDNA, and PMS2 cDNA; and
(f′2) the quantity of binding of the antibody specific for MACC1 to the second portion of the sample via brightfield or darkfield microscopy;
(g′) identifying the subject as to be treated by chemotherapy, wherein:
(g′1) the treatment does not comprise a chemotherapy if:
(g′1a) at least one of MLH1 cDNA, MSH2 cDNA, MSH6 cDNA, PMS2 cDNA is absent, or
(g′1b) each of MLH1 cDNA, MSH2 cDNA, MSH6 cDNA, PMS2 cDNA is present and the quantity of binding of MACC1 antibody is below a threshold level; and
(g′2) the treatment comprises a chemotherapy if each of MLH1 cDNA, MSH2 cDNA, MSH6 cDNA, PMS2 cDNA is present and the quantity of the quantity of binding of MACC1 antibody is above a threshold level.

14. The method of claim 1, comprising:
(a) providing a sample of a colorectal cancer obtained from the subject;
(b) contacting a first portion of the sample with:
(b1) an antibody specific for MLH1;
(b2) an antibody specific for MSH2;
(b3) an antibody specific for MSH6;
(b4) an antibody specific for PMS2; and
(b5) detection reagents sufficient for visualizing binding of each of (b1)-(b5) via brightfield or darkfield microscopy
(c) generating a composition comprising cDNA generated from mRNA of the sample by contacting a second portion of the sample with reagents sufficient for performing reverse transcription;
(d) contacting the composition comprising cDNA with:
(d1) a primer pair specific for MACC1 cDNA; and
(d2) reagents sufficient for amplifying at least a portion of the MACC1 cDNA if present;
(e) detecting:
(e1) the presence or absence of binding of each of (b1)-(b4) to the first portion of the sample via brightfield or darkfield microscopy, and
(e2) the quantity of MACC1 cDNA in the second portion of the sample, wherein:
(e1) the subject has a favorable prognosis if:
(e1a) binding of at least one of (b1)-(b4) is absent, or
(e1b) binding of each of (b1)-(b4) is present and the quantity of MACC1 cDNA is below a threshold level; and
(e2) the subject has a unfavorable prognosis if binding of each of (b1)-(b4) is present and the quantity of MACC1 cDNA is above a threshold level.
or
(e′) detecting:
(e′ 1) the presence or absence of binding of each of (b1)-(b4) to the first portion of the sample via brightfield or darkfield microscopy, and
(e′2) the quantity of MACC1 cDNA in the second portion of the sample
(g′) identifying the subject as to be treated by chemotherapy, wherein:
(g′1) the treatment does not comprise a chemotherapy if:
(g1a) binding of at least one of each of (b1)-(b4) to the first portion of the sample is absent, or
(g1b) binding of each of (b1)-(b4) to the first portion of the sample is present and the quantity of MACC1 cDNA is below a threshold level; and
(g′2) the treatment comprises a chemotherapy if binding of each of (b1)-(b4) to the first portion of the sample is present and the quantity of MACC1 cDNA is above a threshold level,
optionally wherein the first portion of the sample is a tissue sample and each of (b1)-(b4) is contacted with a separate section of the tissue sample or wherein the first portion of the sample is a tissue sample and at least two of (b1)-(b5) is contacted with the same section of the tissue sample.

15. The method of claim 12,
a) wherein the chemotherapy comprises a fluoropyrimidine-based chemotherapy and optionally further comprises leucovorin, optionally wherein the fluoropyrimidine-based chemotherapy is selected from the group consisting of capecitabine, floxuridine, fluorouracil (5-FU), and combinations of capecitabine, floxuridine, and 5-FU,
b) wherein the primer pair specific for MACC1 cDNA includes a forward primer comprising SEQ ID NO:1 and a reverse primer comprising SEQ ID NO:2; and/or
c) wherein the quantity of MACC1 cDNA is detected using:
(a) a first nucleic acid probe comprising a first nucleotide sequence complementary to the portion of the MACC1 cDNA that is amplified, wherein the first nucleic acid probe is labeled with a first fluorophore at a 3′ end of the first nucleotide sequence, the first fluorophore being capable of emitting fluorescent light at a first wavelength; and
(b) a second nucleic acid probe comprising a second nucleotide sequence complementary to the portion of the MACC1 cDNA that is amplified, wherein the second nucleic acid probe is labeled with a second fluorophore at a 5′ end of the second nucleotide sequence, the second fluorophore being capable of emitting fluorescent light when excited by fluorescent light at the first wavelength; and
wherein the first nucleic acid probe and the second nucleic acid probe are close enough when hybridized to the portion of the MACC1 cDNA that is amplified that emission of fluorescent light from the first fluorophore excites the second fluorophore via fluorescence resonance energy transfer (FRET), and wherein detection comprises measuring a quantity of fluorescent light emitted by the second fluorophore,
optionally wherein the first sequence comprises SEQ ID NO:3 and the second sequence comprises SEQ ID NO:4 and/or
optionally wherein the first fluorophore is a fluorescein and the second fluorophore emits a red fluorescence; and/or
d) wherein the quantity of the MACC1 cDNA is detected using a nucleic acid probe complementary to a portion of the MACC1 cDNA that is amplified, wherein the nucleic acid probe comprises a nucleotide sequence hybridizable to the MACC1 cDNA during amplification, a quencher at a 5′ end of the probe, and a fluorophore at a 3′ end of the probe, such that the quencher prevents detection of fluorescent light emitted from the fluorophore when the nucleic acid probe is intact, and wherein the cDNA is amplified by a polymerase with 5′ exonuclease activity, such that amplification releases the quencher from the nucleic acid probe, thereby allowing detection of the fluorophore,
optionally wherein the primer pair specific for MACC1 cDNA includes a forward primer comprising SEQ ID NO:5 and a reverse primer comprising SEQ ID NO:6,
further optionally wherein the nucleic acid probe comprises SEQ ID NO:7, the 5′ quencher is FAM, and the 3′ fluorophore is BHQ2; or
optionally wherein detecting the quantity of the MACC1 cDNA further comprises comparing the quantity of the MACC1 cDNA to a quantity of a housekeeping cDNA, the method further comprising:
(d3) contacting the composition comprising cDNA with a primer pair specific for a housekeeping cDNA and reagents sufficient for amplifying at least a portion of the housekeeping cDNA,
further optionally wherein the housekeeping cDNA is an HPRT cDNA or a G6PD cDNA, optionally wherein:
the primer pair specific for HPRT cDNA includes a forward primer comprising SEQ ID NO:8 and a reverse primer comprising SEQ ID NO:9; or
the primer pair specific for G6PD cDNA includes a forward primer comprising SEQ ID NO:11 and a reverse primer comprising SEQ ID NO:12,
or optionally wherein the quantity of the housekeeping cDNA is detected using a nucleic acid probe complementary to a portion of the housekeeping cDNA that is amplified, wherein the nucleic acid probe comprises a nucleotide sequence hybridizable to the housekeeping cDNA during amplification, a quencher at a 5′ end of the probe, and a fluorophore at a 3′ end of the probe, such that the quencher prevents detection of fluorescent light emitted from the fluorophore when the nucleic acid probe is intact, and wherein the cDNA is amplified by a polymerase with 5′ exonuclease activity, such that amplification releases the quencher from the nucleic acid probe, thereby allowing detection of the fluorophore; or
e) wherein the colorectal cancer is stage II colon cancer.


SEQUENCE LISTING (27 sequences)

SEQ ID NO: 1 - DNA, 21 bp, Artificial Sequence
  Nucleic acid primer
ttcttttgattcctccggtga

SEQ ID NO: 2 - DNA, 20 bp, Artificial Sequence
  Nucleic Acid Primer
actctgatgggcatgtgctg

SEQ ID NO: 3 - DNA, 32 bp, Artificial Sequence
  Nucleic acid probe
gcagacttcctcaagaaattctggaagatcta

SEQ ID NO: 4 - DNA, 32 bp, Artificial Sequence
  Nucleic acid probe
agtgtttcagaacttctggacattttagacga

SEQ ID NO: 5 - DNA, 24 bp, Artificial Sequence
  Nucleic acid primer
attgacatggaagctggaaaactc

SEQ ID NO: 6 - DNA, 20 bp, Artificial Sequence
  Nucleic acid primer
cacgaagggtgaaagcatcc

SEQ ID NO: 7 - DNA, 37 bp, Artificial Sequence
  Nucleic acid probe
tacagaatgccaggacccagacttgcttcacaattgg

SEQ ID NO: 8 - DNA, 17 bp, Artificial Sequence
  Nucleic acid primer
tgctgtgtctggtggcc

SEQ ID NO: 9 - DNA, 23 bp, Artificial Sequence
  Nucleic acid primer
gcatttcaacaccttgaccttct

SEQ ID NO: 10 - DNA, 29 bp, Artificial Sequence
  Nucleic acid probe
agaagcccgcctccaccaactcagatgac

SEQ ID NO: 11 - DNA, 25 bp, Artificial Sequence
  Nucleic acid primer
gaccttgatttattttgcataccta

SEQ ID NO: 12 - DNA, 19 bp, Artificial Sequence
  Nucleic acid primer
gagcaagacgttcagtcct

SEQ ID NO: 13 - DNA, 30 bp, Artificial Sequence
  Nucleic acid probe
atgctgaggatttggaaagggtgtttattc

SEQ ID NO: 14 - protein, 852 aa, Homo sapiens
MLITERKHFRSGRIAQSMSEANLIDMEAGKLSKSCNITECQDPDLLHNWPDAFTLRGNNASKVANPFWNQLSASNPFLDDITQLRNNRKRNNISILKEDPFLFCREIENGNSFDSSGDELDVHQLLRQTSSRNSGRSKSVSELLDILDDTAHAHQSIHNSDQILLHDLEWLKNDREAYKMAWLSQRQLARSCLDLNTISQSPGWAQTQLAEVTIACKVNHQGGSVQLPESDITVHVPQGHVAVGEFQEVSLRAFLDPPHMLNHDLSCTVSPLLEIMLGNLNTMEALLLEMKIGAEVRKDPFSQVMTEMVCLHSLGKEGPFKVLSNCYIYKDTIQVKLIDLSQVMYLVVAAQAKALPSPAATIWDYIHKTTSIGIYGPKYIHPSFTVVLTVCGHNYMPGQLTISDIKKGGKNISPVVFQLWGKQSFLLDKPQDLSISIFSCDPDFEVKTEGERKEIKQKQLEAGEVVHQQFLFSLVEHREMHLFDFCVQVEPPNGEPVAQFSITTPDPTPNLKRLSNLPGYLQKKEEIKSAPLSPKILVKYPTFQDKTLNFSNYGVTLKAVLRQSKIDYFLEYFKGDTIALLGEGKVKAIGQSKVKEWYVGVLRGKIGLVHCKNVKVISKEQVMFMSDSVFTTRNLLEQIVLPLKKLTYIYSVVLTLVSEKVYDWKVLADVLGYSHLSLEDFDQIQADKESEKVSYVIKKLKEDCHTERNTRKFLYELIVALLKMDCQELVARLIQEAAVLTSAVKLGKGWRELAEKLVRLTKQQMEAYEIPHRGNTGDVAVEMMWKPAYDFLYTWSAHYGNNYRDVLQDLQSALDRMKNPVTKHWRELTGVLILVNSLEVLRVTAFSTSEEV

SEQ ID NO: 15 - protein, 756 aa, Homo sapiens
MSFVAGVIRRLDETVVNRIAAGEVIQRPANAIKEMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGSNSSRMYFTQTLLPGLAGPSGEMVKSTTSLTSSSTSGSSDKVYAHQMVRTDSREQKLDAFLQPLSKPLSSQPQAIVTEDKTDISSGRARQQDEEMLELPAPAEVAAKNQSLEGDTTKGTSEMSEKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVFERC

SEQ ID NO: 16 - protein, 934 aa, Homo sapiens [sequence omitted]

SEQ ID NO: 17 - protein, 1360 aa, Homo sapiens
MSRQSTLYSFFPKSPALSDANKASARASREGGRAAAAPGASPSPGGDAAWSEAGPGPRPLARSASPPKAKNLNGGLRRSVAPAAPTSCDFSPGDLVWAKMEGYPWWPCLVYNHPFDGTFIREKGKSVRVHVQFFDDSPTRGWVSKRLLKPYTGSKSKEAQKGGHFYSAKPEILRAMQRADEALNKDKIKRLELAVCDEPSEPEEEEEMEVGTTYVTDKSEEDNEIESEEEVQPKTQGSRRSSRQIKKRRVISDSESDIGGSDVEFKPDTKEEGSSDEISSGVGDSESEGLNSPVKVARKRKRMVTGNGSLKRKSSRKETPSATKQATSISSETKNTLRAFSAPQNSESQAHVSGGGDDSSRPTVWYHETLEWLKEEKRRDEHRRRPDHPDFDASTLYVPEDFLNSCTPGMRKWWQIKSQNFDLVICYKVGKFYELYHMDALIGVSELGLVFMKGNWAHSGFPEIAFGRYSDSLVQKGYKVARVEQTETPEMMEARCRKMAHISKYDRVVRREICRIITKGTQTYSVLEGDPSENYSKYLLSLKEKEEDSSGHTRAYGVCFVDTSLGKFFIGQFSDDRHCSRFRTLVAHYPPVQVLFEKGNLSKETKTILKSSLSCSLQEGLIPGSQFWDASKTLRTLLEEEYFREKLSDGIGVMLPQVLKGMTSESDSIGLTPGEKSELALSALGGCVFYLKKCLIDQELLSMANFEEYIPLDSDTVSTTRSGAIFTKAYQRMVLDAVTLNNLEIFLNGTNGSTEGTLLERVDTCHTPFGKRLLKQWLCAPLCNHYAINDRLDAIEDLMVVPDKISEVVELLKKLPDLERLLSKIHNVGSPLKSQNHPDSRAIMYEETTYSKKKIIDFLSALEGFKVMCKIIGIMEEVADGFKSKILKQVISLQTKNPEGRFPDLTVELNRWDTAFDHEKARKTGLITPKAGFDSDYDQALADIRENEQSLLEYLEKQRNRIGCRTIVYWGIGRNRYQLEIPENFTTRNLPEEYELKSTKKGCKRYWTKTIEKKLANLINAEERRDVSLKDCMRRLFYNFDKNYKDWQSAVECIAVLDVLLCLANYSRGGDGPMCRPVILLPEDTPPFLELKGSRHPCITKTFFGDDFIPNDILIGCEEEEQENGKAYCVLVTGPNMGGKSTLMRQAGLLAVMAQMGCYVPAEVCRLTPIDRVFTRLGASDRIMSGESTFFVELSETASILMHATAHSLVLVDELGRGTATFDGTAIANAVVKELAETIKCRTLFSTHYHSLVEDYSQNVAVRLGHMACMVENECEDPSQETITFLYKFIKGACPKSYGFNAARLANLPEEVIQKGHRKAREFEKMNQSLRLFREVCLASERSTVDAEAVHKLLTLIKEL

SEQ ID NO: 18 - DNA, 2559 bp, Homo sapiens
atgctaatcactgaaagaaaacattttcggtcaggaagaattgcacaaagtatgtctgaagcaaatttgattgacatggaagctggaaaactctcaaaaagttgcaatattacagaatgccaggacccagacttgcttcacaattggccggatgctttcacccttcgtggtaataatgcttccaaagttgcaaatccattctggaatcaactgtctgcttctaacccatttttggatgacataactcaactaagaaataacaggaagagaaataatatttccatcttaaaggaagatccttttcttttctgtagagaaatagaaaatggaaattcttttgattcctccggtgatgaacttgatgtgcatcagttacttaggcagacttcctcaagaaattctggaagatctaaaagtgtttcagaacttctggacattttagacgacacagcacatgcccatcagagtatacataactctgaccagatcctactacacgacttagagtggcttaaaaatgatcgggaggcttataaaatggcttggttaagtcaacgccagctggcccgctcctgccttgatttgaatacaattagtcagagccctggatgggcccagacacaacttgcggaggtcaccatagcttgcaaagtaaaccatcaaggagggtcagtacaattacctgaatcagacatcactgttcatgtgccccaaggtcatgtggctgtgggagaattccaagaggtgtctctaagggctttccttgatccgccacacatgcttaaccatgatctttcgtgcactgtgagcccgttgttggaaatcatgttaggcaacctcaatacaatggaagcccttttgctggagatgaaaattggggctgaagtaagaaaggatcctttcagccaagtcatgacagaaatggtgtgtttacacagcttgggtaaagaaggcccttttaaagttttaagcaactgctacatttataaagacaccatccaagtcaagctaatcgacttgagtcaggtaatgtatctagtggttgctgcacaagctaaagctcttccgtcaccagctgccaccatttgggattatatccacaaaaccacctcaattggaatttatggacccaaatatatccatcccagttttactgttgttttaacagtttgtggacacaattatatgccaggacagcttacaatttctgatattaagaagggtggaaaaaacatatctccagttgtgtttcagctctgggggaagcagtcatttttacttgacaagccacaagatttaagtatttctattttttcctgtgatcctgattttgaagtaaagacagaaggagaaaggaaagaaattaaacaaaagcagttggaagcaggtgaagtagttcatcaacaatttttattttctttagttgagcacagagagatgcacttgtttgatttttgtgttcaagtggagcctcccaatggtgaaccagttgcacagttctctatcactactcctgatccaaccccaaacctaaaaagactctcgaatctgccaggctatttgcagaagaaggaggaaatcaagtctgctcctttatcaccaaaaattcttgttaaatatcctacatttcaagataaaacattgaactttagcaactatggggtaaccctgaaggcagtgctaagacaaagcaagattgattacttccttgaatatttcaaaggggacacaatagctctcctcggggaaggtaaggtaaaagctattggtcagtccaaagtgaaagaatggtatgtaggagtcctcagaggtaagattggacttgtacactgcaaaaatgtcaaggtgatttcaaaggagcaagtaatgtttatgtcagatagtgtctttacaaccagaaatcttcttgaacagattgtcctgcctttaaaaaaattgacttatatctactcagttgtattaaccttggtgtcagaaaaagtttatgattggaaagttttagctgatgtcctgggttactcacatctgtccctggaagattttgatcaaattcaagcagacaaagaatcagagaaagtttcttatgttataaagaagttaaaggaagattgccacacagagagaaatacaaggaagtttctgtatgaacttattgtggctcttctgaaaatggattgccaagagttagtcgcacgtctcatccaagaagctgctgttctgacttcagctgtcaagcttggaaaaggctggagggaactagctgaaaagttagtacgactcacaaagcaacaaatggaggcatatgaaattcctcatcgaggaaacactggagatgttgctgttgagatgatgtggaaacctgcctatgattttctgtatacctggagtgctcactatggaaataactacagagatgtgttacaagaccttcagtcagctttggacagaatgaaaaaccctgtgactaaacactggagagaattaactggagttttaatactagtaaattctttggaggttttgagagtaactgcattctccacttctgaggaagtatag

SEQ ID NO: 19 - DNA, 1638 bp, Homo sapiens
atgggccggcggggctcagcccccggaaacggtcgtacacttcggggctgcgagcgcggagggcgacgacgacgaagcgcagacagcgtcatggcagagcaggtggccctgagccggacccaggtgtgcgggatcctgcgggaagagcttttccagggcgatgccttccatcagtcggatacacacatattcatcatcatgggtgcatcgggtgacctggccaagaagaagatctaccccaccatctggtggctgttccgggatggccttctgcccgaaaacaccttcatcgtgggctatgcccgttcccgcctcacagtggctgacatccgcaaacagagtgagcccttcttcaaggccaccccagaggagaagctcaagctggaggacttctttgcccgcaactcctatgtggctggccagtacgatgatgcagcctcctaccagcgcctcaacagccacatgaatgccctccacctggggtcacaggccaaccgcctcttctacctggccttgcccccgaccgtctacgaggccgtcaccaagaacattcacgagtcctgcatgagccagataggctggaaccgcatcatcgtggagaagcccttcgggagggacctgcagagctctgaccggctgtccaaccacatctcctccctgttccgtgaggaccagatctaccgcatcgaccactacctgggcaaggagatggtgcagaacctcatggtgctgagatttgccaacaggatcttcggccccatctggaaccgggacaacatcgcctgcgttatcctcaccttcaaggagccctttggcactgagggtcgcgggggctatttcgatgaatttgggatcatccgggacgtgatgcagaaccacctactgcagatgctgtgtctggtggccatggagaagcccgcctccaccaactcagatgacgtccgtgatgagaaggtcaaggtgttgaaatgcatctcagaggtgcaggccaacaatgtggtcctgggccagtacgtggggaaccccgatggagagggcgaggccaccaaagggtacctggacgaccccacggtgccccgcgggtccaccaccgccacttttgcagccgtcgtcctctatgtggagaatgagaggtgggatggggtgcccttcatcctgcgctgcggcaaggccctgaacgagcgcaaggccgaggtgaggctgcagttccatgatgtggccggcgacatcttccaccagcagtgcaagcgcaacgagctggtgatccgcgtgcagcccaacgaggccgtgtacaccaagatgatgaccaagaagccgggcatgttcttcaaccccgaggagtcggagctggacctgacctacggcaacagatacaagaacgtgaagctccctgacgcctatgagcgcctcatcctggacgtcttctgcgggagccagatgcacttcgtgcgcagcgacgagctccgtgaggcctggcgtattttcaccccactgctgcaccagattgagctggagaagcccaagcccatcccctatatttatggcagccgaggccccacggaggcagacgagctgatgaagagagtgggtttccagtatgagggcacctacaagtgggtgaacccccacaagctctga

SEQ ID NO: 20 - DNA, 1548 bp, Homo sapiens
atggcagagcaggtggccctgagccggacccaggtgtgcgggatcctgcgggaagagcttttccagggcgatgccttccatcagtcggatacacacatattcatcatcatgggtgcatcgggtgacctggccaagaagaagatctaccccaccatctggtggctgttccgggatggccttctgcccgaaaacaccttcatcgtgggctatgcccgttcccgcctcacagtggctgacatccgcaaacagagtgagcccttcttcaaggccaccccagaggagaagctcaagctggaggacttctttgcccgcaactcctatgtggctggccagtacgatgatgcagcctcctaccagcgcctcaacagccacatgaatgccctccacctggggtcacaggccaaccgcctcttctacctggccttgcccccgaccgtctacgaggccgtcaccaagaacattcacgagtcctgcatgagccagataggctggaaccgcatcatcgtggagaagcccttcgggagggacctgcagagctctgaccggctgtccaaccacatctcctccctgttccgtgaggaccagatctaccgcatcgaccactacctgggcaaggagatggtgcagaacctcatggtgctgagatttgccaacaggatcttcggccccatctggaaccgggacaacatcgcctgcgttatcctcaccttcaaggagccctttggcactgagggtcgcgggggctatttcgatgaatttgggatcatccgggacgtgatgcagaaccacctactgcagatgctgtgtctggtggccatggagaagcccgcctccaccaactcagatgacgtccgtgatgagaaggtcaaggtgttgaaatgcatctcagaggtgcaggccaacaatgtggtcctgggccagtacgtggggaaccccgatggagagggcgaggccaccaaagggtacctggacgaccccacggtgccccgcgggtccaccaccgccacttttgcagccgtcgtcctctatgtggagaatgagaggtgggatggggtgcccttcatcctgcgctgcggcaaggccctgaacgagcgcaaggccgaggtgaggctgcagttccatgatgtggccggcgacatcttccaccagcagtgcaagcgcaacgagctggtgatccgcgtgcagcccaacgaggccgtgtacaccaagatgatgaccaagaagccgggcatgttcttcaaccccgaggagtcggagctggacctgacctacggcaacagatacaagaacgtgaagctccctgacgcctatgagcgcctcatcctggacgtcttctgcgggagccagatgcacttcgtgcgcagcgacgagctccgtgaggcctggcgtattttcaccccactgctgcaccagattgagctggagaagcccaagcccatcccctatatttatggcagccgaggccccacggaggcagacgagctgatgaagagagtgggtttccagtatgagggcacctacaagtgggtgaacccccacaagctctga

SEQ ID NO: 21 - protein, 515 aa, Homo sapiens [sequence omitted]

SEQ ID NO: 22 - DNA, 657 bp, Homo sapiens
atggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaa

SEQ ID NO: 23 - protein, 218 aa, Homo sapiens
MATRSPGVVISDDEPGYDLDLFCIPNHYAEDLERVFIPHGLIMDRTERLARDVMKEMGGHHIVALCVLKGGYKFFADLLDYIKALNRNSDRSIPMTVDFIRLKSYCNDQSTGDIKVIGGDDLSTLTGKNVLIVEDIIDTGKTMQTLLSLVRQYNPKMVKVASLLVKRTPRSVGYKPDFVGFEIPDKFVVGYALDYNEYFRDLNHVCVISETGKAKYKA

SEQ ID NO: 24 - DNA, 2271 bp, Homo sapiens
atgtcgttcgtggcaggggttattcggcggctggacgagacagtggtgaaccgcatcgcggcgggggaagttatccagcggccagctaatgctatcaaagagatgattgagaactgtttagatgcaaaatccacaagtattcaagtgattgttaaagagggaggcctgaagttgattcagatccaagacaatggcaccgggatcaggaaagaagatctggatattgtatgtgaaaggttcactactagtaaactgcagtcctttgaggatttagccagtatttctacctatggctttcgaggtgaggctttggccagcataagccatgtggctcatgttactattacaacgaaaacagctgatggaaagtgtgcatacagagcaagttactcagatggaaaactgaaagcccctcctaaaccatgtgctggcaatcaagggacccagatcacggtggaggaccttttttacaacatagccacgaggagaaaagctttaaaaaatccaagtgaagaatatgggaaaattttggaagttgttggcaggtattcagtacacaatgcaggcattagtttctcagttaaaaaacaaggagagacagtagctgatgttaggacactacccaatgcctcaaccgtggacaatattcgctccatctttggaaatgctgttagtcgagaactgatagaaattggatgtgaggataaaaccctagccttcaaaatgaatggttacatatccaatgcaaactactcagtgaagaagtgcatcttcttactcttcatcaaccatcgtctggtagaatcaacttccttgagaaaagccatagaaacagtgtatgcagcctatttgcccaaaaacacacacccattcctgtacctcagtttagaaatcagtccccagaatgtggatgttaatgtgcaccccacaaagcatgaagttcacttcctgcacgaggagagcatcctggagcgggtgcagcagcacatcgagagcaagctcctgggctccaattcctccaggatgtacttcacccagactttgctaccaggacttgctggcccctctggggagatggttaaatccacaacaagtctgacctcgtcttctacttctggaagtagtgataaggtctatgcccaccagatggttcgtacagattcccgggaacagaagcttgatgcatttctgcagcctctgagcaaacccctgtccagtcagccccaggccattgtcacagaggataagacagatatttctagtggcagggctaggcagcaagatgaggagatgcttgaactcccagcccctgctgaagtggctgccaaaaatcagagcttggagggggatacaacaaaggggacttcagaaatgtcagagaagagaggacctacttccagcaaccccagaaagagacatcgggaagattctgatgtggaaatggtggaagatgattcccgaaaggaaatgactgcagcttgtaccccccggagaaggatcattaacctcactagtgttttgagtctccaggaagaaattaatgagcagggacatgaggttctccgggagatgttgcataaccactccttcgtgggctgtgtgaatcctcagtgggccttggcacagcatcaaaccaagttataccttctcaacaccaccaagcttagtgaagaactgttctaccagatactcatttatgattttgccaattttggtgttctcaggttatcggagccagcaccgctctttgaccttgccatgcttgccttagatagtccagagagtggctggacagaggaagatggtcccaaagaaggacttgctgaatacattgttgagtttctgaagaagaaggctgagatgcttgcagactatttctctttggaaattgatgaggaagggaacctgattggattaccccttctgattgacaactatgtgccccctttggagggactgcctatcttcattcttcgactagccactgaggtgaattgggacgaagaaaaggaatgttttgaaagcctcagtaaagaatgcgctatgttctattccatccggaagcagtacatatctgaggagtcgaccctctcaggccagcagagtgaagtgcctggctccattccaaactcctggaagtggactgtggaacacattgtctataaagccttgcgctcacacattctgcctcctaaacatttcacagaagatggaaatatcctgcagcttgctaacctgcctgatctatacaaagtctttgagaggtgttaa

SEQ ID NO: 25 - DNA, 2805 bp, Homo sapiens
atggcggtgcagccgaaggagacgctgcagttggagagcgcggccgaggtcggcttcgtgcgcttctttcagggcatgccggagaagccgaccaccacagtgcgccttttcgaccggggcgacttctatacggcgcacggcgaggacgcgctgctggccgcccgggaggtgttcaagacccagggggtgatcaagtacatggggccggcaggagcaaagaatctgcagagtgttgtgcttagtaaaatgaattttgaatcttttgtaaaagatcttcttctggttcgtcagtatagagttgaagtttataagaatagagctggaaataaggcatccaaggagaatgattggtatttggcatataaggcttctcctggcaatctctctcagtttgaagacattctctttggtaacaatgatatgtcagcttccattggtgttgtgggtgttaaaatgtccgcagttgatggccagagacaggttggagttgggtatgtggattccatacagaggaaactaggactgtgtgaattccctgataatgatcagttctccaatcttgaggctctcctcatccagattggaccaaaggaatgtgttttacccggaggagagactgctggagacatggggaaactgagacagataattcaaagaggaggaattctgatcacagaaagaaaaaaagctgacttttccacaaaagacatttatcaggacctcaaccggttgttgaaaggcaaaaagggagagcagatgaatagtgctgtattgccagaaatggagaatcaggttgcagtttcatcactgtctgcggtaatcaagtttttagaactcttatcagatgattccaactttggacagtttgaactgactacttttgacttcagccagtatatgaaattggatattgcagcagtcagagcccttaacctttttcagggttctgttgaagataccactggctctcagtctctggctgccttgctgaataagtgtaaaacccctcaaggacaaagacttgttaaccagtggattaagcagcctctcatggataagaacagaatagaggagagattgaatttagtggaagcttttgtagaagatgcagaattgaggcagactttacaagaagatttacttcgtcgattcccagatcttaaccgacttgccaagaagtttcaaagacaagcagcaaacttacaagattgttaccgactctatcagggtataaatcaactacctaatgttatacaggctctggaaaaacatgaaggaaaacaccagaaattattgttggcagtttttgtgactcctcttactgatcttcgttctgacttctccaagtttcaggaaatgatagaaacaactttagatatggatcaggtggaaaaccatgaattccttgtaaaaccttcatttgatcctaatctcagtgaattaagagaaataatgaatgacttggaaaagaagatgcagtcaacattaataagtgcagccagagatcttggcttggaccctggcaaacagattaaactggattccagtgcacagtttggatattactttcgtgtaacctgtaaggaagaaaaagtccttcgtaacaataaaaactttagtactgtagatatccagaagaatggtgttaaatttaccaacagcaaattgacttctttaaatgaagagtataccaaaaataaaacagaatatgaagaagcccaggatgccattgttaaagaaattgtcaatatttcttcaggctatgtagaaccaatgcagacactcaatgatgtgttagctcagctagatgctgttgtcagctttgctcacgtgtcaaatggagcacctgttccatatgtacgaccagccattttggagaaaggacaaggaagaattatattaaaagcatccaggcatgcttgtgttgaagttcaagatgaaattgcatttattcctaatgacgtatactttgaaaaagataaacagatgttccacatcattactggccccaatatgggaggtaaatcaacatatattcgacaaactggggtgatagtactcatggcccaaattgggtgttttgtgccatgtgagtcagcagaagtgtccattgtggactgcatcttagcccgagtaggggctggtgacagtcaattgaaaggagtctccacgttcatggctgaaatgttggaaactgcttctatcctcaggtctgcaaccaaagattcattaataatcatagatgaattgggaagaggaacttctacctacgatggatttgggttagcatgggctatatcagaatacattgcaacaaagattggtgctttttgcatgtttgcaacccattttcatgaacttactgccttggccaatcagataccaactgttaataatctacatgtcacagcactcaccactgaagagaccttaactatgctttatcaggtgaagaaaggtgtctgtgatcaaagttttgggattcatgttgcagagcttgctaatttccctaagcatgtaatagagtgtgctaaacagaaagccctggaacttgaggagtttcagtatattggagaatcgcaaggatatgatatcatggaaccagcagcaaagaagtgctatctggaaagagagcaaggtgaaaaaattattcaggagttcctgtccaaggtgaaacaaatgccctttactgaaatgtcagaagaaaacatcacaataaagttaaaacagctaaaagctgaagtaatagcaaagaataatagctttgtaaatgaaatcatttcacgaataaaagttactacgtga

SEQ ID NO: 26 - DNA, 4244 bp, Homo sapiens
gccgcgcggtagatgcggtgcttttaggagctccgtccgacagaacggttgggccttgccggctgtcggtatgtcgcgacagagcaccctgtacagcttcttccccaagtctccggcgctgagtgatgccaacaaggcctcggccagggcctcacgcgaaggcggccgtgccgccgctgcccccggggcctctccttccccaggcggggatgcggcctggagcgaggctgggcctgggcccaggcccttggcgcgctccgcgtcaccgcccaaggcgaagaacctcaacggagggctgcggagatcggtagcgcctgctgcccccaccagttgtgacttctcaccaggagatttggtttgggccaagatggagggttacccctggtggccttgtctggtttacaaccacccctttgatggaacattcatccgcgagaaagggaaatcagtccgtgttcatgtacagttttttgatgacagcccaacaaggggctgggttagcaaaaggcttttaaagccatatacaggttcaaaatcaaaggaagcccagaagggaggtcatttttacagtgcaaagcctgaaatactgagagcaatgcaacgtgcagatgaagccttaaataaagacaagattaagaggcttgaattggcagtttgtgatgagccctcagagccagaagaggaagaagagatggaggtaggcacaacttacgtaacagataagagtgaagaagataatgaaattgagagtgaagaggaagtacagcctaagacacaaggatctaggcgaagtagccgccaaataaaaaaacgaagggtcatatcagattctgagagtgacattggtggctctgatgtggaatttaagccagacactaaggaggaaggaagcagtgatgaaataagcagtggagtgggggatagtgagagtgaaggcctgaacagccctgtcaaagttgctcgaaagcggaagagaatggtgactggaaatggctctcttaaaaggaaaagctctaggaaggaaacgccctcagccaccaaacaagcaactagcatttcatcagaaaccaagaatactttgagagctttctctgcccctcaaaattctgaatcccaagcccacgttagtggaggtggtgatgacagtagtcgccctactgtttggtatcatgaaactttagaatggcttaaggaggaaaagagaagagatgagcacaggaggaggcctgatcaccccgattttgatgcatctacactctatgtgcctgaggatttcctcaattcttgtactcctgggatgaggaagtggtggcagattaagtctcagaactttgatcttgtcatctgttacaaggtggggaaattttatgagctgtaccacatggatgctcttattggagtcagtgaactggggctggtattcatgaaaggcaactgggcccattctggctttcctgaaattgcatttggccgttattcagattccctggtgcagaagggctataaagtagcacgagtggaacagactgagactccagaaatgatggaggcacgatgtagaaagatggcacatatatccaagtatgatagagtggtgaggagggagatctgtaggatcattaccaagggtacacagacttacagtgtgctggaaggtgatccctctgagaactacagtaagtatcttcttagcctcaaagaaaaagaggaagattcttctggccatactcgtgcatatggtgtgtgctttgttgatacttcactgggaaagtttttcataggtcagttttcagatgatcgccattgttcgagatttaggactctagtggcacactatcccccagtacaagttttatttgaaaaaggaaatctctcaaaggaaactaaaacaattctaaagagttcattgtcctgttctcttcaggaaggtctgatacccggctcccagttttgggatgcatccaaaactttgagaactctccttgaggaagaatattttagggaaaagctaagtgatggcattggggtgatgttaccccaggtgcttaaaggtatgacttcagagtctgattccattgggttgacaccaggagagaaaagtgaattggccctctctgctctaggtggttgtgtcttctacctcaaaaaatgccttattgatcaggagcttttatcaatggctaattttgaagaatatattcccttggattctgacacagtcagcactacaagatctggtgctatcttcaccaaagcctatcaacgaatggtgctagatgcagtgacattaaacaacttggagatttttctgaatggaacaaatggttctactgaaggaaccctactagagagggttgatacttgccatactccttttggtaagcggctcctaaagcaatggctttgtgccccactctgtaaccattatgctattaatgatcgtctagatgccatagaagacctcatggttgtgcctgacaaaatctccgaagttgtagagcttctaaagaagcttccagatcttgagaggctactcagtaaaattcataatgttgggtctcccctgaagagtcagaaccacccagacagcagggctataatgtatgaagaaactacatacagcaagaagaagattattgattttctttctgctctggaaggattcaaagtaatgtgtaaaattatagggatcatggaagaagttgctgatggttttaagtctaaaatccttaagcaggtcatctctctgcagacaaaaaatcctgaaggtcgttttcctgatttgactgtagaattgaaccgatgggatacagcctttgaccatgaaaaggctcgaaagactggacttattactcccaaagcaggctttgactctgattatgaccaagctcttgctgacataagagaaaatgaacagagcctcctggaatacctagagaaacagcgcaacagaattggctgtaggaccatagtctattgggggattggtaggaaccgttaccagctggaaattcctgagaatttcaccactcgcaatttgccagaagaatacgagttgaaatctaccaagaagggctgtaaacgatactggaccaaaactattgaaaagaagttggctaatctcataaatgctgaagaacggagggatgtatcattgaaggactgcatgcggcgactgttctataactttgataaaaattacaaggactggcagtctgctgtagagtgtatcgcagtgttggatgttttactgtgcctggctaactatagtcgagggggtgatggtcctatgtgtcgcccagtaattctgttgccggaagataccccccccttcttagagcttaaaggatcacgccatccttgcattacgaagactttttttggagatgattttattcctaatgacattctaataggctgtgaggaagaggagcaggaaaatggcaaagcctattgtgtgcttgttactggaccaaatatggggggcaagtctacgcttatgagacaggctggcttattagctgtaatggcccagatgggttgttacgtccctgctgaagtgtgcaggctcacaccaattgatagagtgtttactagacttggtgcctcagacagaataatgtcaggtgaaagtacattttttgttgaattaagtgaaactgccagcatactcatgcatgcaacagcacattctctggtgcttgtggatgaattaggaagaggtactgcaacatttgatgggacggcaatagcaaatgcagttgttaaagaacttgctgagactataaaatgtcgtacattattttcaactcactaccattcattagtagaagattattctcaaaatgttgctgtgcgcctaggacatatggcatgcatggtagaaaatgaatgtgaagaccccagccaggagactattacgttcctctataaattcattaagggagcttgtcctaaaagctatggctttaatgcagcaaggcttgctaatctcccagaggaagttattcaaaagggacatagaaaagcaagagaatttgagaagatgaatcagtcactacgattatttcgggaagtttgcctggctagtgaaaggtcaactgtagatgctgaagctgtccataaattgctgactttgattaaggaattatagactgactacattggaagctttgagttgacttctgacaaaggtggtaaattcagacaacattatgatctaataaactttattttttaaaaat

SEQ ID NO: 27 - DNA, 2589 bp, Homo sapiens
atggagcgagctgagagctcgagtacagaacctgctaaggccatcaaacctattgatcggaagtcagtccatcagatttgctctgggcaggtggtactgagtctaagcactgcggtaaaggagttagtagaaaacagtctggatgctggtgccactaatattgatctaaagcttaaggactatggagtggatcttattgaagtttcagacaatggatgtggggtagaagaagaaaacttcgaaggcttaactctgaaacatcacacatctaagattcaagagtttgccgacctaactcaggttgaaacttttggctttcggggggaagctctgagctcactttgtgcactgagcgatgtcaccatttctacctgccacgcatcggcgaaggttggaactcgactgatgtttgatcacaatgggaaaattatccagaaaaccccctacccccgccccagagggaccacagtcagcgtgcagcagttattttccacactacctgtgcgccataaggaatttcaaaggaatattaagaaggagtatgccaaaatggtccaggtcttacatgcatactgtatcatttcagcaggcatccgtgtaagttgcaccaatcagcttggacaaggaaaacgacagcctgtggtatgcacaggtggaagccccagcataaaggaaaatatcggctctgtgtttgggcagaagcagttgcaaagcctcattccttttgttcagctgccccctagtgactccgtgtgtgaagagtacggtttgagctgttccgatgctctgcataatcttttttacatctcaggtttcatttcacaatgcacgcatggagttggaaggagttcaacagacagacagtttttctttatcaaccggcggccttgtgacccagcaaaggtctgcagactcgtgaatgaggtctaccacatgtataatcgacaccagtatccatttgttgttcttaacatttctgttgattcagaatgcgttgatatcaatgttactccagataaaaggcaaattttgctacaagaggaaaagcttttgttggcagttttaaagacctctttgataggaatgtttgatagtgatgtcaacaagctaaatgtcagtcagcagccactgctggatgttgaaggtaacttaataaaaatgcatgcagcggatttggaaaagcccatggtagaaaagcaggatcaatccccttcattaaggactggagaagaaaaaaaagacgtgtccatttccagactgcgagaggccttttctcttcgtcacacaacagagaacaagcctcacagcccaaagactccagaaccaagaaggagccctctaggacagaaaaggggtatgctgtcttctagcacttcaggtgccatctctgacaaaggcgtcctgagacctcagaaagaggcagtgagttccagtcacggacccagtgaccctacggacagagcggaggtggagaaggactcggggcacggcagcacttccgtggattctgaggggttcagcatcccagacacgggcagtcactgcagcagcgagtatgcggccagctccccaggggacaggggctcgcaggaacatgtggactctcaggagaaagcgcctaaaactgacgactctttttcagatgtggactgccattcaaaccaggaagataccggatgtaaatttcgagttttgcctcagccaactaatctcgcaaccccaaacacaaagcgttttaaaaaagaagaaattctttccagttctgacatttgtcaaaagttagtaaatactcaggacatgtcagcctctcaggttgatgtagctgtgaaaattaataagaaagttgtgcccctggacttttctatgagttctttagctaaacgaataaagcagttacatcatgaagcacagcaaagtgaaggggaacagaattacaggaagtttagggcaaagatttgtcctggagaaaatcaagcagccgaagatgaactaagaaaagagataagtaaaacgatgtttgcagaaatggaaatcattggtcagtttaacctgggatttataataaccaaactgaatgaggatatcttcatagtggaccagcatgccacggacgagaagtataacttcgagatgctgcagcagcacaccgtgctccaggggcagaggctcatagcacctcagactctcaacttaactgctgttaatgaagctgttctgatagaaaatctggaaatatttagaaagaatggctttgattttgttatcgatgaaaatgctccagtcactgaaagggctaaactgatttccttgccaactagtaaaaactggaccttcggaccccaggacgtcgatgaactgatcttcatgctgagcgacagccctggggtcatgtgccggccttcccgagtcaagcagatgtttgcctccagagcctgccggaagtcggtgatgattgggactgctcttaacacaagcgagatgaagaaactgatcacccacatgggggagatggaccacccctggaactgtccccatggaaggccaaccatgagacacatcgccaacctgggtgtcatttctcagaactga